SEQUENCE LISTING (231 sequences)

SEQ ID NO: 1 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEEAAXQRFLEHLV

SEQ ID NO: 2 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEFAAAQRFLEHLV

SEQ ID NO: 3 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKELAAQQRFLEHLV

SEQ ID NO: 4 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKETAATQRFLEHLV

SEQ ID NO: 5 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEXAAQQRFLEHLV

SEQ ID NO: 6 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEEAASQRFLEHLV

SEQ ID NO: 7 - protein, 27 aa, synthetic construct
  SITE 2: note = 2-aminoisobutyric acid
  SITE 16: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEXAAXQRFLEHLV

SEQ ID NO: 8 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEFAASQRFLEHLV

SEQ ID NO: 9 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKELAAAQRFLEHLV

SEQ ID NO: 10 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKELAAXQRFLEHLV

SEQ ID NO: 11 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKELAAAQRFLEHLV

SEQ ID NO: 12 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKELAASQRFLEHLV

SEQ ID NO: 13 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEXAATQRFLEHLV

SEQ ID NO: 14 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAAQQRFLEHLV

SEQ ID NO: 15 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEXAAAQRFLEHLV

SEQ ID NO: 16 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEXAAEQRFLEHLV

SEQ ID NO: 17 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEEAASQRFLEHLV

SEQ ID NO: 18 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEXAAEQRFLEHLV

SEQ ID NO: 19 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKELAASQRFLEHLV

SEQ ID NO: 20 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKETAASQRFLEHLV

SEQ ID NO: 21 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKETAAQQRFLEHLV

SEQ ID NO: 22 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEEAAEQRFLEHLV

SEQ ID NO: 23 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKETAAAQRFLEHLV

SEQ ID NO: 24 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEFAAQQRFLEHLV

SEQ ID NO: 25 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKETAAXQRFLEHLV

SEQ ID NO: 26 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKETAATQRFLEHLV

SEQ ID NO: 27 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEFAAXQRFLEHLV

SEQ ID NO: 28 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEEAAAQRFLEHLV

SEQ ID NO: 29 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEFAAAQRFLEHLV

SEQ ID NO: 30 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAATQRFLEHLV

SEQ ID NO: 31 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEFAAEQRFLEHLV

SEQ ID NO: 32 - protein, 27 aa, synthetic construct
  SITE 2: note = 2-aminoisobutyric acid
  SITE 16: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEXAAXQRFLEHLV

SEQ ID NO: 33 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKELAAQQRFLEHLV

SEQ ID NO: 34 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKETAAEQRFLEHLV

SEQ ID NO: 35 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEEAAXQRFLEHLV

SEQ ID NO: 36 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKELAAEQRFLEHLV

SEQ ID NO: 37 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEEAAXQRFLEHLV

SEQ ID NO: 38 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEEAASQRFLEHLV

SEQ ID NO: 39 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEEAAQQRFLEHLV

SEQ ID NO: 40 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEXAAQQRFLEHLV

SEQ ID NO: 41 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKETAAQQRFLEHLV

SEQ ID NO: 42 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKETAAEQRFLEHLV

SEQ ID NO: 43 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEXAAAQRFLEHLV

SEQ ID NO: 44 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKETAAAQRFLEHLV

SEQ ID NO: 45 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKETAAXQRFLEHLV

SEQ ID NO: 46 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEFAAXQRFLEHLV

SEQ ID NO: 47 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAAQQRFLEHLV

SEQ ID NO: 48 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEXAATQRFLEHLV

SEQ ID NO: 49 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEXAATQRFLEHLV

SEQ ID NO: 50 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEXAATQRFLEHLV

SEQ ID NO: 51 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAAEQRFLEHLV

SEQ ID NO: 52 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEFAASQRFLEHLV

SEQ ID NO: 53 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEEAATQRFLEHLV

SEQ ID NO: 54 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKETAATQRFLEHLV

SEQ ID NO: 55 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAATQRFLEHLV

SEQ ID NO: 56 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEEAAEQRFLEHLV

SEQ ID NO: 57 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKETAAAQRFLEHLV

SEQ ID NO: 58 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKETAAQQRFLEHLV

SEQ ID NO: 59 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEXAASQRFLEHLV

SEQ ID NO: 60 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEEAAAQRFLEHLV

SEQ ID NO: 61 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAAAQRFLEHLV

SEQ ID NO: 62 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKETAAAQRFLEHLV

SEQ ID NO: 63 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEEAAAQRFLEHLV

SEQ ID NO: 64 - protein, 27 aa, synthetic construct
  SITE 2: note = 2-aminoisobutyric acid
  SITE 16: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEXAAXQRFLEHLV

SEQ ID NO: 65 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEEAATQRFLEHLV

SEQ ID NO: 66 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEFAASQRFLEHLV

SEQ ID NO: 67 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKELAATQRFLEHLV

SEQ ID NO: 68 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEFAAXQRFLEHLV

SEQ ID NO: 69 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKETAAEQRFLEHLV

SEQ ID NO: 70 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKETAATQRFLEHLV

SEQ ID NO: 71 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKETAASQRFLEHLV

SEQ ID NO: 72 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEFAAEQRFLEHLV

SEQ ID NO: 73 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEEAAQQRFLEHLV

SEQ ID NO: 74 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEXAASQRFLEHLV

SEQ ID NO: 75 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAASQRFLEHLV

SEQ ID NO: 76 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEFAAQQRFLEHLV

SEQ ID NO: 77 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEEAAXQRFLEHLV

SEQ ID NO: 78 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKELAAEQRFLEHLV

SEQ ID NO: 79 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKELAAXQRFLEHLV

SEQ ID NO: 80 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKELAATQRFLEHLV

SEQ ID NO: 81 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKEXAAAQRFLEHLV

SEQ ID NO: 82 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEXAATQRFLEHLV

SEQ ID NO: 83 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEEAAQQRFLEHLV

SEQ ID NO: 84 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEFAAAQRFLEHLV

SEQ ID NO: 85 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSRLKETAAXQRFLEHLV

SEQ ID NO: 86 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKELAAAQRFLEHLV

SEQ ID NO: 87 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAASQRFLEHLV

SEQ ID NO: 88 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEXAAQQRFLEHLV

SEQ ID NO: 89 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEFAAEQRFLEHLV

SEQ ID NO: 90 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEEAAQQRFLEHLV

SEQ ID NO: 91 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEFAASQRFLEHLV

SEQ ID NO: 92 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEFAASQRFLEHLV

SEQ ID NO: 93 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKETAATQRFLEHLV

SEQ ID NO: 94 - protein, 27 aa, synthetic construct
  SITE 2: note = 2-aminoisobutyric acid
  SITE 16: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEXAAQQRFLEHLV

SEQ ID NO: 95 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAAEQRFLEHLV

SEQ ID NO: 96 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEEAAAQRFLEHLV

SEQ ID NO: 97 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAAXQRFLEHLV

SEQ ID NO: 98 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAAAQRFLEHLV

SEQ ID NO: 99 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKELAAQQRFLEHLV

SEQ ID NO: 100 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEFAAQQRFLEHLV

SEQ ID NO: 101 - protein, 27 aa, synthetic construct
  SITE 2: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKETAAXQRFLEHLV

SEQ ID NO: 102 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEEAATQRFLEHLV

SEQ ID NO: 103 - protein, 27 aa, synthetic construct
  SITE 2: note = 2-aminoisobutyric acid
  SITE 10: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDXSYLKEEAXLQRFLEHLV

SEQ ID NO: 104 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSRLLEGAALQRFIQWLV

SEQ ID NO: 105 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSGLLEGAALQRFIQWLV

SEQ ID NO: 106 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSYLLEGAALQRFIQWLV

SEQ ID NO: 107 - protein, 27 aa, synthetic construct
  SITE 2: note = 2-aminoisobutyric acid
  SITE 10: note = 2-aminoisobutyric acid
  SITE 12: note = 2-aminoisobutyric acid
  MOD_RES 27: note = C-terminal amidated (CONH2)
HXEGTFTSDXSXLLEGAALQRFIQWLV

SEQ ID NO: 108 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSWLLEGAALQRFIQWLV

SEQ ID NO: 109 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSELLEGAALQRFIQWLV

SEQ ID NO: 110 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSVLLEGAALQRFIQWLV

SEQ ID NO: 111 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSFLLEGAALQRFIQWLV

SEQ ID NO: 112 - protein, 27 aa, synthetic construct
  SITE 2: note = 2-aminoisobutyric acid
  MOD_RES 27: note = C-terminal amidated (CONH2)
HXEGTFTSDISYLLEGAALQRFIQWLV

SEQ ID NO: 113 - protein, 27 aa, synthetic construct
  SITE 2: note = 2-aminoisobutyric acid
  SITE 12: note = 2-aminoisobutyric acid
  MOD_RES 27: note = C-terminal amidated (CONH2)
HXEGTFTSDISXLLEGAALQRFIQWLV

SEQ ID NO: 114 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDISYLKEEAXLQRFLEHLV

SEQ ID NO: 115 - protein, 27 aa, synthetic construct
  SITE 2: note = 2-aminoisobutyric acid
  MOD_RES 27: note = C-terminal amidated (CONH2)
HXEGTFTSDISRLLEGAALQRFIQWLV

SEQ ID NO: 116 - protein, 27 aa, synthetic construct
  SITE 2: note = 2-aminoisobutyric acid
  MOD_RES 27: note = C-terminal amidated (CONH2)
HXEGTFTSDISVLLEGAALQRFIQWLV

SEQ ID NO: 117 - protein, 27 aa, synthetic construct
  SITE 2: note = 2-aminoisobutyric acid
  MOD_RES 27: note = C-terminal amidated (CONH2)
HXEGTFTSDISFLLEGAALQRFIQWLV

SEQ ID NO: 118 - protein, 27 aa, synthetic construct
  SITE 2: note = 2-aminoisobutyric acid
  MOD_RES 27: note = C-terminal amidated (CONH2)
HXEGTFTSDISWLLEGAALQRFIQWLV

SEQ ID NO: 119 - protein, 27 aa, synthetic construct
  SITE 2: note = 2-aminoisobutyric acid
  MOD_RES 27: note = C-terminal amidated (CONH2)
HXEGTFTSDISELLEGAALQRFIQWLV

SEQ ID NO: 120 - protein, 27 aa, synthetic construct
  SITE 2: note = 2-aminoisobutyric acid
  MOD_RES 27: note = C-terminal amidated (CONH2)
HXEGTFTSDISGLLEGAALQRFIQWLV

SEQ ID NO: 121 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDPSYLKEEAXLQRFLEHLV

SEQ ID NO: 122 - protein, 27 aa, synthetic construct
  SITE 2: note = 2-aminoisobutyric acid
  MOD_RES 27: note = C-terminal amidated (CONH2)
HXEGTFTSDPSELLEGAALQRFIQWLV

SEQ ID NO: 123 - protein, 27 aa, synthetic construct
  SITE 2: note = 2-aminoisobutyric acid
  MOD_RES 27: note = C-terminal amidated (CONH2)
HXEGTFTSDPSGLLEGAALQRFIQWLV

SEQ ID NO: 124 - protein, 27 aa, synthetic construct
  SITE 2: note = 2-aminoisobutyric acid
  MOD_RES 27: note = C-terminal amidated (CONH2)
HXEGTFTSDPSRLLEGAALQRFIQWLV

SEQ ID NO: 125 - protein, 27 aa, synthetic construct
  SITE 2: note = 2-aminoisobutyric acid
  SITE 12: note = 2-aminoisobutyric acid
  MOD_RES 27: note = C-terminal amidated (CONH2)
HXEGTFTSDPSXLLEGAALQRFIQWLV

SEQ ID NO: 126 - protein, 27 aa, synthetic construct
  SITE 2: note = 2-aminoisobutyric acid
  MOD_RES 27: note = C-terminal amidated (CONH2)
HXEGTFTSDPSFLLEGAALQRFIQWLV

SEQ ID NO: 127 - protein, 27 aa, synthetic construct
  SITE 2: note = 2-aminoisobutyric acid
  MOD_RES 27: note = C-terminal amidated (CONH2)
HXEGTFTSDPSWLLEGAALQRFIQWLV

SEQ ID NO: 128 - protein, 27 aa, synthetic construct
  SITE 2: note = 2-aminoisobutyric acid
  MOD_RES 27: note = C-terminal amidated (CONH2)
HXEGTFTSDPSYLLEGAALQRFIQWLV

SEQ ID NO: 129 - protein, 27 aa, synthetic construct
  SITE 2: note = 2-aminoisobutyric acid
  MOD_RES 27: note = C-terminal amidated (CONH2)
HXEGTFTSDPSVLLEGAALQRFIQWLV

SEQ ID NO: 130 - protein, 27 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 27: note = C-terminal amidated (CONH2)
HXEGTFTSDVSYLWEEAXLQRFLEKLV

SEQ ID NO: 131 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXNQRFLEHLV

SEQ ID NO: 132 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEALLQRFLEHLV

SEQ ID NO: 133 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRFLEFLV

SEQ ID NO: 134 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXSQRFLEHLV

SEQ ID NO: 135 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRWLEHLV

SEQ ID NO: 136 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSNLKEEAXLQRFLEHLV

SEQ ID NO: 137 - protein, 27 aa, synthetic construct
  SITE 2: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAALQRFLEHLV

SEQ ID NO: 138 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSVLKEEAXLQRFLEHLV

SEQ ID NO: 139 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXIQRFLEHLV

SEQ ID NO: 140 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSPLKEEAXLQRFLEHLV

SEQ ID NO: 141 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRYLEHLV

SEQ ID NO: 142 - protein, 27 aa, synthetic construct
  SITE 18: note = 2-aminoisobutyric acid
  LIPID 14: note = C20DA[gamma-Glu][OEG][OEG]-
  MOD_RES 27: note = C-terminal amidated (CONH2)
HGEGTFTSDVSYLKEEAXLQRFLEHLV

SEQ ID NO: 143 - protein, 27 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 27: note = C-terminal amidated (CONH2)
HXEGTFTSDVSYLYEEAXLQRFLEKLV

SEQ ID NO: 144 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRFLEYLV

SEQ ID NO: 145 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSHLKEEAXLQRFLEHLV

SEQ ID NO: 146 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXAQRFLEHLV

SEQ ID NO: 147 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRFLEKLV

SEQ ID NO: 148 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSALKEEAXLQRFLEHLV

SEQ ID NO: 149 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSLLKEEAXLQRFLEHLV

SEQ ID NO: 150 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXEQRFLEHLV

SEQ ID NO: 151 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRFLEELV

SEQ ID NO: 152 - protein, 27 aa, synthetic construct
  SITE 2: note = 2-aminoisobutyric acid
  SITE 18: note = 2-aminoisobutyric acid
  SITE 19: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXXQRFLEHLV

SEQ ID NO: 153 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSDLKEEAXLQRFLEHLV

SEQ ID NO: 154 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXGQRFLEHLV

SEQ ID NO: 155 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXVGTFTSDVSYLKEEAXLQRFLEHLV

SEQ ID NO: 156 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXQGTFTSDVSYLKEEAXLQRFLEHLV

SEQ ID NO: 157 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXQQRFLEHLV

SEQ ID NO: 158 - protein, 27 aa, synthetic construct
  SITE 2: note = 2-aminoisobutyric acid
  SITE 14: note = 2-aminoisobutyric acid
  SITE 18: note = 2-aminoisobutyric acid
  LIPID 25: note = C20DA[gamma-Glu][OEG][OEG]-
  MOD_RES 27: note = C-terminal amidated (CONH2)
HXEGTFTSDVSYLXEEAXLQRFLEKLV

SEQ ID NO: 159 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXYGTFTSDVSYLKEEAXLQRFLEHLV

SEQ ID NO: 160 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXWGTFTSDVSYLKEEAXLQRFLEHLV

SEQ ID NO: 161 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXVQRFLEHLV

SEQ ID NO: 162 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXTQRFLEHLV

SEQ ID NO: 163 - protein, 27 aa, synthetic construct
  SITE 2: note = 2-aminoisobutyric acid
  SITE 12: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSXLKEEAXLQRFLEHLV

SEQ ID NO: 164 - protein, 27 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 27: note = C-terminal amidated (CONH2)
HXEGTFTSDVSYLHEEAXLQRFLEKLV

SEQ ID NO: 165 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSTLKEEAXLQRFLEHLV

SEQ ID NO: 166 - protein, 27 aa, synthetic construct
  SITE 18: note = 2-aminoisobutyric acid
  LIPID 14: note = C20DA[gamma-Glu][OEG][OEG]-
  MOD_RES 27: note = C-terminal amidated (CONH2)
HAEGTFTSDVSYLKEEAXLQRFLEHLV

SEQ ID NO: 167 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSGLKEEAXLQRFLEHLV

SEQ ID NO: 168 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSELKEEAXLQRFLEHLV

SEQ ID NO: 169 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRFLEWLV

SEQ ID NO: 170 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXLGTFTSDVSYLKEEAXLQRFLEHLV

SEQ ID NO: 171 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXHGTFTSDVSYLKEEAXLQRFLEHLV

SEQ ID NO: 172 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSILKEEAXLQRFLEHLV

SEQ ID NO: 173 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSFLKEEAXLQRFLEHLV

SEQ ID NO: 174 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSWLKEEAXLQRFLEHLV

SEQ ID NO: 175 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXIGTFTSDVSYLKEEAXLQRFLEHLV

SEQ ID NO: 176 - protein, 27 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 27: note = C-terminal amidated (CONH2)
HXEGTFTSDVSYLFEEAXLQRFLEKLV

SEQ ID NO: 177 - protein, 27 aa, synthetic construct
  SITE 2: note = 2-aminoisobutyric acid
  MOD_RES 27: note = C-terminal amidated (CONH2)
HXEGTFTSDVSFLLEGAALQRFIQWLV

SEQ ID NO: 178 - protein, 27 aa, synthetic construct
  SITE 2: note = 2-aminoisobutyric acid
  MOD_RES 27: note = C-terminal amidated (CONH2)
HXEGTFTSDVSRLLEGAALQRFIQWLV

SEQ ID NO: 179 - protein, 27 aa, synthetic construct
  SITE 2: note = 2-aminoisobutyric acid
  SITE 12: note = 2-aminoisobutyric acid
  MOD_RES 27: note = C-terminal amidated (CONH2)
HXEGTFTSDVSXLLEGAALQRFIQWLV

SEQ ID NO: 180 - protein, 27 aa, synthetic construct
  SITE 2: note = 2-aminoisobutyric acid
  MOD_RES 27: note = C-terminal amidated (CONH2)
HXEGTFTSDVSELLEGAALQRFIQWLV

SEQ ID NO: 181 - protein, 27 aa, synthetic construct
  SITE 2: note = 2-aminoisobutyric acid
  MOD_RES 27: note = C-terminal amidated (CONH2)
HXEGTFTSDVSYLLEGAALQRFIQWLV

SEQ ID NO: 182 - protein, 27 aa, synthetic construct
  SITE 2: note = 2-aminoisobutyric acid
  MOD_RES 27: note = C-terminal amidated (CONH2)
HXEGTFTSDVSGLLEGAALQRFIQWLV

SEQ ID NO: 183 - protein, 27 aa, synthetic construct
  SITE 2: note = 2-aminoisobutyric acid
  MOD_RES 27: note = C-terminal amidated (CONH2)
HXEGTFTSDVSWLLEGAALQRFIQWLV

SEQ ID NO: 184 - protein, 27 aa, synthetic construct
  SITE 2: note = 2-aminoisobutyric acid
  MOD_RES 27: note = C-terminal amidated (CONH2)
HXEGTFTSDVSVLLEGAALQRFIQWLV

SEQ ID NO: 185 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDWSYLKEEAXLQRFLEHLV

SEQ ID NO: 186 - protein, 27 aa, synthetic construct
  SITE 2: note = 2-aminoisobutyric acid
  MOD_RES 27: note = C-terminal amidated (CONH2)
HXEGTFTSDWSRLLEGAALQRFIQWLV

SEQ ID NO: 187 - protein, 27 aa, synthetic construct
  SITE 2: note = 2-aminoisobutyric acid
  MOD_RES 27: note = C-terminal amidated (CONH2)
HXEGTFTSDWSGLLEGAALQRFIQWLV

SEQ ID NO: 188 - protein, 27 aa, synthetic construct
  SITE 2: note = 2-aminoisobutyric acid
  MOD_RES 27: note = C-terminal amidated (CONH2)
HXEGTFTSDWSVLLEGAALQRFIQWLV

SEQ ID NO: 189 - protein, 27 aa, synthetic construct
  SITE 2: note = 2-aminoisobutyric acid
  MOD_RES 27: note = C-terminal amidated (CONH2)
HXEGTFTSDWSYLLEGAALQRFIQWLV

SEQ ID NO: 190 - protein, 27 aa, synthetic construct
  SITE 2: note = 2-aminoisobutyric acid
  MOD_RES 27: note = C-terminal amidated (CONH2)
HXEGTFTSDWSFLLEGAALQRFIQWLV

SEQ ID NO: 191 - protein, 27 aa, synthetic construct
  SITE 2: note = 2-aminoisobutyric acid
  MOD_RES 27: note = C-terminal amidated (CONH2)
HXEGTFTSDWSWLLEGAALQRFIQWLV

SEQ ID NO: 192 - protein, 27 aa, synthetic construct
  SITE 2: note = 2-aminoisobutyric acid
  SITE 12: note = 2-aminoisobutyric acid
  MOD_RES 27: note = C-terminal amidated (CONH2)
HXEGTFTSDWSXLLEGAALQRFIQWLV

SEQ ID NO: 193 - protein, 27 aa, synthetic construct
  SITE 2: note = 2-aminoisobutyric acid
  MOD_RES 27: note = C-terminal amidated (CONH2)
HXEGTFTSDWSELLEGAALQRFIQWLV

SEQ ID NO: 194 - protein, 27 aa, synthetic construct
  SITE 2: note = 2-aminoisobutyric acid
  MOD_RES 27: note = C-terminal amidated (CONH2)
HXEGTFTSDYSRLLEGAALQRFIQWLV

SEQ ID NO: 195 - protein, 27 aa, synthetic construct
  SITE 2: note = 2-aminoisobutyric acid
  MOD_RES 27: note = C-terminal amidated (CONH2)
HXEGTFTSDYSGLLEGAALQRFIQWLV

SEQ ID NO: 196 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDYSYLKEEAXLQRFLEHLV

SEQ ID NO: 197 - protein, 27 aa, synthetic construct
  SITE 2: note = 2-aminoisobutyric acid
  SITE 12: note = 2-aminoisobutyric acid
  MOD_RES 27: note = C-terminal amidated (CONH2)
HXEGTFTSDYSXLLEGAALQRFIQWLV

SEQ ID NO: 198 - protein, 27 aa, synthetic construct
  SITE 2: note = 2-aminoisobutyric acid
  MOD_RES 27: note = C-terminal amidated (CONH2)
HXEGTFTSDYSVLLEGAALQRFIQWLV

SEQ ID NO: 199 - protein, 27 aa, synthetic construct
  SITE 2: note = 2-aminoisobutyric acid
  MOD_RES 27: note = C-terminal amidated (CONH2)
HXEGTFTSDYSELLEGAALQRFIQWLV

SEQ ID NO: 200 - protein, 27 aa, synthetic construct
  SITE 2: note = 2-aminoisobutyric acid
  MOD_RES 27: note = C-terminal amidated (CONH2)
HXEGTFTSDYSWLLEGAALQRFIQWLV

SEQ ID NO: 201 - protein, 27 aa, synthetic construct
  SITE 2: note = 2-aminoisobutyric acid
  MOD_RES 27: note = C-terminal amidated (CONH2)
HXEGTFTSDYSYLLEGAALQRFIQWLV

SEQ ID NO: 202 - protein, 27 aa, synthetic construct
  SITE 2: note = 2-aminoisobutyric acid
  MOD_RES 27: note = C-terminal amidated (CONH2)
HXEGTFTSDYSFLLEGAALQRFIQWLV

SEQ ID NO: 203 - protein, 27 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 27: note = C-terminal amidated (CONH2)
HXEGTFTSDVSYLKEEAXLQRFLEHLV

SEQ ID NO: 204 - protein, 30 aa, synthetic construct
  SITE 2: note = 2-aminoisobutyric acid
  LIPID 14: note = C18DA[gamma-Glu][OEG][OEG]-
  MOD_RES 30: note = C-terminal amidated (CONH2)
HXEGTFTSDVSRLKEGAAEQRFLQHLVEAE

SEQ ID NO: 205 - protein, 30 aa, synthetic construct
  SITE 2: note = 2-aminoisobutyric acid
  LIPID 14: note = C18DA[gamma-Glu][OEG][OEG]-
  MOD_RES 30: note = C-terminal amidated (CONH2)
HXEGTFTSDVSRLKEGAAEQRFLQHLVEAA

SEQ ID NO: 206 - protein, 30 aa, synthetic construct
  SITE 2: note = 2-aminoisobutyric acid
  LIPID 14: note = C18DA[gamma-Glu][OEG][OEG]-
  MOD_RES 30: note = C-terminal amidated (CONH2)
HXEGTFTSDVSRLKEGAAEQRFLQHLVAAE

SEQ ID NO: 207 - protein, 30 aa, synthetic construct
  SITE 2: note = 2-aminoisobutyric acid
  LIPID 14: note = C20DA[gamma-Glu][OEG][OEG]-
  MOD_RES 30: note = C-terminal amidated (CONH2)
HXEGTFTSDVSRLKEEAALQRFLEHLVEAA

SEQ ID NO: 208 - protein, 30 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 30: note = C-terminal amidated (CONH2)
HXEGTFTSDVSRLKEEAXLQRFLEHLVEAA

SEQ ID NO: 209 - protein, 30 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 30: note = C-terminal amidated (CONH2)
HXEGTFTSDVSYLKEEAXLQRFLEHLVEAA

SEQ ID NO: 210 - protein, 30 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 30: note = C-terminal amidated (CONH2)
HXEGTFTSDVSELKEEAXLQRFLEHLVEAA

SEQ ID NO: 211 - protein, 30 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 30: note = C-terminal amidated (CONH2)
HXEGTFTSDVSTLKEEAXLQRFLEHLVEAA

SEQ ID NO: 212 - protein, 30 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 30: note = C-terminal amidated (CONH2)
HXEGTFTSDVSRLLEEAXLQRFLEKLVEAA

SEQ ID NO: 213 - protein, 30 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 30: note = C-terminal amidated (CONH2)
HXEGTFTSDVSYLLEEAXLQRFLEKLVEAA

SEQ ID NO: 214 - protein, 30 aa, synthetic construct
  SITE 2: note = 2-aminoisobutyric acid
  SITE 18: note = 2-aminoisobutyric acid
  LIPID 25: note = C20DA[gamma-Glu][OEG][OEG]-
  MOD_RES 30: note = C-terminal amidated (CONH2)
HXEGTFTSDVSELLEEAXLQRFLEKLVEAA

SEQ ID NO: 215 - protein, 30 aa, synthetic construct
  SITE 2: note = 2-aminoisobutyric acid
  SITE 18: note = 2-aminoisobutyric acid
  LIPID 14: note = C20DA[gamma-Glu][OEG][OEG]-
  MOD_RES 30: note = C-terminal amidated (CONH2)
HXEGTFTSDVSRLKEEAXAQRFLEHLVEAA

SEQ ID NO: 216 - protein, 27 aa, synthetic construct
  SITE 2: note = 2-aminoisobutyric acid
  MOD_RES 27: note = C-terminal amidated (CONH2)
HXEGTFTSDLSRLLEGAALQRFIQWLV

SEQ ID NO: 217 - protein, 27 aa, synthetic construct
  SITE 2: note = 2-aminoisobutyric acid
  MOD_RES 27: note = C-terminal amidated (CONH2)
HXEGTFTSDYSRLLEGAALQRFIQWLV

SEQ ID NO: 218 - protein, 27 aa, synthetic construct
  SITE 2: note = 2-aminoisobutyric acid
  MOD_RES 27: note = C-terminal amidated (CONH2)
HXEGTFTSDISRLLEGAALQRFIQWLV

SEQ ID NO: 219 - protein, 27 aa, synthetic construct
  SITE 2: note = 2-aminoisobutyric acid
  MOD_RES 27: note = C-terminal amidated (CONH2)
HXEGTFTSDWSRLLEGAALQRFIQWLV

SEQ ID NO: 220 - protein, 27 aa, synthetic construct
  SITE 2: note = 2-aminoisobutyric acid
  MOD_RES 27: note = C-terminal amidated (CONH2)
HXEGTFTSDVSRLLEGAALQRFIQWLV

SEQ ID NO: 221 - protein, 27 aa, synthetic construct
  SITE 2: note = 2-aminoisobutyric acid
  MOD_RES 27: note = C-terminal amidated (CONH2)
HXEGTFTSDPSRLLEGAALQRFIQWLV

SEQ ID NO: 222 - protein, 27 aa, synthetic construct
  SITE 2: note = 2-aminoisobutyric acid
  SITE 10: note = 2-aminoisobutyric acid
  MOD_RES 27: note = C-terminal amidated (CONH2)
HXEGTFTSDXSRLLEGAALQRFIQWLV

SEQ ID NO: 223 - protein, 27 aa, synthetic construct
  SITE 2: note = 2-aminoisobutyric acid
  SITE 12: note = 2-aminoisobutyric acid
  MOD_RES 27: note = C-terminal amidated (CONH2)
HXEGTFTSDLSXLLEGAALQRFIQWLV

SEQ ID NO: 224 - protein, 27 aa, synthetic construct
  SITE 2: note = 2-aminoisobutyric acid
  MOD_RES 27: note = C-terminal amidated (CONH2)
HXEGTFTSDLSELLEGAALQRFIQWLV

SEQ ID NO: 225 - protein, 27 aa, synthetic construct
  SITE 2: note = 2-aminoisobutyric acid
  MOD_RES 27: note = C-terminal amidated (CONH2)
HXEGTFTSDLSFLLEGAALQRFIQWLV

SEQ ID NO: 226 - protein, 27 aa, synthetic construct
  SITE 2: note = 2-aminoisobutyric acid
  MOD_RES 27: note = C-terminal amidated (CONH2)
HXEGTFTSDLSGLLEGAALQRFIQWLV

SEQ ID NO: 227 - protein, 27 aa, synthetic construct
  SITE 2: note = 2-aminoisobutyric acid
  MOD_RES 27: note = C-terminal amidated (CONH2)
HXEGTFTSDLSVLLEGAALQRFIQWLV

SEQ ID NO: 228 - protein, 27 aa, synthetic construct
  SITE 2: note = 2-aminoisobutyric acid
  MOD_RES 27: note = C-terminal amidated (CONH2)
HXEGTFTSDLSWLLEGAALQRFIQWLV

SEQ ID NO: 229 - protein, 27 aa, synthetic construct
  SITE 2: note = 2-aminoisobutyric acid
  MOD_RES 27: note = C-terminal amidated (CONH2)
HXEGTFTSDLSYLLEGAALQRFIQWLV

SEQ ID NO: 230 - protein, 27 aa, synthetic construct
  MOD_RES 27: note = C-terminal amidated (CONH2)
HSDGTFTSELSRLREGARLQRLLQGLV

SEQ ID NO: 231 - protein, 30 aa, synthetic construct
  MOD_RES 30: note = C-terminal amidated (CONH2)
HAEGTFTSDVSSYLEGQAAKEFIAWLVKGR

(19) United States
(12) Patent Application Publication
(10) Pub. No.: US 2026/0201008 A1
(43) Pub. Date: Jul. 16, 2026

(54) SECRETIN-DERIVED GLP1R SELECTIVE AGONISTS

(71) Applicant: GUBRA A/S, Hørsholm (DK)

(72) Inventors: Jens Christian NIELSEN, Hørsholm (DK); Claudia HJØRRINGGAARD, Hørsholm (DK); Mads Mørup NYGAARD, Hørsholm (DK); Anita WESTER, Hørsholm (DK); Lisbeth ELSTER, Hørsholm (DK); Kristoffer RIGBOLT, Hørsholm (DK); Louise S. DALBØGE, Hørsholm (DK)

(73) Assignee: GUBRA A/S, Hørsholm (DK)

(21) Appl. No.: 19/487,383

(22) PCT Filed: May 24, 2024
(86) PCT No.: PCT/EP2024/064409

§ 371 Date: Nov. 24, 2025

(30) Foreign Application Priority Data
May 26, 2023 (EP) 23175692.5

(51) Int. Cl. C07K 14/605; A61K 38/00

(52) U.S. Cl. CPC C07K 14/605; A61K 38/00

(57) ABSTRACT

The present invention relates to selective GLP1R agonists. In particular, the present invention relates to the finding that the secretin (SCT) may be converted into highly potent and selective GLP1R agonists.

### FIELD OF THE INVENTION

The present invention relates to selective GLP1R agonists. In particular, the present invention relates to the finding that the secretin (SCT) may be converted into highly potent and selective GLP1R agonists. The present invention further relates to the GLP1R agonists for use as a medicament, in particular for the treatment of obesity and/or diabetes.

### BACKGROUND OF THE INVENTION

Obesity is a medical condition in which excess body fat has accumulated to the extent that it has a negative impact on health. It is affecting a huge number of individuals worldwide and increasing rapidly in certain parts of the world. The World Health Organisation (WHO) estimated that in 2016, approximately 650 million people were obese worldwide. Obesity is defined as a body mass index (BMI) above 30. Obesity is considered a major risk factor for developing a variety of medical conditions, such as cardiovascular diseases (e.g. hypertension, atherosclerosis, heart attacks, or stroke), NASH musculoskeletal disorders, certain kinds of cancers, depression, and diabetes type II, and hence is detrimental to human health.

Diabetes is a group of metabolic disorders characterized by a high blood sugar level. As of 2019, the International Diabetes Federation estimated that 463 million people are suffering from diabetes worldwide, approximately half of the individuals being diagnosed. Diabetes is divided into two types, namely type I and type II diabetes. Type I diabetes results from the pancreas's failure to produce enough insulin due to loss of beta cells caused by an autoimmune response. On the other hand, type II diabetes is a condition that begins with insulin resistance in which cells fail to respond to insulin properly and as the disease progresses may also result in a lack of insulin.

The secretin family of hormones is a group of short brain-gut peptides named after the first hormone discovered among them. Members of this family include the peptides secretin (SCT), pituitary adenylate cyclase-activating peptide (PACAP), vasoactive intestinal peptide (VIP), and growth hormone-releasing hormone (GHRH), glucagon (GCG), Glucagon-like peptide-1 (GLP-1), Glucagon-like peptide-2 (GLP-2), Glucose-dependent insulinotropic polypeptide (GIP), corticotrophin-releasing hormone (CRH), and calcitonin (CALC). These peptide hormones exert their effects through activation of the family B or family 2 of G-protein coupled receptors.

SCT is a hormone that is produced in the enteroendocrine S cells located in the intestinal glands of the duodenum and jejunum. Secretin (SCT) regulates water homeostasis throughout the body and influences the environment of the duodenum by regulating secretions in the stomach, pancreas, and liver. SCT inhibits gastric acid secretion and gastric emptying, as well as stimulating water and bicarbonate secretion from the exocrine pancreas in response to food ingestion, thereby ensuring protection of the intestinal mucosa and an optimal chemical milieu for digestive enzyme. Secretin is initially synthesized as a 120 amino acid precursor protein known as prosecretin, which contains an N-terminal signal peptide, spacer, secretin itself (residues 28-54), and a 72-amino acid C-terminal peptide. The mature secretin peptide is a linear peptide hormone, which is composed of 27 amino acids with the sequence HSDGTFTSELSRLREGARLQRLLQGLV.

Glucagon-like peptide-1 (GLP-1) is produced in enteroendocrine L cells in the intestine and secreted into the hepatic portal system in response to food intake. GLP-1 possesses several physiological properties making it, and its functional analogues, a subject of intensive investigation for the treatment of diabetes and obesity. In the stomach, GLP-1 inhibits gastric emptying, acid secretion and motility, which collectively decrease appetite. Likewise, GLP1R are highly abundant in circumventricular organs (CVOs) as well as hypothalamic nuclei involved in the regulation of energy balance and activation of GLP1R in these areas have shown to decrease food intake. Furthermore, GLP-1 functions as an incretin hormone, stimulating insulin release and inhibiting glucagon secretion from the pancreas in response to food ingestion. These actions have been clinically exploited by the development of GLP1R agonists such as Liraglutide, for use in the treatment of diabetes and obesity.

The evolutionarily related peptides of the secretin family share some sequence identity. For example, secretin and GLP-1 have 11 amino acids in common as illustrated below:

Secretin:
HSDGTFTSELSRLREGARLQRLLQGLV
GLP-1:
HAEGTFTSDVSSYLEGQAAKEFIAWLVKGR

Based on their sequence identity secretin has previously served as template to design the potent and long-acting SCTR/GLP1R co-agonist (GUB06-046) by incorporating GLP-1 residues (Witteloostuijn, 2017). The SCTR/GLP1R co-agonist GUB06-046 (SEQ ID NO: 216) has the amino acid sequence H-Aib-E-G-T-F-T-S-D-L-S-R-L-L-E-G-A-A-L-Q-R-F-I-Q-W-L-V with an SCTR EC₅₀ of 1.3 nM and an GLP1R EC₅₀ of 0.12 nM.

Despite the high number of GLP-1 analogous, Liraglutide and Semaglutide remain the only GLP-1 analogues approved for the treatment of obesity. Thus, there is a need in the art for new GLP1R agonists for use in the treatment of obesity and/or diabetes, in particular GLP1R agonists based on different chemical scaffolds. The present invention sets out to solve the problem of providing potent and selective GLP1R agonists based on the secretin scaffold.

### SUMMARY OF THE INVENTION

The present invention relates to novel GLP1R agonists based on the SCT scaffold. The present inventors found that SCT could be converted into highly potent and selective GLP1R agonists. The invention is set forth in the claims.

### BRIEF DESCRIPTION OF THE FIGURES

Embodiments of the invention will be described in more detail in the following with regard to the accompanying figures.

FIG. 1A shows the effect on hGLP1R potency by introducing the GLP-1 amino acid residue present in position X², X³, X⁹, X¹⁰, X¹², X¹³, X¹⁴, X¹⁷, X¹⁸, X¹⁹, X²⁰, X²¹, X²², X²³, X²⁴, or X²⁵ into the corresponding position of the SCT backbone. A positive mean SHAP value indicates an improved hGLP1R potency, and a negative SHAP value indicates a decreased hGLP1R potency.

FIG. 1B shows the effect on hSCTR potency by introducing the GLP-1 amino acid residue present in position X², X³, X⁹, X¹⁰, X¹², X¹³, X¹⁴, X¹⁷, X¹⁸, X¹⁹, X²⁰, X²¹, X²², X²³, X²⁴, or X²⁵ into the corresponding position of the SCT backbone. A positive mean SHAP value indicates an improved hSCTR potency, and a negative SHAP value indicates a decreased hSCTR potency.

FIG. 2 shows the normalized pEC50 values on hSCTR and hGLP1R, based on the library constructed in example 2, for the different amino acids tested in position X².

FIG. 3A shows the normalized pEC50 values on hSCTR and hGLP1R, based on the library constructed in example 2, for the different amino acids tested in position X³.

FIG. 3B shows the normalized pEC50 ratio of hSCTR and hGLP1R (i.e. selectivity), based on the library constructed in example 2, for the different amino acids tested in position X³.

FIG. 4A shows the normalized pEC50 values on hSCTR and hGLP1R, based on the library constructed in example 2, for the different amino acids tested in position X⁹.

FIG. 4B shows the normalized pEC50 ratio of hSCTR and hGLP1R (i.e. selectivity), based on the library constructed in example 2, for the different amino acids tested in position X⁹.

FIG. 5A shows the normalized pEC50 values on hSCTR and hGLP1R, based on the library constructed in example 2, for the different amino acids tested in position X¹⁰.

FIG. 5B shows the normalized pEC50 ratio of hSCTR and hGLP1R (i.e. selectivity), based on the library constructed in example 2, for the different amino acids tested in position X¹⁰.

FIG. 6A shows the normalized pEC50 values on hSCTR and hGLP1R, based on the library constructed in example 2, for the different amino acids tested in position X¹⁴.

FIG. 6B shows the normalized pEC50 ratio of hSCTR and hGLP1R (i.e. selectivity), based on the library constructed in example 2, for the different amino acids tested in position X¹⁴.

FIG. 7 shows the normalized pEC50 values on hSCTR and hGLP1R, based on the library constructed in example 2, for the different amino acids tested in position X¹⁸.

FIG. 8A shows the normalized pEC50 values on hSCTR and hGLP1R, based on the library constructed in example 2, for the different amino acids tested in position X¹⁹.

FIG. 8B shows the normalized pEC50 ratio of hSCTR and hGLP1R (i.e. selectivity), based on the library constructed in example 2, for the different amino acids tested in position X¹⁹.

FIG. 9 shows the normalized pEC50 values on hSCTR and hGLP1R, based on the library constructed in example 2, for the different amino acids tested in position X²².

FIG. 10A shows the normalized pEC50 values on hSCTR and hGLP1R, based on the library constructed in example 2, for the different amino acids tested in position X¹².

FIG. 10B shows the normalized pEC50 ratio of hSCTR and hGLP1R (i.e. selectivity), based on the library constructed in example 2, for the different amino acids tested in position X¹².

FIG. 11A shows the normalized pEC50 values on hSCTR and hGLP1R, based on the library constructed in example 2, for the different amino acids tested in position X²⁵.

FIG. 11B shows the normalized pEC50 ratio of hSCTR and hGLP1R (i.e. selectivity), based on the library constructed in example 2, for the different amino acids tested in position X²⁵.

FIG. 12 shows the effect on hGLP1R pEC50 by introducing different lipidated lysine (K) residues in position X¹-X³⁰.

FIG. 13A shows the effect on hGLP1R potency by introducing glutamate (E) residues in the indicated positions. A positive SHAP value indicates an improved hGLP1R potency, and a negative SHAP value indicates a decreased hGLP1R potency.

FIG. 14A shows the effect on fibrillation at pH 7, by introducing the indicated amino acids in the positions X³, X¹⁰, X¹², X¹⁸, X¹⁹ and X²¹. A negative SHAP value indicates that the amino acid minimizes fibrillation, whereas a positive SHAP value indicates that the amino acid increases fibrillation.

FIG. 14B shows the effect on hGLP1R potency, by introducing the indicated amino acids in the positions X³, X¹⁰, X¹², X¹⁸, X¹⁹ and X²¹. A positive SHAP value indicates an improved hGLP1R potency, and a negative SHAP value indicates a decreased hGLP1R potency.

FIG. 15A shows the effect on hGLP1R potency of extended GLP1R agonists (i.e. having from 28 to 30 amino acids). A positive mean SHAP value indicates an improved hGLP1R potency, and a negative SHAP value indicates a decreased hGLP1R potency.

FIG. 16A shows the effect on cumulative food intake of SEQ ID NO: 203 versus the vehicle.

FIG. 16B shows the % of cumulative food of SEQ ID NO: 203 intake after 24 hours versus the vehicle (vehicle set to 100%).

FIG. 17A shows the effect on cumulative food intake of SEQ ID NO: 209 versus the vehicle.

FIG. 17B shows the % of cumulative food of SEQ ID NO: 209 intake after 24 hours versus the vehicle (vehicle set to 100%).

### DEFINITIONS

The GLP1R agonists according to the present invention are preferably amidated at the C-terminal (—CONH2), like the native peptides secretin (SCT) and Glucagon-like peptide-1 (GLP-1). However, the GLP1R agonists according to the present invention may also have either a free carboxylic acid (—COOH) or another post-translational modification, such as a methyl ester (—COOMe). In the most preferred embodiment of the invention, GLP1R agonists are amidated at the C-terminal, like the peptides exemplified herein. The GLP1R agonists according to the present invention may have a free amine (—NH₂), be N-acylated (—NHCOR), N-methylated (—NHCH₃ or —N(CH₃)₂) or deaminated at the N-terminal. Most preferably, the GLP1R agonists according to the present invention have a free amine (i.e. an amino group (—NH₂)) at the N-terminal, like the peptides exemplified herein.

In the present context, lipidation refers to the covalent attachment of a lipid optionally through a linker/spacer to a GLP1R agonist according to the present invention. The lipid may be a C18DA (octadecanedioic acid), C20DA (icosanedioic acid) optionally connected through a linker/spacer consisting of one or more covalently connected units commonly used. Linkers/spacers commonly used may be but is not limited to e.g. [^{γ}E], [OEG] or [AHX] as illustrated below.

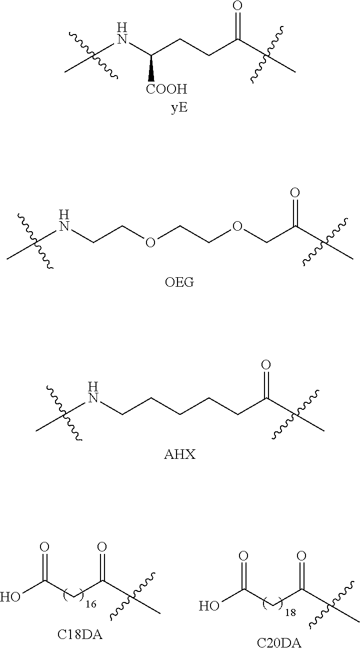

Lipidation is typically performed to improve the pharmacokinetic profile of a polypeptide by e.g. improving metabolic stability, reducing enzymatic degradation, lowering excretion and metabolism, all in all resulting in a prolonged in vivo half-life (t½). The GLP1R agonists according to the present invention may be lipidated or non-lipidated depending on the desired half-life. The polypeptides may be lipidated, e.g. at a lysine (K) residue as exemplified herein. Preferably, the lipidation is performed at a lysine residue (K) in position X¹⁴ or X²⁵. The lipid (and linker) is selected from the list consisting of C14 (tetradecanoic acid), C16 (hexadecanoic acid), C18DA, C20DA, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-. Preferably, the lipid (and linker) is selected from the list consisting of C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C20DA[^{γ}E]-, C20DA[^{γ}E][^{γ}E]-, C20DA[^{γ}E][OEG]-, C20DA[^{γ}E][OEG][OEG]-, or C20DA[^{γ}E][^{γ}E][OEG][OEG]-. Most preferably, the lipidation is C20DA[^{γ}E][OEG][OEG]-.

The amino acids herein refer to the natural amino acids (i.e. L amino acids) unless otherwise stated. The abbreviation Aib refers to 2-aminoisobutyric acid.

EC₅₀ values are used as a measure of agonist potency at the secretin receptor (SCTR) and the GLP-1 receptor (GLP1R). An EC₅₀ value is a measure of the concentration of a compound required to achieve half of that compound's maximal activity in a particular assay. pEC₅₀ is calculated as −log(EC₅₀), wherein the EC₅₀ is reported in M. If the EC₅₀ equals 1 micromolar (10⁻⁶ molar) the pEC₅₀ is 6.

The GLP1R agonists according to the present invention may be in the form of a pharmaceutically acceptable salt and/or solvate. Thus, pharmaceutically acceptable salts are intended to include, but not limited to, any salts that are commonly used in formulations of peptides. Such salts include both acid addition salts and basic salts, and examples may be found e.g. in Remington's Pharmaceutical Sciences, 17th edition.

In the present context, a selective GLP1R agonist should be understood as an agonist having a SCTR EC₅₀/GLP1R EC₅₀ ratio of at least 100, preferably a SCTR EC₅₀/GLP1R EC₅₀ ratio of at least 500, more preferably a SCTR EC₅₀/GLP1R EC₅₀ ratio of at least 1000, most preferably a SCTR EC₅₀/GLP1R EC₅₀ ratio of at least 10000, when measured using the assay and conditions as described herein. The SCTR EC₅₀/GLP1R EC₅₀ ratio is also referred to interchangeably as selectivity.

In the present context, identity to SCT is calculated by aligning the respective peptides with SCT and determining the number of amino acid residues in common. As a non-limiting example, the identity of SEQ ID NO: 1 has 16 amino acid residues in common when aligned as illustrated below thus having (16/27*100)=59% identity.

SEQ ID NO. 1:
H-X-E-G-T-F-T-S-D-V-S-R-L-K-E-E-A-A-X-Q-R-F-L-E-
H-L-V (X is Aib)
Secretin:
H-S-D-G-T-F-T-S-E-L-S-R-L-R-E-G-A-R-L-Q-R-L-L-Q-G-
L-V

As another non-limiting example, the identity of SEQ ID NO: 204 has 18 amino acid residues in common when aligned as illustrated below thus having (18/27*100)=67% identity.

SEQ ID NO. 204:
H-X-E-G-T-F-T-S-D-V-S-R-L-K-E-G-A-A-E-Q-R-F-L-Q-
H-L-V-E-A-E (X is Aib)
Secretin:
H-S-D-G-T-F-T-S-E-L-S-R-L-R-E-G-A-R-L-Q-R-L-L-Q-G-
L-V

As illustrated herein the peptides have at least 50% identity to SCT. As at least the amino acids in position X², X³, X⁹, X¹⁰, X¹⁴, X¹⁸, and X²² differs from SCT in order to provide selective GLP1R agonists, it follows that the maximum SCT identity is (27−7)/27*100=74%. The peptides exemplified herein, with the lowest SCT identity has 15 amino acids in common with SCT when aligned. Thus, in a most preferred embodiment, the peptides have at least 55% identity to SCT.

### DETAILED DESCRIPTION OF THE INVENTION

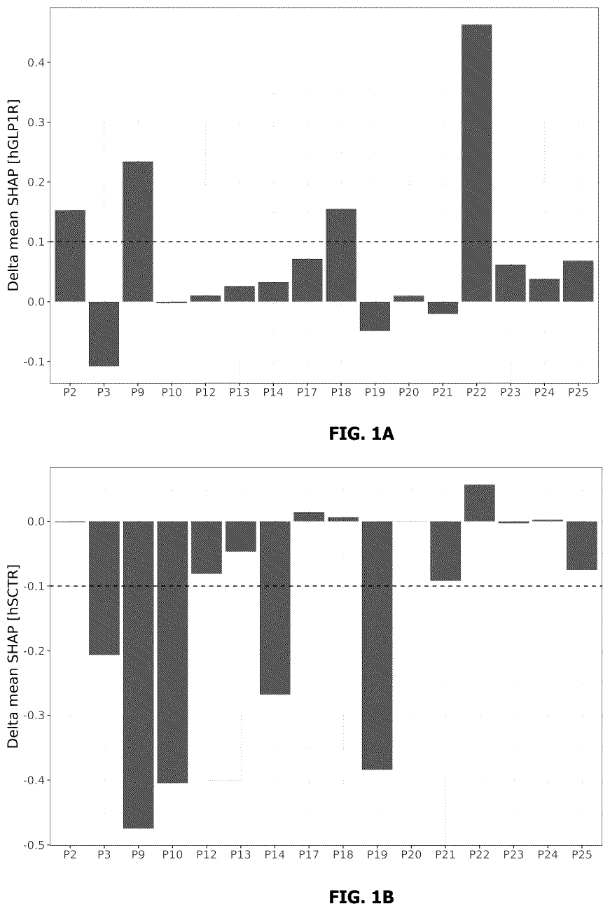

The present invention relates to novel GLP1R agonists based on the SCT scaffold. The present inventors found that SCT could be converted into potent and selective GLP1R agonists. The inventors initially constructed and tested libraries of peptides, wherein one or more amino acids in secretin was/were substituted to the corresponding amino acid found in GLP-1 (see example 1). Based on this library, the present inventors identified positions X², X⁹, X¹⁸ and X²² in SCT as being of high importance for increasing the GLP1R potency of the SCT backbone (see FIG. 1A). Likewise, the present inventors identified positions X³, X⁹, X¹⁰, X¹⁴, and X¹⁹ of SCT as being of high importance for decreasing SCTR potency of the secretin backbone to obtain selectivity. Based on these findings, the inventors further identified non-GLP-1 amino acids in position X², X³, X⁹, X¹⁰, X¹⁴, X¹⁸, X¹⁹, and X²² of SCT that were capable of converting the secretin backbone into potent and selective GLP1-R agonists (see example 2).

In summary, the inventors identified that the amino acid S in X² of SCT should be substituted with A, Aib, or G; that the amino acid D in X³ of SCT should be substituted with I, H, L, W, V, Y, E or Q; that the amino acid E in X⁹ of SCT should be substituted with D; that the amino acid L in X¹⁰ of SCT should be substituted with P, Aib, V, I, Y, or W; that the amino acid R in X¹⁴ of SCT should be substituted with F, Y, W, L, H, Aib or K; that the amino acid R in X¹⁸ of SCT should be substituted with A, Aib, or L; that the amino acid in X¹⁹ of SCT should be selected as L, N, G, S, T, Aib, A, E, Q, I or V; and that the amino acid L in X²² of SCT should be substituted with F, W or Y in order to provide selective GLP1R agonists.

The SCTR/GLP1R co-agonist GUB06-046 (SEQ ID NO: 216) having the amino acid sequence H-Aib-E-G-T-F-T-S-D-L-S-R-L-L-E-G-A-A-L-Q-R-F-I-Q-W-L-V comprises some of the identified amino acid residues capable of improving the GLP1R potency and decreasing the SCTR potency of the SCT backbone. Thus, starting from GUB06-046, the present invention relates to key finding that the GLP1R selectivity can be greatly improved by position X¹⁰. Table 3 illustrates the invention in comparison with the prior art peptide GUB06-046. As can be seen from Table 3, the substitution of the amino acid L in X¹⁰ of GUB06-046 with P, Aib, V, I, Y, or W resulted in peptides having at greatly improved selectivity (i.e. selectivity ratio of least 100) for GLP1R over SCTR, while maintaining sufficient GLP1R potency.

### Essential Positions in SCT Backbone for Providing Potent and Selective GLP1R Agonists

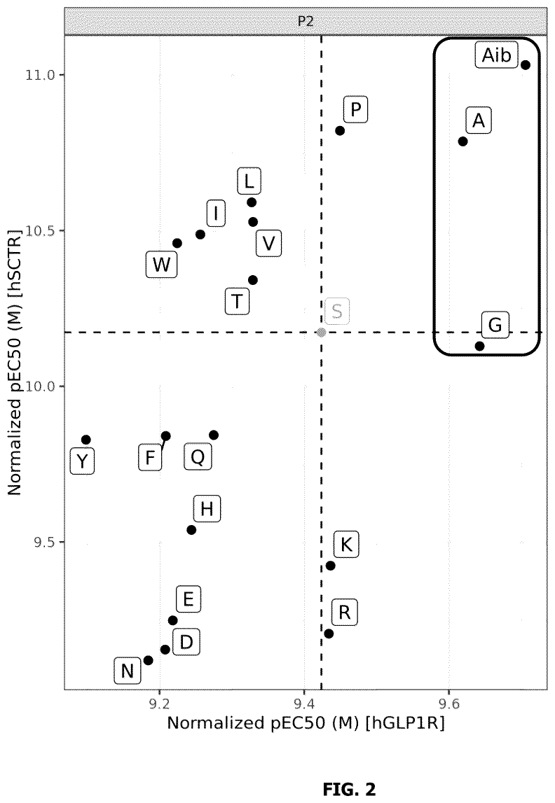

Position X²: Position X² was identified as being important for hGLP1R potency (see example 1). The deep mutational scan revealed that the amino acids Aib, A, and G resulted in the highest hGLP1R potency in position X² (see box in FIG. 2A). Thus, the inventors found that that X² should be selected as A, Aib, and G, in position X² to increase the hGLP1R potency of the SCT backbone. Most preferably, X² is selected as Aib.

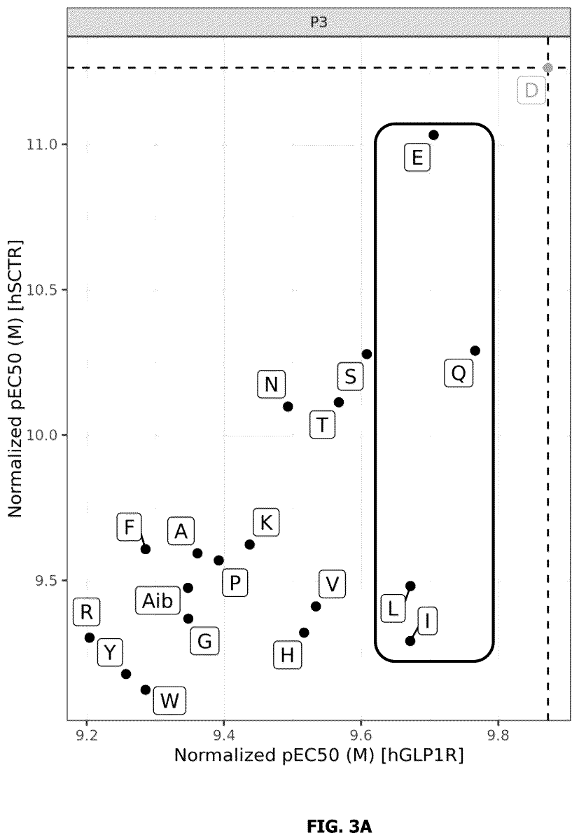

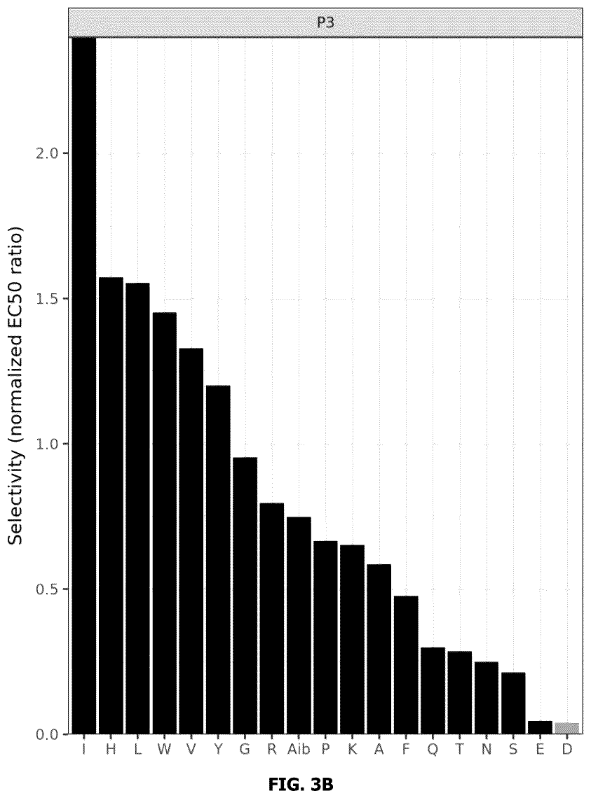

Position X³: Position X³ was identified as being important for decreasing hSCTR potency to provide selective GLP1R agonists (see example 1). The deep mutational scan revealed that in addition to the GLP-1 amino acid E (example 1), the non-GLP-1 amino acids I, H, L, W, V, Y, G, R, Aib, P, K, A, F, Q T, N, and S, were found to decrease hSCTR potency compared to the amino acid D found in SCT in position X³ (see FIG. 3A). A decrease of hSCTR potency, in position X³, should preferably be accompanied by a minimum sacrifice of hGLP1R potency to achieve the highest selectivity for GLP1R over hSCTR. In FIG. 3B, the amino acids are ranked by selectivity (i.e. hSCTR EC₅₀/hGLP1R EC₅₀). The higher the EC₅₀ ratio is for a given amino acid, the higher the selectivity for GLP1R. Thus, when position X³ is used to maximise selectivity, X³ is preferably selected from I, H, L, W, V, Y, G, R, Aib, P, K, or A (EC₅₀ ratio=0.5), more preferably X³ is selected from I, H, L, W, V, Y, G, or R (EC₅₀ ratio=0.75), even more preferably X³ is selected from I, H, L, W, V, or Y (EC₅₀ ratio=1.0), yet more preferably X³ is selected from I, H, or L (EC₅₀ ratio=1.5), most preferably X³ is selected as I. As several positions are used to decrease hSCTR potency, it is possible to include amino acids in position X³ that prioritize high hGLP1R potency, even if less optimal from a selectivity point of view. Thus, as can be seen from FIG. 3A, position X³ may be selected as E, Q, L and I to maintain maximum hGLP1R potency with varying reduction in hSCTR potency (see box in FIG. 3A). Thus, in a preferred embodiment, position X³ is selected as I, H, L, W, V, Y, E or Q to increase the selectivity. In the more preferred embodiment, X³ is selected as I, H, L, E, or Q to increase the selectivity. In the most preferred embodiment, X³ is selected as E.

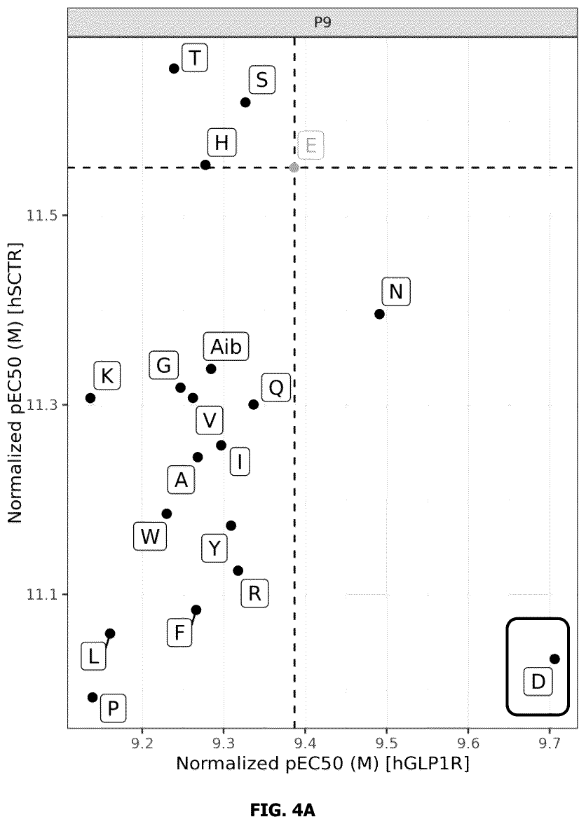

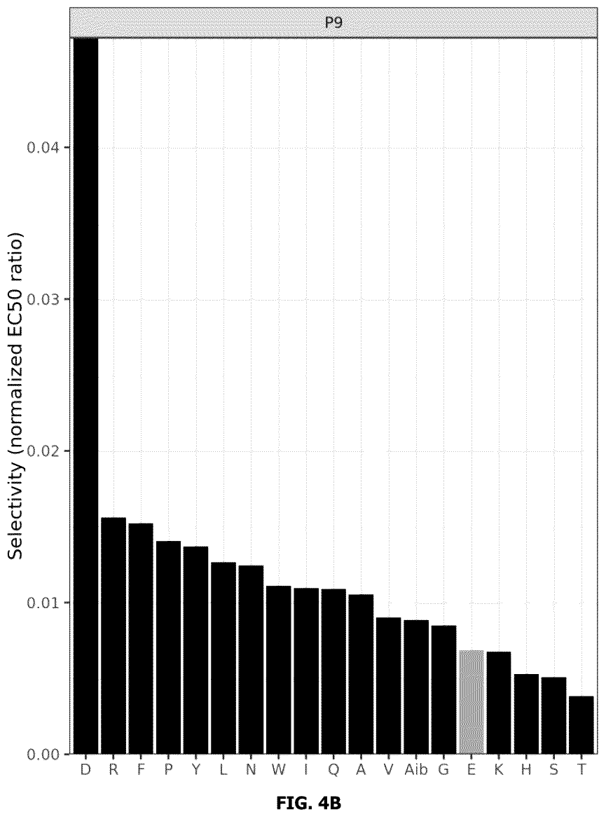

Position X⁹: Position X⁹ was identified as being important for both increasing hGLP1R potency and decreasing hSCTR potency to provide selective GLP1R agonists (see example 1). The deep mutational scan in example 2 revealed that the GLP-1 amino acid D in position X⁹ was superior in improving the hGLP1R potency and decreasing hSCTR potency compared to all of the amino acids tested (see example 2, FIGS. 4A and 4B). Thus, in the most preferred embodiment, X⁹ is selected as D to increase hGLP1R potency and decrease hSCTR potency of the SCT backbone.

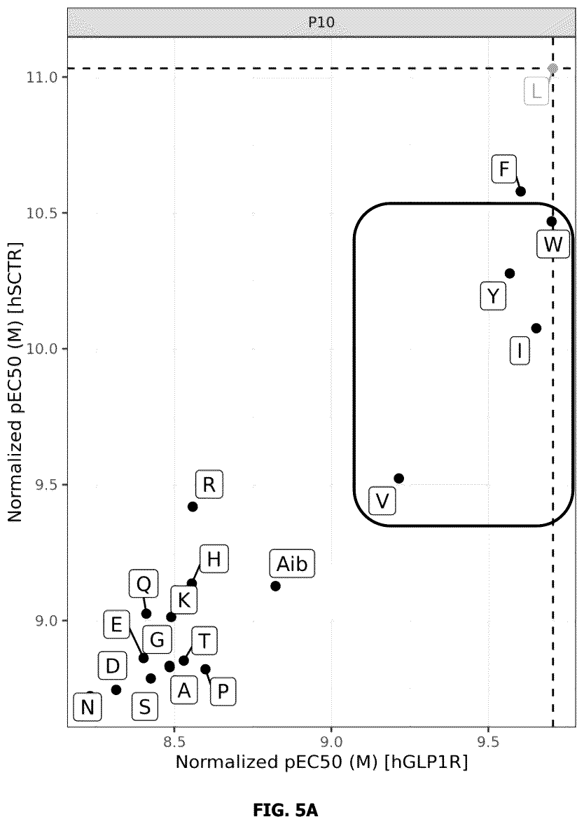

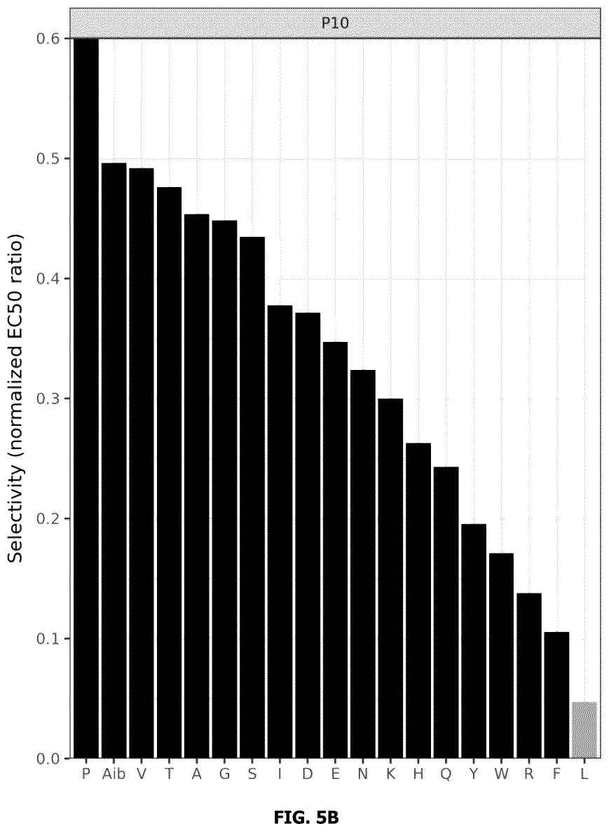

Position A¹⁰: Position X¹⁰ was identified as being important for decreasing hSCTR potency to provide selective GLP1R agonists (see example 1). The deep mutational scan revealed that in addition to the GLP-1 amino acid V (example 1), the non-GLP-1 amino acids P, Aib, V, T, A, G, S, I, D, E, N, K, H, Q, Y, W, R, F were found to decrease hSCTR potency compared to the amino acid L found in SCT in position X¹⁰ (see example 2, FIG. 5A). A decrease in hSCTR potency, in position X¹⁰, should preferably be accompanied by a minimum sacrifice of hGLP1R potency to achieve the highest selectivity for GLP1R over hSCTR. In FIG. 5B, the amino acids are ranked by selectivity (i.e. hSCTR EC₅₀/hGLP1R EC₅₀). The higher the EC₅₀ ratio is for a given amino acid, the higher the selectivity for GLP1R. Thus, when position X¹⁰ is used to maximise selectivity, X¹⁰ is preferably selected from P, Aib, V, T, A, G, S, I, D, E, N, or K (EC₅₀ ratio=0.3), more preferably X¹⁰ is selected from P, Aib, V, T, A, G, S, or I (EC₅₀ ratio=0.4), even more preferably X¹⁰ is selected from P, Aib, V, T, or A, most preferably X¹⁰ is selected from P, Aib, or V. As several positions are used to decrease hSCTR potency, it is possible to include amino acids in position X¹⁰ that prioritize high hGLP1R potency, even if less optimal from a selectivity point of view. Thus, as can be seen from FIG. 5A, position X¹⁰ may be selected as V, I, Y, or W to maintain maximum hGLP1R potency with varying reduction in hSCTR potency (see box in FIG. 5A). Thus, in a preferred embodiment, position X¹⁰ is selected as P, Aib, V, I, Y, or W to decrease the hSCTR potency of the SCT backbone. In a more preferred embodiment, X¹⁰ is selected as V, I, Y, or W to decrease the hSCTR potency of the SCT backbone. In an even more preferred embodiment, X¹⁰ is selected as V, I, or Y to decrease the hSCTR potency of the SCT backbone. In a most preferred embodiment, X¹⁰ is selected as V to decrease the hSCTR potency of the SCT backbone.

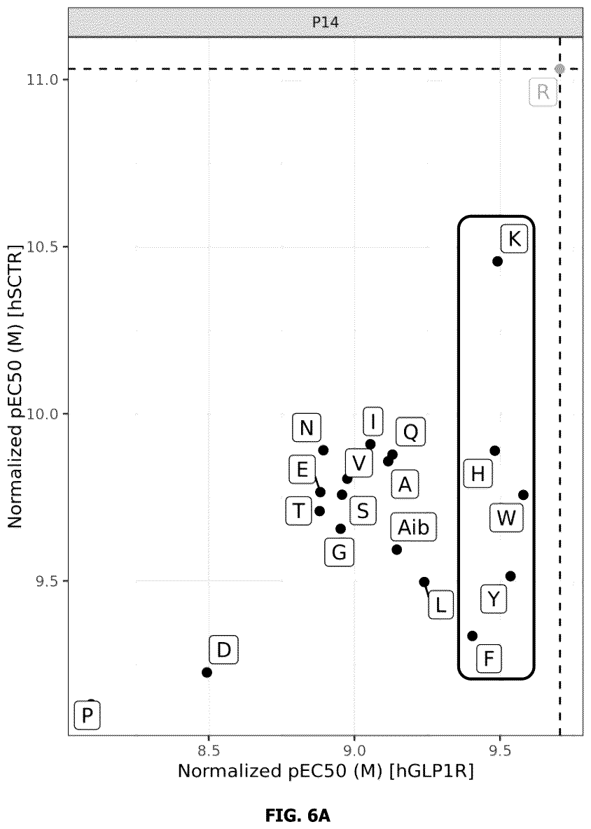

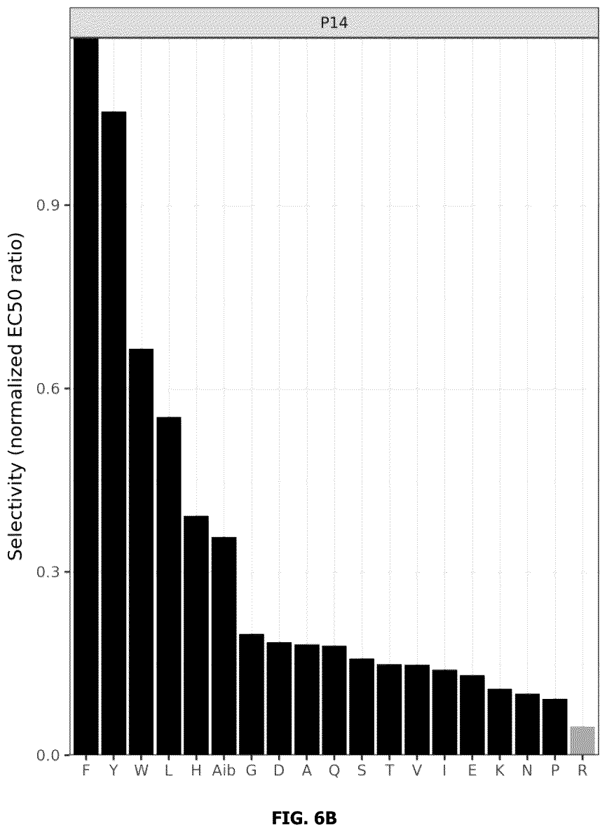

Position X¹⁴: Position X¹⁴ was identified as being important for decreasing hSCTR potency to provide selective GLP1R agonists (see example 1). The deep mutational scan revealed that in addition to the GLP-1 amino acid L (example 1), the non-GLP-1 amino acids F, Y, W, H, Aib, G, D, A, Q, S, T, V, I, E, K, N, and P were found to decrease hSCTR potency compared to the amino acid R found in SCT in position X¹⁴ (see example 2, FIG. 6A). A decrease in hSCTR potency, in position X¹⁴, should preferably be accompanied by a minimum sacrifice of hGLP1R potency to achieve the highest selectivity for GLP1R over hSCTR. In FIG. 6B, the amino acids are ranked by EC₅₀ ratio (i.e. hSCTR EC₅₀/hGLP1R EC₅₀). The higher the EC₅₀ ratio is for a given amino acid, the higher the selectivity is for GLP1R. Thus, when position X¹⁴ is used to maximise selectivity, X¹⁴ is preferably selected from F, Y, W, L, H, or Aib (EC₅₀ ratio cut off=0.3), more preferably X¹⁴ is selected from F, Y, W, or L (EC₅₀ ratio cut off=0.4), even more preferably X¹⁴ is selected from F, Y, or W (EC₅₀ ratio cut off=0.6), most preferably X¹⁴ is selected from F or Y (EC₅₀ ratio=0.9). As several positions are used to decrease hSCTR potency, it is possible to include amino acids in position X¹⁴ that prioritize high hGLP1R potency, even if less optimal from a selectivity point of view. Thus, as can be seen from FIG. 6A, position X¹⁴ may be selected as F, Y, W, H, or K to maintain maximum hGLP1R potency with varying reduction in hSCTR potency (see box in FIG. 6A). Thus, in an embodiment, position X¹⁴ is selected as F, Y, W, L, H, Aib or K to decrease the hSCTR potency of the SCT backbone. In a preferred embodiment, X¹⁴ is selected as F, Y, W, L, H, or K to decrease the hSCTR potency of the SCT backbone. In a more preferred embodiment, X¹⁴ is selected as F, Y, W, L, or K to decrease the hSCTR potency of the SCT backbone. In the most preferred embodiment, X¹⁴ is selected as L or K.

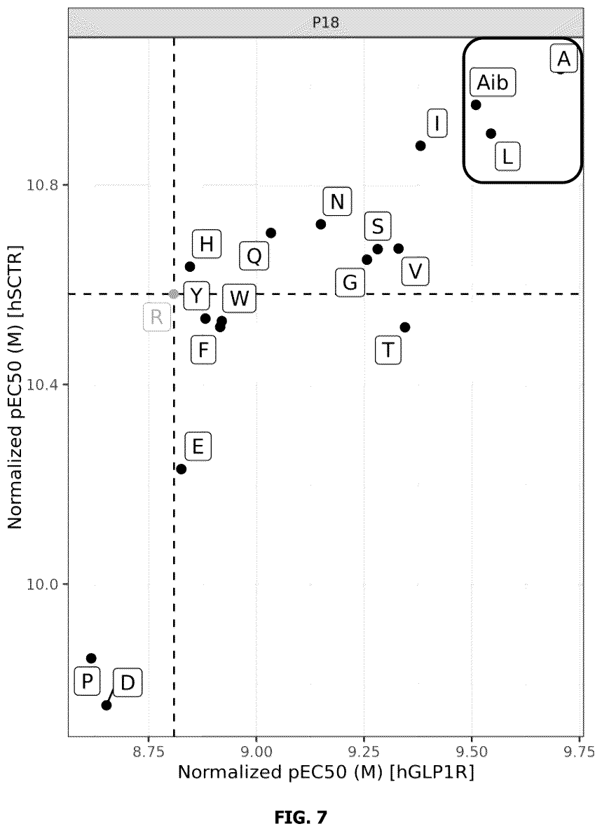

Position X¹⁸: Position X¹⁸ was identified as being important for increasing hGLP1R potency (see example 1). The deep mutational scan revealed that the tested amino acids A, Aib, and L in position X¹⁸ resulted in the highest potency compared to the other amino acids tested (see example 2, FIG. 7). Thus, in an embodiment, position X¹⁸ is selected as N, G, S, V, T, I, A, Aib, or L. In a preferred embodiment, position X¹⁸ is selected as S, V, T, I, A, Aib, or L. In a more preferred embodiment, position X¹⁸ is selected as T, I, A, Aib, or L. In an even more preferred embodiment, position X¹⁸ is selected as A, Aib, or L to increase hGLP1R potency of the SCT backbone. In the most preferred embodiment, position X¹⁸ is selected as Aib.

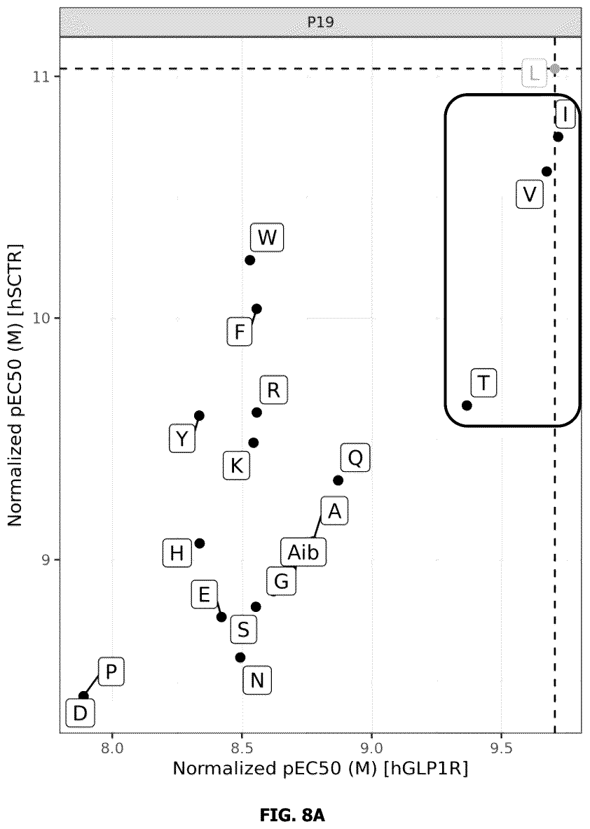

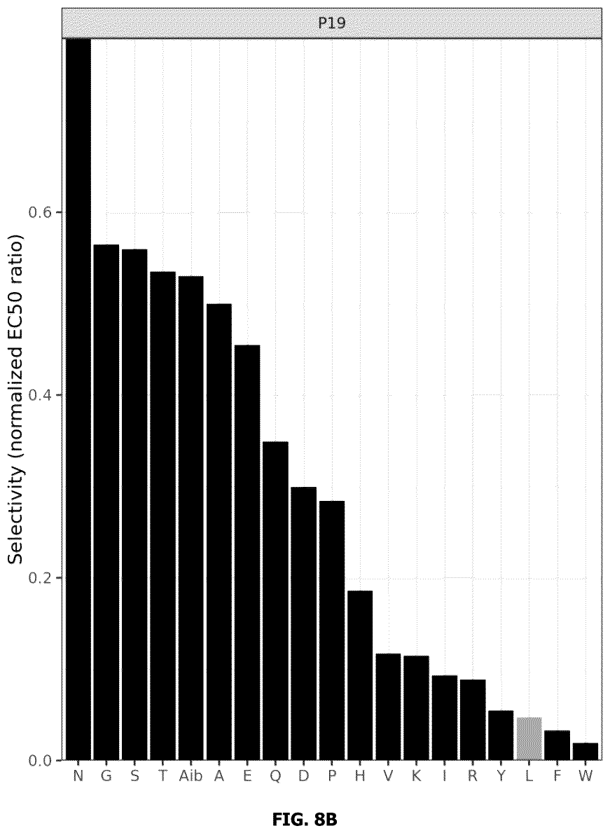

Position X¹⁹: Position X¹⁹ was identified as being important for decreasing hSCTR potency to provide selective GLP1R agonists (see example 1). The deep mutational scan revealed that in addition to the GLP-1 amino acid A (example 1), all of the non-GLP-1 amino acids I, V, W, F, Y, R, T, K, Q, H, Aib, G, E, S, N, P, and D were found to decrease hSCTR potency compared to the amino acid L found in SCT in position X¹⁹ (see example 2, FIG. 8A). A decrease in hSCTR potency, in position X¹⁹, should preferably be accompanied by a minimum sacrifice of hGLP1R potency to achieve the highest selectivity for GLP1R over hSCTR. In FIG. 8B, the amino acids are ranked by selectivity (i.e. hSCTR EC₅₀/hGLP1R EC₅₀). The higher the EC₅₀ ratio is for a given amino acid, the higher the selectivity is for GLP1R. Thus, when position X¹⁹ is used to maximise selectivity, X¹⁹ is preferably selected from N, G, S, T, Aib, A, E, Q, D, or P (EC₅₀ ratio cut off=0.2), more preferably X¹⁹ is selected from N, G, S, T, Aib, A, E, or Q (EC₅₀ ratio cut off=0.3), even more preferably X¹⁹ is selected from N, G, S, T, Aib, A or E (EC₅₀ ratio cut off=0.4), yet more preferably X¹⁹ is selected from N, G, S, T, Aib, or A (EC₅₀ ratio cut off=0.5), most preferably X¹⁹ is selected from N (EC₅₀ ratio=0.6). As several positions are used to decrease hSCTR potency, it is possible to include amino acids in position X¹⁹ that prioritize high hGLP1R potency, even if less optimal from a selectivity point of view. Thus, as can be seen from FIG. 8A, position X¹⁹ may be selected as I, V, and T to maintain maximum hGLP1R potency with varying degree of reduction in hSCTR potency (see box in FIG. 8A). Thus, in a more preferred embodiment, position X¹⁹ is selected as L, N, G, S, T, Aib, A, E, Q, I, or V. The inventors identified that the peptides performed better in in vivo models when X¹⁹ was selected as L. Thus, in a most preferred embodiment, X¹⁹ is selected as L.

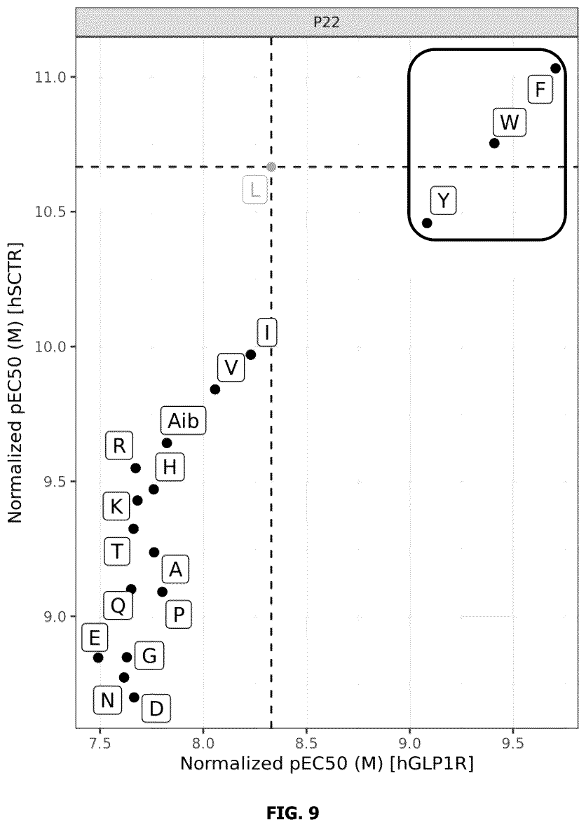

Position X²²: Position X²² was identified as being important for hGLP1R potency (see example 1). The deep mutational scan revealed that the amino acids F, W, and Y resulted in the highest hGLP1R potency in position X²² compared to the amino acid L found in SCT in position X²² (see FIG. 9). Thus, in a more preferred embodiment, position X²² is selected as F, W, or Y to increase hGLP1R potency of the SCT backbone. In the most preferred embodiment, X²² is selected as F.

### Optional Positions in SCT Backbone for Providing Potent and Selective GLP1R Agonists

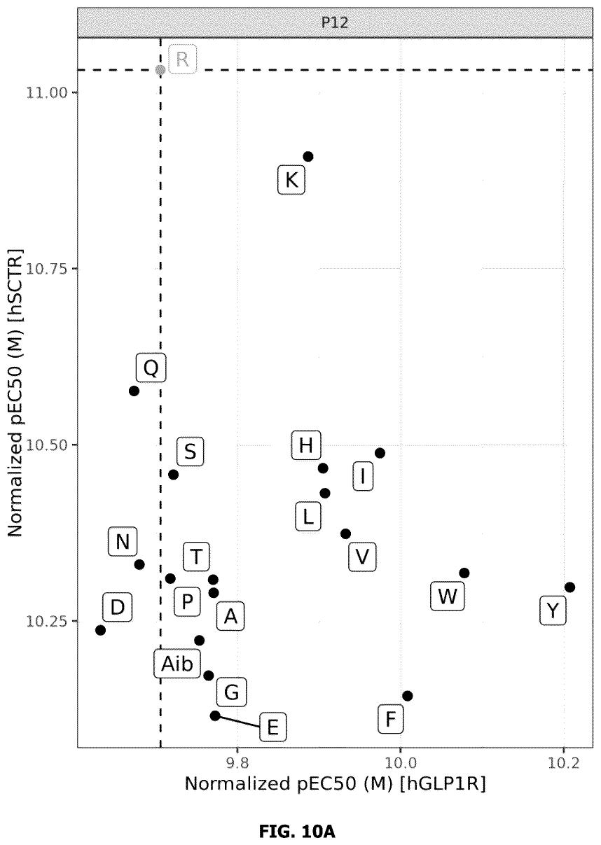

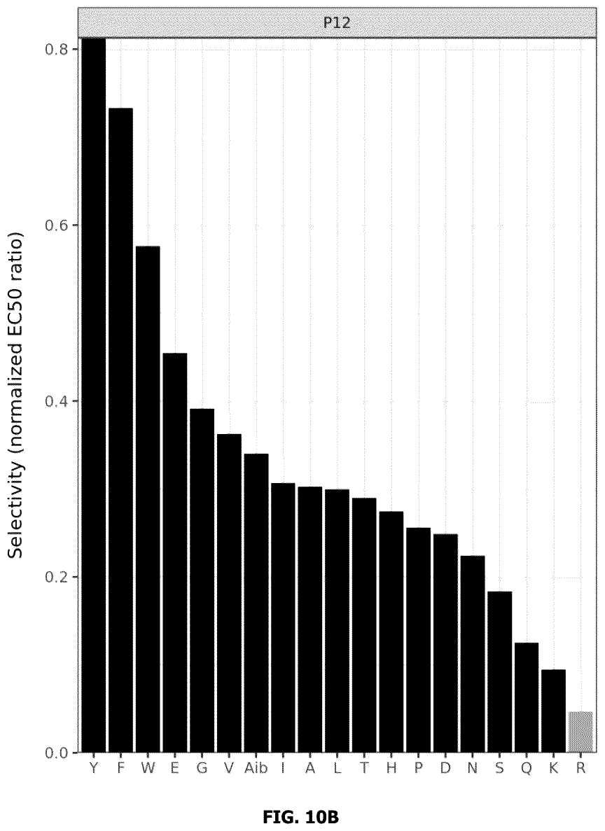

Position X¹²: The deep mutational scan revealed that position X¹² provided a further residue for increasing the GLP1R selectivity of the peptides. As can be seen from example 2, the amino acids Y, F, W, E, G, V, Aib, I, A, L, T, H, P, D, N, S, Q, and K decreased hSCTR potency compared to the amino acid R found in SCT in position X¹², with a minor reduction in hGLP1R potency or even a slight improvement in hGLP1R potency (see FIG. 10A). FIG. 10B ranks the amino acids by EC₅₀ ratio (i.e. hSCTR EC₅₀/hGLP1R EC₅₀). The higher the EC₅₀ ratio of a given amino acid, the higher the selectivity. Thus, X¹² is preferably selected from Y, F, W, E, G, V, Aib, I, A, L, T, H, P, D, or N (EC₅₀ ratio cut off=0.2), more preferably X¹² is selected from Y, F, W, E, G, V, or Aib (EC₅₀ ratio cut off=0.3), even more preferably X¹² is selected from Y, F, W, or E (EC₅₀ ratio cut off=0.4), yet more preferably X¹² is selected from Y, F or W (EC₅₀ ratio cut off=0.5), even more preferably X¹² is selected from Y or F (EC₅₀ ratio=0.6) to decrease the hSCTR potency of the SCT backbone. In the most preferred embodiment, X¹² is selected from Y. Table 4 shows the effect of introducing Y, F, W, E, G, V, or Aib in comparison with the closest prior art peptide GUB06-046.

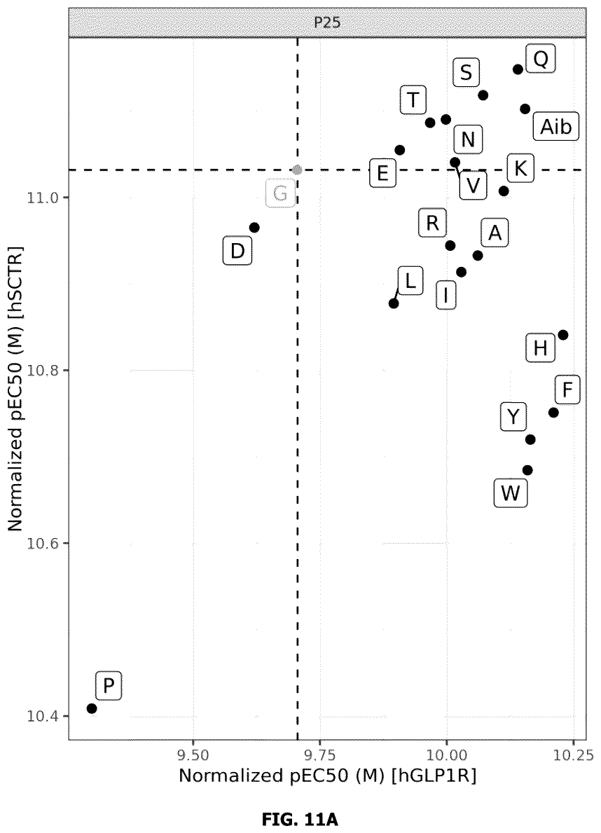

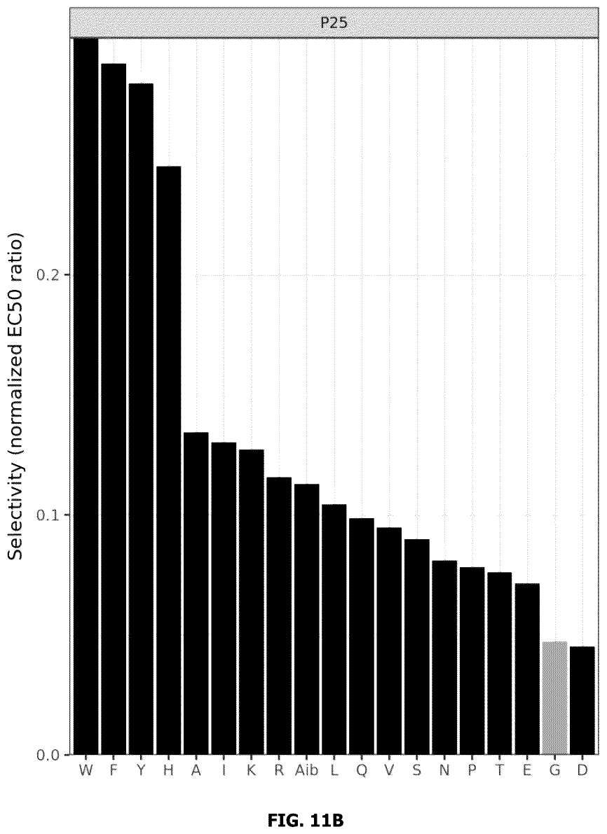

Position X²⁵: The deep mutational scan revealed that position X²⁵ provided a further residue for increasing the GLP1R selectivity of the peptides. As can be seen from example 2, the amino acids S, Q, T, N, Aib, E, V, K, R, A, I, L, H, F, Y, and W was found to increase hGLP1R potency compared to the amino acid G found in SCT in position X²⁵, with varying effect on hSCTR potency (see FIG. 11A). FIG. 11B ranks the amino acids by EC₅₀ ratio (i.e. hSCTR EC₅₀/hGLP1R EC₅₀). The higher the EC₅₀ ratio of a given amino acid, the higher the selectivity. Thus, X²⁵ is preferably selected from W, F, Y, H, A, I, K, R, or Aib (EC₅₀ ratio cut off=0.1). Most preferably X²⁵ is selected from W, F, Y, H, or K (EC₅₀ ratio cut off=0.2). In the most preferred embodiment, X²⁵ is selected as H or K.

Positions for Introducing Lipids without Compromising hGLP1R Potency

Optimal lipidation sites were identified by introducing lipidated lysine residues in each of the positions of native GLP-1 (see example 3). As can be seen from FIG. 12, position X¹⁴ and X²⁵ were identified as the best lipidation sites without compromising the hGLP1R potency. Thus, in a most preferred embodiment of the invention, X¹⁴ or X²⁵ is selected as K, in order to lipidate the peptides to improve the PK properties.

Positions for Improving Solubility without Compromising hGLP1R Potency

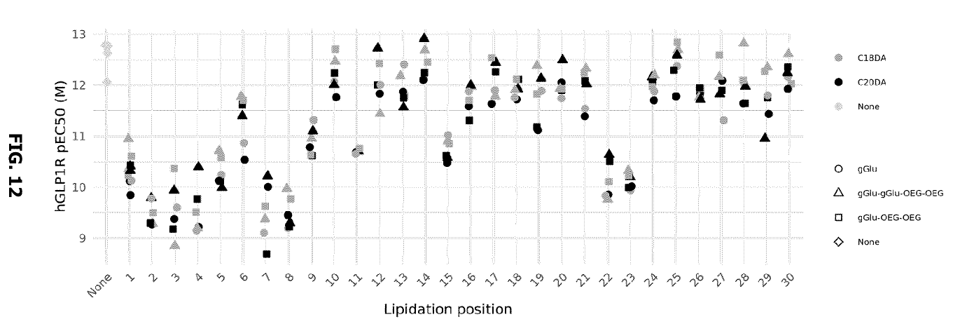

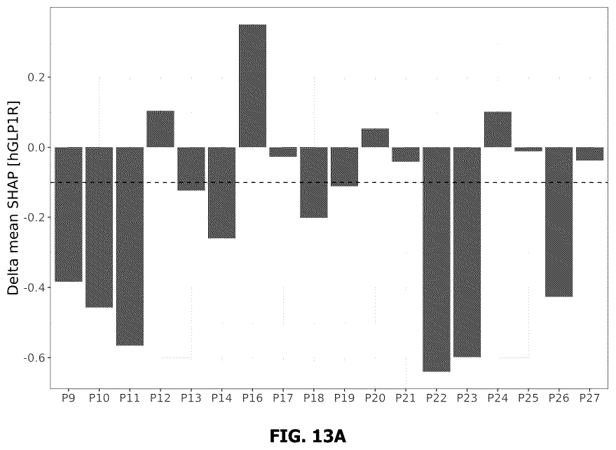

Positions tolerating glutamate (E) residues were identified in order to improve the solubility of the peptides (see example 4). As shown in FIG. 13A, positions X¹², X¹⁶, X¹⁷, X²⁰, X²¹, X²⁴, X²⁵, and X²⁷ were identified as suitable positions for increasing the solubility of the peptides by introducing glutamate with minimal adverse effect on GLP1R potency. Thus, in an embodiment of the invention, one or more of the positions X¹², X¹⁶, X¹⁷, X²⁰, X²¹, X²⁴, X²⁵, and X²⁷ is/are selected as E. In a preferred embodiment, one or more of the positions X¹², X¹⁶, X²⁰, or X²⁴, is selected as E. In particular, X¹⁶ and X²⁴ were identified as optimal for introducing glutamate residues with a slightly positive effect on GLP1R potency (see FIG. 13A). Thus, in a highly preferred embodiment, X¹⁶ and/or X²⁴ is selected as E. In a most preferred embodiment, X¹⁶ and X²⁴ are selected as E.

Positions for Improving Physical Stability without Compromising hGLP1R Potency

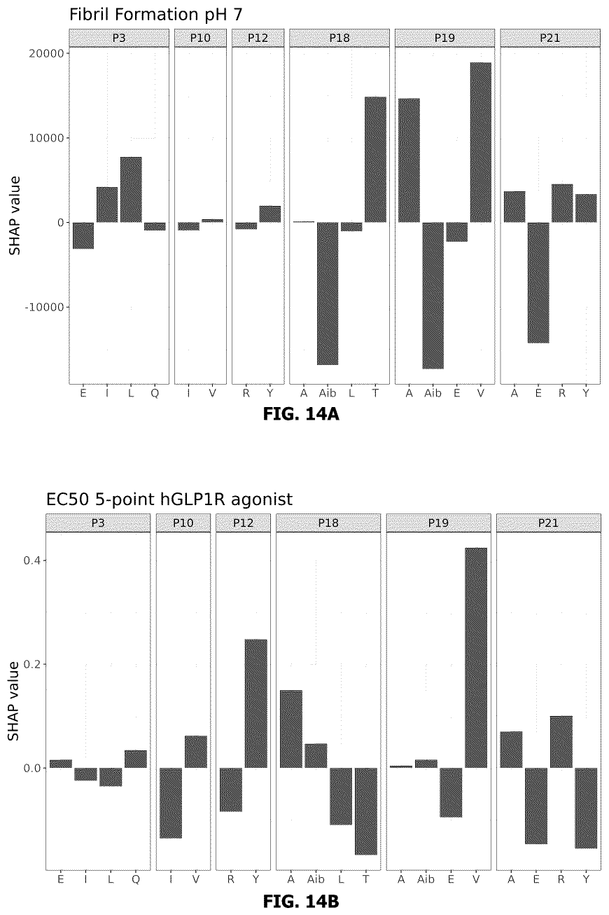

As can be seen in FIG. 14A, Aib in position X¹⁸ and/or X¹⁹ decreased the tendency of the peptides to fibrillate at pH 7. Likewise, E in position X²¹ decreased the tendency of the peptides to fibrillate at pH 7. As can be seen from FIG. 14B, Aib in position X¹⁸ and/or X¹⁹ did not have any adverse effect on GLP1R potency, whereas E in position X²¹ had a slightly negative effect on GLP1R potency. Thus, in a preferred embodiment, one or more of the positions X¹⁸, X¹⁹ and/or X²¹ is/are used to decrease the peptides tendency to fibrillate by introducing Aib in X¹⁸, Aib in X¹⁹ and/or E in X²¹. In a preferred embodiment, one of the positions X¹⁸, X¹⁹ or X²¹ is used to decrease the peptides tendency to fibrillate by introducing Aib in X¹⁸, Aib in X¹⁹ or E in X²¹. In the most preferred embodiment, X¹⁸ is selected as Aib to decrease the peptides tendency to fibrillate.

In a first aspect, the invention relates to a polypeptide or a pharmaceutically acceptable salt thereof comprising 27 amino acid residues (X¹-X²⁷), said polypeptide having at least 50% identity to human secretin (SCT), wherein
X² is selected as A, Aib, or G;
X³ is selected as I, H, L, W, V, Y, E or Q;
X⁹ is D;
X¹⁴ is selected as F, Y, W, L, H, Aib or K
X¹⁸ is selected as A, Aib, or L;
X¹⁹ is selected as L, N, G, S, T, Aib, A, E, Q, I or V.
X²² is selected as F, W or Y;
characterized in that X¹⁰ is selected as P, Aib, V, I, Y, or W and in that said polypeptide has a SCTR EC₅₀/GLP1R EC₅₀ ratio of at least 100.

### Preferred Embodiments of the First Aspect

In a preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F, W, or Y. In another preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as A, Aib, or L; X¹⁹ is selected as L; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as A, Aib, or L; X¹⁹ is selected as L; X²² is selected as F. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as Aib; X¹⁹ is selected as L; X²² is selected as F, W, or Y. In another preferred embodiment, X² is selected as A, Aib, or G; X³ is selected as E; X⁹ is selected as D; X¹⁰ is selected as V, I, Y, or W; X¹⁴ is selected as L or K; X¹⁸ is selected as Aib; X¹⁹ is selected as L; X²² is selected as F. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In another preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F. In another preferred embodiment, X² is selected as A, Aib, or G; X³ is selected as E; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F, W, or Y. In another preferred embodiment, X² is selected as A, Aib, or G; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F. In another preferred embodiment, X² is selected as A, Aib, or G; X³ is selected as E; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as A, Aib, or G; X³ is selected as E; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as A, Aib, or G; X³ is selected as E; X⁹ is selected as D; X¹⁰ is selected as V; X¹⁴ is selected as L or K; X¹⁸ is selected as Aib; X¹⁹ is selected as L; X²² is selected as F, W, or Y. In another preferred embodiment, X² is selected as A, Aib, or G; X³ is selected as E; X⁹ is selected as D; X¹⁰ is selected as V; X¹⁴ is selected as L or K; X¹⁸ is selected as Aib; X¹⁹ is selected as L; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as A, Aib, or L; X¹⁹ is selected as L; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V, I, Y, or W; X¹⁴ is selected as L or K; X¹⁸ is selected as Aib; X¹⁹ is selected as L; X²² is selected as F. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F, W, or Y. In another preferred embodiment, X² is selected as Aib; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F, W, or Y. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as A, Aib, or L; X¹⁹ is selected as L; X²² is selected as F. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F, W, or Y. In another preferred embodiment, X² is selected as Aib; X³ is selected as E; X⁹ is selected as D; X¹⁰ is selected as V, I, Y, or W; X¹⁴ is selected as F, Y, W, L, or K; X¹⁸ is selected as Aib; X¹⁹ is selected as L; X²² is selected as F. In another preferred embodiment, X² is selected as Aib; X³ is selected as E; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V, I, Y, or W; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V, I, Y, or W; X¹⁴ is selected as L or K; X¹⁸ is selected as A, Aib, or L; X¹⁹ is selected as L; X²² is selected as F, W, or Y. In another preferred embodiment, X² is selected as Aib; X³ is selected as E; X⁹ is selected as D; X¹⁰ is selected as V, I, Y, or W; X¹⁴ is selected as L or K; X¹⁸ is selected as A, Aib, or L; X¹⁹ is selected as L; X²² is selected as F. In another preferred embodiment, X² is selected as Aib; X³ is selected as E; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V, I, Y, or W; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V, I, Y, or W; X¹⁴ is selected as L or K; X¹⁸ is selected as Aib; X¹⁹ is selected as L; X²² is selected as F, W, or Y. In another preferred embodiment, X² is selected as Aib; X³ is selected as E; X⁹ is selected as D; X¹⁰ is selected as V, I, Y, or W; X¹⁴ is selected as L or K; X¹⁸ is selected as Aib; X¹⁹ is selected as L; X²² is selected as F. In another preferred embodiment, X² is selected as Aib; X³ is selected as E; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V; X¹⁴ is selected as F, Y, W, L, or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In another preferred embodiment, X² is selected as Aib; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In another preferred embodiment, X² is selected as Aib; X³ is selected as E; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V; X¹⁴ is selected as F, Y, W, L, or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In another preferred embodiment, X² is selected as Aib; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F. In another preferred embodiment, X² is selected as Aib; X³ is selected as E; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V; X¹⁴ is selected as L or K; X¹⁸ is selected as A, Aib, or L; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F, W, or Y. In another preferred embodiment, X² is selected as Aib; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F. In another preferred embodiment, X² is selected as Aib; X³ is selected as E; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F, W, or Y. In another preferred embodiment, X² is selected as Aib; X³ is selected as E; X⁹ is selected as D; X¹⁰ is selected as V; X¹⁴ is selected as L or K; X¹⁸ is selected as Aib; X¹⁹ is selected as L, N, G, S, T, Aib, A, E, Q, I, or V; X²² is selected as F. In another preferred embodiment, X² is selected as Aib; X³ is selected as E; X⁹ is selected as D; X¹⁰ is selected as V; X¹⁴ is selected as L or K; X¹⁸ is selected as Aib; X¹⁹ is selected as L; X²² is selected as F, W, or Y. In another preferred embodiment, X² is selected as Aib; X³ is selected as E; X⁹ is selected as D; X¹⁰ is selected as V; X¹⁴ is selected as L or K; X¹⁸ is selected as Aib; X¹⁹ is selected as L; X²² is selected as F.

In a highly preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In a highly preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In a highly preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y, In a highly preferred embodiment, X² is selected as A, Aib, or G; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F. In a highly preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F, W, or Y. In a highly preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F. In a highly preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F, W, or Y. In a highly preferred embodiment, X² is selected as A, Aib, or G; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F. In a highly preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In a highly preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In a highly preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In a highly preferred embodiment, X² is selected as A, Aib, or G; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F. In a highly preferred embodiment, X² is selected as A, Aib, or G; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F, W, or Y. In a highly preferred embodiment, X² is selected as A, Aib, or G; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F. In a highly preferred embodiment, X² is selected as A, Aib, or G; X³ is selected as E; X⁹ is selected as D; X¹⁰ is selected as V; X¹⁴ is selected as L or K; X¹⁸ is selected as Aib; X¹⁹ is selected as L; X²² is selected as F, W, or Y. In a highly preferred embodiment, X² is selected as A, Aib, or G; X³ is selected as E; X⁹ is selected as D; X¹⁰ is selected as V; X¹⁴ is selected as L or K; X¹⁸ is selected as Aib; X¹⁹ is selected as L; X²² is selected as F. In a highly preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In a highly preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In a highly preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In a highly preferred embodiment, X² is selected as Aib; X³ is selected as I, H, L, E, or Q; X⁹ is selected as D; X¹⁰ is selected as V; X¹⁴ is selected as F, Y, W, L, or K; X¹⁸ is selected as Aib; X¹⁹ is selected as L; X²² is selected as F. In a highly preferred embodiment, X² is selected as Aib; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F, W, or Y. In a highly preferred embodiment, X² is selected as Aib; X³ is selected as I, H, L, E, or Q; X⁹ is selected as D; X¹⁰ is selected as V; X¹⁴ is selected as L or K; X¹⁸ is selected as A, Aib, or L; X¹⁹ is selected as L; X²² is selected as F. In a highly preferred embodiment, X² is selected as Aib; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F, W, or Y. In a highly preferred embodiment, X² is selected as Aib; X³ is selected as I, H, L, E, or Q; X⁹ is selected as D; X¹⁰ is selected as V; X¹⁴ is selected as L or K; X¹⁸ is selected as Aib; X¹⁹ is selected as L; X²² is selected as F. In a highly preferred embodiment, X² is selected as Aib; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F, W, or Y. In a highly preferred embodiment, X² is selected as Aib; X³ is selected as E; X⁹ is selected as D; X¹⁰ is selected as V; X¹⁴ is selected as F, Y, W, L, or K; X¹⁸ is selected as A, Aib, or L; X¹⁹ is selected as L; X²² is selected as F. In a highly preferred embodiment, X² is selected as Aib; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F, W, or Y. In a highly preferred embodiment, X² is selected as Aib; X³ is selected as E; X⁹ is selected as D; X¹⁰ is selected as V; X¹⁴ is selected as F, Y, W, L, or K; X¹⁸ is selected as Aib; X¹⁹ is selected as L; X²² is selected as F. In a highly preferred embodiment, X² is selected as Aib; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F, W, or Y. In a highly preferred embodiment, X² is selected as Aib; X³ is selected as E; X⁹ is selected as D; X¹⁰ is selected as V; X¹⁴ is selected as L or K; X¹⁸ is selected as A, Aib, or L; X¹⁹ is selected as L; X²² is selected as F. In a highly preferred embodiment, X² is selected as Aib; X³ is selected as E; X⁹ is selected as D; X¹⁰ is selected as V; X¹⁴ is selected as L or K; X¹⁸ is selected as Aib; X¹⁹ is selected as L; X²² is selected as F, W, or Y. In a highly preferred embodiment, X² is selected as Aib; X³ is selected as E; X⁹ is selected as D; X¹⁰ is selected as V; X¹⁴ is selected as L or K; X¹⁸ is selected as Aib; X¹⁹ is selected as L; X²² is selected as F.

In a most preferred embodiment of the first aspect, X² is selected as Aib; X³ is selected as E; X⁹ is selected as D; X¹⁰ is selected as V; X¹⁴ is selected as L or K; X¹⁸ is selected as Aib; X¹⁹ is selected as L; X²² is selected as F.

In any of the above embodiments of the first aspect, X₁₂ is most preferably selected as Y, and X²⁵ is most preferably selected as K or H.

In any of the embodiments, wherein X¹⁴ is selected as K, this lysine residue is used as lipidation site when the peptides are lipidated. Likewise, in any of the embodiments, wherein X²⁵ is selected as K, this lysine residue is used as lipidation site when the peptides are lipidated. It is most preferred that only one of X¹⁴ or X²⁵ is selected as K such that the peptides only comprise a single lysine residue that is lipidated. Thus, in any of the above embodiments, when X¹⁴ is selected as K for providing a lipidation site, X²⁵ is most preferably selected as H. Likewise, in any of the above embodiments, when X²⁵ is selected as K for providing a lipidation site, X¹⁴ is most preferably selected as L.

In a second aspect, the invention relates to a polypeptide or a pharmaceutically acceptable salt thereof comprising the structure of Formula (I),

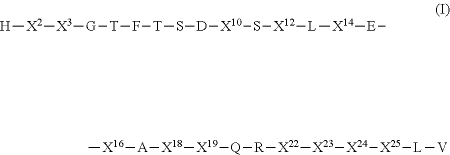

wherein,
X² is selected as A, Aib, or G;
X³ is selected as I, H, L, W, V, Y, E or Q;
X¹² is selected as Y, F, W, E, G, V, Aib, I, A, L, T, H, P, D, N, or R;
X¹⁴ is selected as F, Y, W, L, H, Aib or K
X¹⁶ is selected as E, G, T, F, L, or Aib;
X¹⁸ is selected as A, Aib, or L;
X¹⁹ is selected as L, N, G, S, T, Aib, A, E, Q, I or V;
X²² is selected as F, W or Y;
X²³ is selected as L or I;
X²⁴ is selected as E or Q;
X²⁵ is selected as W, F, Y, H, K or E;
characterized in that X¹⁰ is selected as P, Aib, V, I, Y, or W.

### Embodiments of the Second Aspect

It should be appreciated that each of the amino acid positions X², X³, X¹⁰, X¹², X¹⁴, X¹⁶, X¹⁸, X¹⁹, X²², X²³, X²⁴, and X²⁵ is selected as the amino acid or one of the amino acid groups described above under the headings “Essential positions in SCT backbone for providing potent and selective GLP1R agonists”, “Optional positions in SCT backbone for providing potent and selective GLP1R agonists”; “Positions for introducing lipids without compromising hGLP1R potency” and “Positions for improving solubility without compromising hGLP1R potency”. Thus, any combination of the various embodiments under these headings are contemplated irrespective of whether the embodiment is “preferred”, “more preferred” or “most preferred”.

### Preferred Embodiments of the Second Aspect

The listed embodiments of aspect 1 specifying preferred amino acids for positions X², X³, X¹⁰, X¹⁴, X¹⁸, X¹⁹, and X²² equally apply to the second aspect. Thus, any of the embodiments mentioned under the heading “Preferred embodiments of the first aspect” also apply to the second aspect, with the further addition that for each embodiment mentioned, X¹² is preferably selected from Y, F, W, E, G, V, or Aib, even more preferably, X¹² is selected from Y, F, W, or E, yet more preferably, X¹² is selected from Y, F or W, most preferably, X¹² is selected as Y; X¹⁶ is selected as E, G, T, F, L, or Aib, most preferably E; X²³ is selected as L or I, most preferably L; X²⁴ is selected as E or Q, most preferably E.

In another preferred embodiment, X² is selected as A, Aib, or G; X³ is selected as I, H, L, E, or Q; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as I, H, L, E, or Q; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as A, Aib, or G; X³ is selected as I, H, L, E, or Q; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as I, H, L, E, or Q; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as A, Aib, or G; X³ is selected as I, H, L, E, or Q; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as I, H, L, E, or Q; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as A, Aib, or G; X³ is selected as E; X¹⁰ is selected as V, I, Y, or W; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as E; X¹⁰ is selected as V, I, Y, or W; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as A, Aib, or G; X³ is selected as E; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as E; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as A, Aib, or G; X³ is selected as E; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as E; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as A, Aib, or G; X³ is selected as E; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as A, Aib, or G; X³ is selected as E; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as Aib; X³ is selected as I, H, L, E, or Q; X¹⁰ is selected as V, I, Y, or W; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as I, H, L, E, or Q; X¹⁰ is selected as V, I, Y, or W; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as Aib; X³ is selected as I, H, L, E, or Q; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as I, H, L, E, or Q; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as Aib; X³ is selected as I, H, L, E, or Q; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as I, H, L, E, or Q; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as Aib; X³ is selected as I, H, L, E, or Q; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as I, H, L, E, or Q; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as Aib; X³ is selected as E; X¹⁰ is selected as V, I, Y, or W; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K. In another preferred embodiment, X² is selected as Aib; X³ is selected as E; X¹⁰ is selected as V, I, Y, or W; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as Aib; X³ is selected as E; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as E; X¹⁰ is selected as V, I, Y, or W; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

In another preferred embodiment, X² is selected as Aib; X³ is selected as E; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as E; X¹⁰ is selected as V; X¹² is selected as Y, F or V; X¹⁴ is selected as L, or K; X¹⁶ is selected as E; X¹⁸ is selected A, Aib, or L; X¹⁹ is selected as L; X²² is selected as F; X²³ is selected as L; X²⁴ is selected as E; X²⁵ is selected as H or K.

In another preferred embodiment, X² is selected as Aib; X³ is selected as E; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F, W, or Y; X²³ is selected as L; X²⁴ is selected as E; X²⁵ is selected as H or K.

In another preferred embodiment, X² is selected as Aib; X³ is selected as E; X¹⁰ is selected as V; X¹² is selected as Y, F or V; X¹⁴ is selected as L, or K; X¹⁶ is selected as E; X¹⁸ is selected as Aib; X¹⁹ is selected as L; X²² is selected as F; X²³ is selected as L; X²⁴ is selected as E; X²⁵ is selected as H or K.

As shown in Tables 1 and 2 herein, the exemplified peptides have a SCTR EC₅₀/GLP1R EC₅₀ ratio of at least 100, most of the peptides much higher. As can be seen from Table 3, in comparison with the prior art peptide GUB06-046, the amino acids P, Aib, V, I, Y, or W in position X¹⁰ result in an increase in selectivity. Likewise, as can be seen from Table 4, in comparison with the prior art peptide GUB06-046, the amino acids Y, F, W, E, G, V, or Aib in position X¹² result in an increase in selectivity. Thus, in a most preferred embodiment, the polypeptides are characterized in said polypeptides having a SCTR EC₅₀/GLP1R EC₅₀ ratio of at least 100.

In any of the embodiments of the second aspect, wherein X¹⁴ is selected as K, this lysine residue is used as lipidation site when the peptides are lipidated. Likewise, in any of the embodiments of the second aspect, wherein X²⁵ is selected as K, this lysine residue is used as lipidation site when the peptides are lipidated. It is most preferred that only one of X¹⁴ or X²⁵ is selected as K such that the peptides only comprise a single lysine residue that is lipidated. Thus, in any of the embodiments of the second aspect, when X¹⁴ is selected as K for providing a lipidation site, X²⁵ is most preferably selected as H. Likewise, in any of the embodiments of the second aspect, when X²⁵ is selected as K for providing a lipidation site, X¹⁴ is most preferably selected as L. Most, preferably X¹⁴ is selected as K and used as lipidation site.

Tables 1 and 2 exemplify selective GLP1R agonists according to the invention having the amino acid residues in positions X², X³, X⁹, X¹⁰, X¹⁴, X¹⁸, X¹⁹, and X²² capable of converting SCT into selective GLP1R agonists. The last row of Table 1 summarizes the exemplified amino acids in the identified positions.

### Extended GLP1R Agonists Based on SCT

As illustrated in example 7 and Table 2, the GLP1R agonists according to the invention may be extended to the full length of native GLP-1 (i.e. 30 amino acid residues). Thus, in any of aspect 1, 2 or 3 or in any of their embodiments, the peptides may be extended with 1 (i.e. X²⁸), 2 (i.e. X²⁸-X²⁹), or 3 (i.e. X²⁸-X²⁹-X³⁰) additional amino acid residues. When extended, the amino acid in position X²⁸ may be selected from any of the amino acids Aib, A, D, E, F, G, H, I, K, L, N, P, Q, R, S, T, V, W, or Y. However, it is less preferred that X²⁸ is selected as F or P. Preferably, X²⁸ is selected as A or E. Most preferably, X²⁸ is selected as E. The amino acid in position X²⁹ may be selected of any of the amino acids Aib, A, D, E, F, G, H, I, K, L, N, P, Q, R, S, T, V, W, or Y. However, it is most preferred that X²⁹ is selected as A, Aib, or G. The amino acid in position X³⁰ may be selected of any of the amino acids Aib, A, D, E, F, G, H, I, K, L, N, P, Q, R, S, T, V, W, or Y. However, it is most preferred that X³⁰ is selected as A or E. Thus, in a highly preferred embodiment, when the GLP1R agonists according to the invention comprise 30 amino acid residues, X²⁸ is selected A or E; X²⁹ is selected as A, Aib, or G; and X³⁰ is selected as A or E. In a most preferred embodiment, when the GLP1R agonists according to the invention comprise 30 amino acid residues, X²⁸ is selected as E; X²⁹ is selected as A; and X³⁰ is selected as A.

### Medical Use

In a fourth aspect, the present invention relates to a peptide according to any of the previous aspects or embodiments for use as a medicament, in particular for use as a medicament in the treatment of obesity, diabetes and/or renal diseases, and/or non-alcoholic steatohepatitis (NASH), most preferably obesity and/or diabetes.

### EXPERIMENTAL SECTION

### General Procedure for Peptide Synthesis

The peptides were synthesized using a SyroII fully automated parallel peptide synthesizer (MultiSynTech GmbH, Germany), equipped with heating block, on Tentagel S RAM with a loading of 0.23-0.25 mmol/g (Rapp polymer GmbH, Germany). N^{α}-Fmoc deprotection was performed in two stages by treating the resin with 40% piperidine/DMF (0.2 M HOBt (1-hydroxybenzotriazole)) for 3 min at 45° C. followed by 20% piperidine/DMF (0.1 M HOBt) for 7 min at 75° C. Except Asp, Cys and His residues which were N^{α}-Fmoc deprotections at room temperature; i.e. 40% piperidine/DMF (0.2 M HOBt) for 3 min followed by 20% piperidine/DMF (0.1 M HOBt) for 15 min. The coupling chemistry was DIC (N,N′-diisopropyl-carbodiimide)/Oxyma (ethyl cyano(hydroxyimino)acetate) in DMF using amino acid solutions of 0.5 M in DMF and a molar excess of 5-fold. Standard Fmoc protected amino acids were used. Coupling conditions were single or double couplings for 15 min at 75° C. Except His and Cys residues, which were double coupled for 15 min at 50° C. Also, amino acids coupled after Aib were double coupled. The Fmoc-amino acids were dissolved at 0.5 M in DMF containing 0.5 M Oxyma, except His which was dissolved in NMP. The resin was washed 5× with DMF after N^{α}-Fmoc deprotection and 3× after couplings.

For N-terminal lipidated examples, the lipidation was conducted on-resin as the last step in the peptide synthesis. The N-terminal lipidated peptides optionally contained linker residue(s), such as [^{γ}E], [OEG], [OEG]-[OEG] etc. The linker residues are introduced by coupling Fmoc-OEG-OH, Fmoc-OEG-OEG and/or Fmoc-Glu-OtBu to the N-terminal amino acid elongating the peptide prior to coupling the fatty diacid, such as tert-butyl protected fatty diacids, such as tBu-C18-diacid, tBu-C20-diacid etc. The linker residues were double- or triple coupled using standard conditions. The fatty acid was double coupled using 2 eq. building block.

For other lipidated examples, Boc-protected amino acid was incorporated as the N-terminal residue, and the lipidation position was incorporated as orthogonal protected Lysine, here Lys(Mtt). The Mtt group (4-methyl-trityl) was removed by treating the resin with 75% HFIP (1,1,1,3,3,3-hexafluoro-propan-2-ol) in DCM (dichloromethane) plus 5% TIPS for 10 minutes. Procedure repeated 3 times. The resin was washed with 10% DIPEA in DCM, followed by 3×DMF wash.

The lipidated peptides optionally contain linker residue(s). The linker residue(s) were coupled to the epsilon-amino group of the deprotected lysine prior to coupling the fatty acid, such as tert-butyl protected fatty diacids, such as tBu-C18-diacid, tBu-C20-diacid etc. The linker residues were double- or triple coupled using standard conditions. The fatty acid was double coupled using 2 eq. building block.

After synthesis, the resin was washed with DCM and dried, and the polypeptide was cleaved from the resin by a 45 min treatment with TFA (trifluoroacetic acid)/TES (triethylsilane)/DODT/water (93/2.5/2.5/2.0) at 40° C., followed by precipitation with 3 volumes of cold diethyl ether, further washing with diethyl ether and left to dry. The peptides were characterized by LC-MS (Waters, Denmark) and quantified by LC-CAD (ThermoFisher scientific, Denmark). Finally, the peptides were freeze-dried to give a white powder using a Telstar benchtop freeze drier. Crude library peptides were used for hGLP1R and hSCTR potency screening.

### General Purification of Peptides

Crude peptide was dissolved in acetonitrile/water and purified by reverse phase HPLC using a Waters preparative HPLC with C18 column (Reprosil Gold 200 Å, 5 μm, 30 mm×250 mm), preparative pumps (waters 2545), UV/VIS detector (Waters 2489) and a Waters fraction collector III. The mobile phase was run with a gradient of buffer A (0.1% TFA in H2O) and buffer B (0.1% TFA in acetonitrile at a flow rate of 20 mL/min at room temperature. Relevant fractions were analyzed, pooled, and lyophilized. Finally, the peptide was freeze dried using a Telstar benchtop freeze drier. Peptide purity and mass were determined by analytical RP-HPLC-MS on a ACQUITY UPLC Peptide CSH C18 column (Waters, ACQUITY UPLC Peptide CSH, C18, 130 Å, 1.7 μm, 2.1 mm×100 mm) using a Waters Acquity HPLC System equipped with 3100 Mass Detector. Analysis was performed by gradient elution with buffer A (0.3% TFA in H2O) and buffer B (0.3% TFA in acetonitrile) at a temperature of 40° C. (gradients used 40-60% B over 14 min).

### General Handling of Peptides for In Vitro Potency Testing

Crude library peptides are screened in 10-fold serial dilutions of 5 concentrations, n=1 time. Tip change is done for the first two serial dilutions. Compound starting concentration (top) will be adjusted throughout the study if needed.

Purified peptides are screened in 3-fold serial dilutions of 11 concentrations, n=2 times. Tip change is done for all serial dilutions. Compound starting concentration (top) will be adjusted throughout the study. If the top concentration is adjusted or a 5-fold difference in calculated EC₅₀ values is observed, n is increased by 1. EC₅₀ values were calculated by nonlinear regression using sigmoid concentration-response with variable slope. As can be seen in table 6, potency data obtained for purified peptides correlated well with the potency data obtained for the crude peptides.

General Procedure for Determination of hGLP1R Potency

A CHO-K1 cell line stably overexpressing the hGLP1R was obtained from Euroscreen (FAST-0145L), expanded, aliquoted and frozen. An aliquot was thawed and plated in DPBS with 0.05% casein and 0.5 mM IBMX as 2000 cells/well in a 384-well format. The cells were then immediately stimulated for 30 min at room temperature with graded doses of test compound using human GLP-1(7-36) (synthesized at Gubra) as a positive control. cAMP accumulation was measured using a Cisbio assay for Gs coupled receptors (cat. no. 62AM4PEC), where the assay reagents were added as per the manufacturer's instructions and time-resolved fluorescence energy transfer recorded after one hour on a CLARIOstar (BMG Labtech) plate reader.

General Procedure for Determination of hSCTR Potency

Frozen division arrested CHO-K1 cells stably overexpressing the hSCTR were obtained from PerkinElmer (ES-712-AF). Alternatively, a CHO-K1 cell line stably overexpressing the hSCTR was obtained from Euroscreen (FAST-0161L), expanded, aliquoted and frozen. An aliquot was thawed and plated in DPBS with 0.05% casein and 0.5 mM IBMX as 2000 cells/well in a 384-well format. The cells were then immediately stimulated for 30 min at room temperature with graded doses of test compound using human secretin (synthesized at Gubra) as a positive control. cAMP accumulation was measured using a Cisbio assay for Gs coupled receptors (cat. no. 62AM4PEC), where the assay reagents were added as per the manufacturer's instructions and time-resolved fluorescence energy transfer recorded after one hour on a CLARIOstar (BMG Labtech) plate reader.

### General Procedure for Determination of Turbidity and Fibril Formation of Crude Library Peptides

Peptides were dissolved in buffers (50 mM sodium acetate at pH 4.0, or 50 mM sodium phosphate at pH 7.0) to 267 μM and incubated for 1-2 hours at room temperature. The samples were then divided into two replicates of 80 μl in a black 384 well plate (μ-clear, Greiner Bio-One) and mixed with Thioflavin T (ThT) to a final concentration of 4 μM. The plate was centrifuged for 2 min at 2000 rpm to remove air bubbles, sealed, and placed in a plate reader (CLARIOstar, BMG). Firstly, turbidity of the samples was measured as the absorbance at 600 nm. Secondly, the plate reader temperature was set to 40° C. and the fluorescence was measured every 10 min for 72 hours by exciting the ThT at 450 nm and measuring the emission at 480 nm. Samples were stressed by shaking the plate at 700 rpm (linear) for five minutes before every measurement, and fibril formation was determined as the average emission for each sample.

General Procedure for determination of fibril formation of Purified Peptides

Thioflavin T fibril formation assay: Peptides are dissolved as 267 μM in 50 mM phosphate buffer pH 7.0 for 2 h at room temperature on a rocking table. The samples were then divided into three replicates of 80 μl/well and mixed with 2 μl/well Thioflavin T (ThT) to a final concentration of 4 μM ThT in a black 384 well microplate with a clear bottom (Greiner #781096). The plate is inserted in a CLARIOstar Plus microplate reader (BMG Labtech) and fibril formation is measured as an increase in fluorescence emission at 480 nm (excitation at 450 nm) during 96 h at 40° C. with cycles of 5 min rest and 5 min linear shaking at 700 rpm.

General procedure for determination of solubility of purified Peptides.

Solubility was tested in the following vehicles: 100 mM phosphate buffer facilitating a target pH of 7.0. Samples were directly dissolved in Mini-UniPrep Syringeless Filter 0.45 μm unit (Whatman). For the desired concentration, such as a concentration of approximately 10 mg/mL, 1050 nmol of peptide was dissolved in 400 μL vehicle. Samples were incubated for at least 1 h at room temperature on a rocking table. The pH was measured and adjusted. Samples were left on the rocking table for an additional hour before a second pH measurement and adjustment. Visual inspection was performed and noted before the filter was pushed. The concentration of peptide in the filtrate was determined using CAD (Charged Aerosol Detection), double determination was done for each sample. The measured concentration was reported in μM. Furthermore, the pH of the filtrate was measured and reported. Peptides with a measured concentration within +/−20% of target concentration are considered fully soluble.

### Example 1: Identification of Important Amino Acid Residues for Improving hGLP1R and Abolishing hSCTR Potency in SCT by Introduction of GLP-1 Amino Acid Residues

Based on the native sequence of SCT, at peptide library was designed and synthesized, wherein each amino acid residue in SCT that differed from GLP1 when aligned as shown below (i.e. position X²-X³, X⁹-X¹⁰, X¹²-X¹⁴, and X¹⁷-X²⁵) was changed into the corresponding GLP-1 residue (one at a time or in combination).

| SCT | H | S | D | G | T | F | T | S | E | L | S | R | L | R | E |
|---|---|---|---|---|---|---|---|---|---|---|---|---|---|---|---|
| GLP-1 | H | A | E | G | T | F | T | S | D | V | S | S | Y | L | E |
| SCT | G | A | R | L | Q | R | L | L | Q | G | L | V | — | — | — |
| GLP-1 | G | Q | A | A | K | E | F | I | A | W | L | V | K | G | R |

This provided a library of 735 peptides, 692 peptides having a SCT backbone with 1-6 GLP-1 substitutions and 43 peptides having a SCT backbone with 14-16 GLP-1 substitutions. The EC₅₀ values on hGLP1R and hSCTR were determined and SHAP values calculated from a random forest model, where EC₅₀ values are fitted to the peptide amino acid sequences. SHAP values were used to determine the level of contribution of each amino acid substitution on the endpoints hGLP1R and hSCTR potency (Breiman, L. (2001), Random Forests, Machine Learning 45(1), 5-32; Lundberg, S. M., & Lee, S. I. (2017). A unified approach to interpreting model predictions. Advances in neural information processing systems, 30.). The contribution of each substitution was determined as the difference in average SHAP value between the SCT and GLP-1 amino acids. Substitutions with positive SHAP values increased the endpoint, while negative SHAP values decreased the endpoint.

FIGS. 1A and B show the SHAP values for the different amino acid positions in relation to hGLP1R potency and hSCTR potency. As can be seen from FIG. 1A, positions X², X⁹, X¹⁸ and X²² were found to be of the highest importance for improving the hGLP1R potency, as these have the highest positive SHAP values (cut of defined at 0.1). Likewise, as can be seen om FIG. 1B, positions X³, X⁹, X¹⁰, X¹⁴ and X¹⁹ were found to be of the highest importance for abolishing the hSCTR potency (i.e. providing selectivity), as these have the lowest negative SHAP values (cut of defined at 0.1).

### Example 2: Identification of Non-GLP-1 Residues Capable of Improving hGLP1R Potency and Abolishing hSCTR Potency in SCT

A peptide library of GLP1R agonists based on the SCT backbone was designed and synthesized, wherein each amino acid residue in SCT (i.e. positions X¹-X²⁷) was changed into all of the remaining natural amino acids or Aib (one at a time or in combination), excluding the amino acid present in SCT and GLP-1 in the respective position (also referred to as a deep mutational scan herein). This provided a library of 854 peptides each peptide having a SCT backbone with 1-4 substitutions. The inventors constructed a random forest model (Breiman, L. (2001), Random Forests, Machine Learning 45(1), 5-32.) describing the relationship between peptides sequence and hGLP1R and hSCTR potency. Besides the positions identified in example 1 using GLP-1 amino acids (i.e. positions X², X⁹, X¹⁸ for increasing hGLP1R potency and position X²² X³, X⁹, X¹⁰, X¹⁴ and X¹⁹ for abolishing hSCTR potency to provide selectivity), example 2 further identified positions X¹² and X²⁵ to further increase hGLP1R potency and abolish hSCTR potency. The random forests model was used to compute the normalized effect of changing an amino acid based on large number of peptides from the library. The data is summarized in FIGS. 2-8 for each of the positions identified.

### Example 3: Identification of Lipidation Sites without Compromising hGLP1R Potency

Optimal lipidation sites was identified by introducing lipidated lysine residues in each of the positions of native GLP-1. As shown in FIG. 12, lipidation of lysine in positions 14 and 25 had minimum adverse effect on hGLP1R potency. Thus, in a preferred embodiment of the invention, X¹⁴ or X²⁵ is selected as lysine (K), wherein the lysine is lipidated in order to alter the PK properties of the peptides.

### Example 4: Identification of Sites Suitable for Solubility Increasement without Compromising hGLP1R Potency

Positions tolerating glutamate (E) residues (i.e. without compromising hGLP1R potency) were identified in order to improve the solubility of the peptides. A library of GLP1R agonists based on the SCT backbone was constructed using the amino acids identified in examples 1 and 2. In addition, the peptides in the library had from 1 to 2 glutamate (E) substitutions in the positions X⁹-X²⁷. The hGLP1R potency was determined. The peptides in the library were lipidated in position 10, 14, 17 or 25. As can be seen from FIG. 13A, positions X¹², X⁶, X¹⁷, X²⁰, X²¹, X²⁴, X²⁵, and X²⁷ were identified as the optimal with little adverse effect or even a slightly improved effect on hGLP1R potency. In particular, X¹⁶ and X²⁴ were identified as optimal for introducing glutamate residues, most preferably position X¹⁶.

### Example 5: Identification of Sites for Improving Physical Stability

Mutations improving the physical stability (i.e. preventing fibrillation) of the peptides without compromising the hGLP1R potency were identified. A library of GLP1R agonists based on the SCT backbone was constructed using the amino acids identified in examples 1 and 2. In addition, the peptides in the library had from 1-3 mutations in the positions X³, X¹⁰, X¹², X¹⁸, X¹⁹ or X²¹. The peptides in the library were lipidated in position X¹⁴. The data are summarized in FIGS. 14A and 14B.

### Example 6: Exemplified GLP1R Agonists According to the Invention Having 27 Amino Acid Residues

Based on the key findings in examples 1-5, libraries were designed, synthesized, and tested. Table 1 shows the synthesized and tested peptides. As can be seen, the peptides incorporating the amino acids in examples 1 and 2 are highly potent and selective GLP1R agonists.

### Example 7: Exemplified Extended GLP1R Agonists Having Up to 30 Amino Acid Residues

As native GLP-1 has a length of 30 amino acids, whereas native SCT has a length of 27 amino acids, it follows that the GLP1R agonists based on the SCT backbone may be extended to the full length of native GLP-1. In order to illustrate this, a library of 28-30 amino acid long GLP1R agonists based on the SCT backbone, and mutations identified in examples 1 and 2 was constructed. The peptides were either 27, 28, 29 or 30 amino acids long. X²⁸, X²⁹ and X³⁰ were tested as either Aib, A, D, E, F, G, H, I, K, L, N, P, Q, R, S, T, V, W, or Y. Peptides in the library were not lipidated. The hGLP1R potency was determined. As shown in FIG. 15A, the tested amino acids in position X²⁸ had little effect on hGLP1R potency apart from F and P, which resulted in an adverse effect on hGLP1R potency. Furthermore, FIG. 15 shows that the amino acids A, Aib, and G in position X²⁹ had a slightly positive effect on hGLP1R potency, that amino acids D, E, L, and W had a slightly adverse effect on hGLP1R potency, whereas the remaining tested amino acids had little influence on hGLP1R potency. Finally, FIG. 15A shows that all the tested amino acids in position X³⁰ were tolerated but that the amino acid G had a slightly positive effect on hGLP1R potency. Table 2 illustrates GLP1R agonists according to the invention extended to the full length of native GLP-1.

### Example 8: Effect on Food Intake

Male NMRI mice were obtained from JanVier (JanVier Labs, France) at 5 weeks of age. The animals were group-housed 4 mice pr. cage under a 12/12 h dark-light cycle, light off at 1 PM. Room temperature was controlled to 22° C.±1° C., with 60%±20% humidity. Animals had ad libitum access to regular rodent chow (Altromin 1324, Brogaarden, Denmark) and tap water.

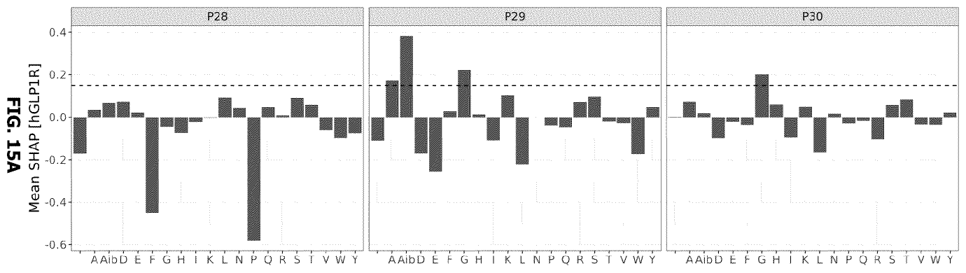

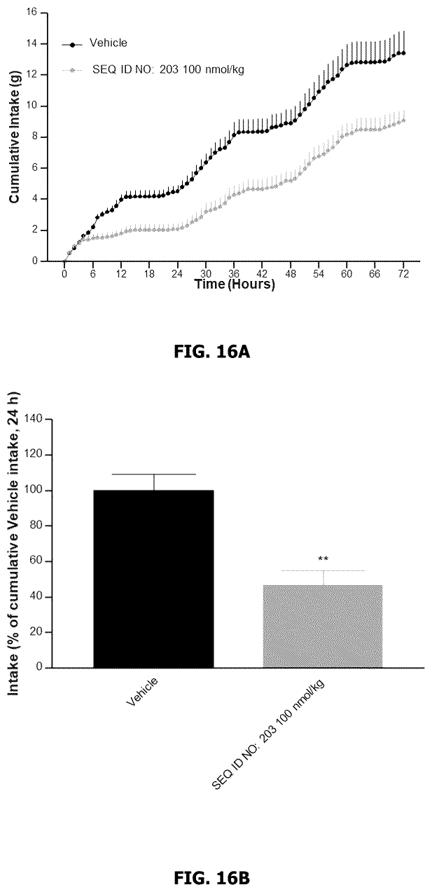

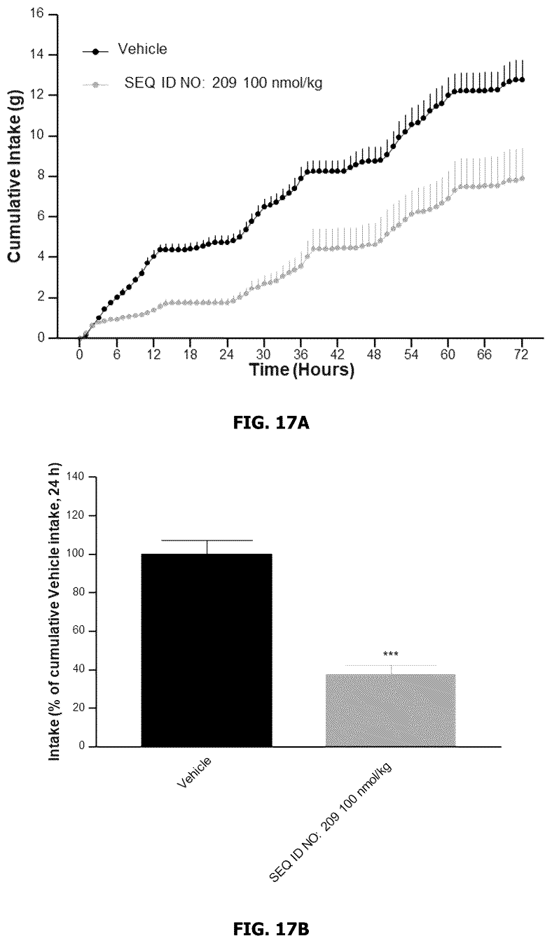

Animals were transferred 5-7 days before the start of the study to a real-time food intake monitoring system, HM-2 system (MBRose, Denmark) to allow acclimatization to experimental conditions. As the animals were uniquely identified with microchips, each individual animal was identified by its microchip upon entry and exit from the food channel. Randomization of the mice for each study group (n=7-8) was based on body weight measured the day before the start of the study. A vehicle-treated group was included in each experiment. Six hours before the start of the dark phase, animals were fasted. One hour before the dark phase, animals were dosed once subcutaneously with test peptide (see FIG. 16A/B and 17 Å/B). Formulation: 20 nmol/mL of peptide in 50 mM phosphate pH 7.4, 3.5% mannitol. Food intake was reported hourly for a period of 72 hours. The percentage of food intake reduction was normalized to average food intake from the vehicle group. Statistical significance was evaluated using One-way analysis of variance with Tukey's multiple comparison test. P<0.05 was considered statistically significant.

### LIST OF REFERENCES

Witteloostuijn et al. J. Pept. Sci. 2017 December, 23(12), page 845-854.
Breiman, L. (2001), Random Forests, Machine Learning 45(1), page 5-32.
Lundberg, S. M., & Lee, S. I., NIPS 2017, A Unified Approach to Interpreting Model Predictions, page 1-10.

TABLE 1
Exemplified peptides according to the invention

| SEQ | | | | | | | | | | | | | | | | |
|---|---|---|---|---|---|---|---|---|---|---|---|---|---|---|---|---|
| ID NO. | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ |
| 1 | H | Aib | E | G | T | F | T | S | D | V | S | R | L | K* | E | E |
| 2 | H | Aib | E | G | T | F | T | S | D | V | S | R | L | K* | E | F |
| 3 | H | Aib | E | G | T | F | T | S | D | V | S | R | L | K* | E | L |
| 4 | H | Aib | E | G | T | F | T | S | D | V | S | Y | L | K* | E | T |
| 5 | H | Aib | E | G | T | F | T | S | D | V | S | E | L | K* | E | Aib |
| 6 | H | Aib | E | G | T | F | T | S | D | V | S | T | L | K* | E | E |
| 7 | H | Aib | E | G | T | F | T | S | D | V | S | E | L | K* | E | Aib |
| 8 | H | Aib | E | G | T | F | T | S | D | V | S | E | L | K* | E | F |
| 9 | H | Aib | E | G | T | F | T | S | D | V | S | R | L | K* | E | L |
| 10 | H | Aib | E | G | T | F | T | S | D | V | S | R | L | K* | E | L |
| 11 | H | Aib | E | G | T | F | T | S | D | V | S | T | L | K* | E | L |
| 12 | H | Aib | E | G | T | F | T | S | D | V | S | E | L | K* | E | L |
| 13 | H | Aib | E | G | T | F | T | S | D | V | S | F | L | K* | E | Aib |
| 14 | H | Aib | E | G | T | F | T | S | D | V | S | E | L | K* | E | E |
| 15 | H | Aib | E | G | T | F | T | S | D | V | S | V | L | K* | E | Aib |
| 16 | H | Aib | E | G | T | F | T | S | D | V | S | T | L | K* | E | Aib |
| 17 | H | Aib | E | G | T | F | T | S | D | V | S | R | L | K* | E | E |
| 18 | H | Aib | E | G | T | F | T | S | D | V | S | E | L | K* | E | Aib |
| 19 | H | Aib | E | G | T | F | T | S | D | V | S | R | L | K* | E | L |
| 20 | H | Aib | E | G | T | F | T | S | D | V | S | T | L | K* | E | T |
| 21 | H | Aib | E | G | T | F | T | S | D | V | S | R | L | K* | E | T |
| 22 | H | Aib | E | G | T | F | T | S | D | V | S | R | L | K* | E | E |
| 23 | H | Aib | E | G | T | F | T | S | D | V | S | R | L | K* | E | T |
| 24 | H | Aib | E | G | T | F | T | S | D | V | S | R | L | K* | E | F |
| 25 | H | Aib | E | G | T | F | T | S | D | V | S | F | L | K* | E | T |
| 26 | H | Aib | E | G | T | F | T | S | D | V | S | R | L | K* | E | T |
| 27 | H | Aib | E | G | T | F | T | S | D | V | S | F | L | K* | E | F |
| 28 | H | Aib | E | G | T | F | T | S | D | V | S | V | L | K* | E | E |
| 29 | H | Aib | E | G | T | F | T | S | D | V | S | V | L | K* | E | F |
| 30 | H | Aib | E | G | T | F | T | S | D | V | S | Y | L | K* | E | E |
| 31 | H | Aib | E | G | T | F | T | S | D | V | S | E | L | K* | E | F |
| 32 | H | Aib | E | G | T | F | T | S | D | V | S | T | L | K* | E | Aib |
| 33 | H | Aib | E | G | T | F | T | S | D | V | S | V | L | K* | E | L |
| 34 | H | Aib | E | G | T | F | T | S | D | V | S | R | L | K* | E | T |
| 35 | H | Aib | E | G | T | F | T | S | D | V | S | V | L | K* | E | E |
| 36 | H | Aib | E | G | T | F | T | S | D | V | S | E | L | K* | E | L |
| 37 | H | Aib | E | G | T | F | T | S | D | V | S | F | L | K* | E | E |
| 38 | H | Aib | E | G | T | F | T | S | D | V | S | F | L | K* | E | E |
| 39 | H | Aib | E | G | T | F | T | S | D | V | S | T | L | K* | E | E |
| 40 | H | Aib | E | G | T | F | T | S | D | V | S | V | L | K* | E | Aib |
| 41 | H | Aib | E | G | T | F | T | S | D | V | S | V | L | K* | E | T |
| 42 | H | Aib | E | G | T | F | T | S | D | V | S | T | L | K* | E | T |
| 43 | H | Aib | E | G | T | F | T | S | D | V | S | T | L | K* | E | Aib |
| 44 | H | Aib | E | G | T | F | T | S | D | V | S | F | L | K* | E | T |
| 45 | H | Aib | E | G | T | F | T | S | D | V | S | E | L | K* | E | T |
| 46 | H | Aib | E | G | T | F | T | S | D | V | S | Y | L | K* | E | F |
| 47 | H | Aib | E | G | T | F | T | S | D | V | S | Y | L | K* | E | E |
| 48 | H | Aib | E | G | T | F | T | S | D | V | S | Y | L | K* | E | Aib |
| 49 | H | Aib | E | G | T | F | T | S | D | V | S | V | L | K* | E | Aib |
| 50 | H | Aib | E | G | T | F | T | S | D | V | S | R | L | K* | E | Aib |
| 51 | H | Aib | E | G | T | F | T | S | D | V | S | E | L | K* | E | E |
| 52 | H | Aib | E | G | T | F | T | S | D | V | S | R | L | K* | E | F |
| 53 | H | Aib | E | G | T | F | T | S | D | V | S | V | L | K* | E | E |
| 54 | H | Aib | E | G | T | F | T | S | D | V | S | E | L | K* | E | T |
| 55 | H | Aib | E | G | T | F | T | S | D | V | S | E | L | K* | E | E |
| 56 | H | Aib | E | G | T | F | T | S | D | V | S | F | L | K* | E | E |
| 57 | H | Aib | E | G | T | F | T | S | D | V | S | E | L | K* | E | T |
| 58 | H | Aib | E | G | T | F | T | S | D | V | S | Y | L | K* | E | T |
| 59 | H | Aib | E | G | T | F | T | S | D | V | S | R | L | K* | E | Aib |
| 60 | H | Aib | E | G | T | F | T | S | D | V | S | R | L | K* | E | E |
| 61 | H | Aib | E | G | T | F | T | S | D | V | S | Y | L | K* | E | E |
| 62 | H | Aib | E | G | T | F | T | S | D | V | S | V | L | K* | E | T |
| 63 | H | Aib | E | G | T | F | T | S | D | V | S | F | L | K* | E | E |
| 64 | H | Aib | E | G | T | F | T | S | D | V | S | R | L | K* | E | Aib |
| 65 | H | Aib | E | G | T | F | T | S | D | V | S | R | L | K* | E | E |
| 66 | H | Aib | E | G | T | F | T | S | D | V | S | Y | L | K* | E | F |
| 67 | H | Aib | E | G | T | F | T | S | D | V | S | T | L | K* | E | L |
| 68 | H | Aib | E | G | T | F | T | S | D | V | S | R | L | K* | E | F |
| 69 | H | Aib | E | G | T | F | T | S | D | V | S | Y | L | K* | E | T |
| 70 | H | Aib | E | G | T | F | T | S | D | V | S | F | L | K* | E | T |
| 71 | H | Aib | E | G | T | F | T | S | D | V | S | R | L | K* | E | T |
| 72 | H | Aib | E | G | T | F | T | S | D | V | S | R | L | K* | E | F |
| 73 | H | Aib | E | G | T | F | T | S | D | V | S | R | L | K* | E | E |
| 74 | H | Aib | E | G | T | F | T | S | D | V | S | F | L | K* | E | Aib |
| 75 | H | Aib | E | G | T | F | T | S | D | V | S | Y | L | K* | E | E |
| 76 | H | Aib | E | G | T | F | T | S | D | V | S | Y | L | K* | E | F |
| 77 | H | Aib | E | G | T | F | T | S | D | V | S | T | L | K* | E | E |
| 78 | H | Aib | E | G | T | F | T | S | D | V | S | T | L | K* | E | L |
| 79 | H | Aib | E | G | T | F | T | S | D | V | S | T | L | K* | E | L |
| 80 | H | Aib | E | G | T | F | T | S | D | V | S | E | L | K* | E | L |
| 81 | H | Aib | E | G | T | F | T | S | D | V | S | R | L | K* | E | Aib |
| 82 | H | Aib | E | G | T | F | T | S | D | V | S | E | L | K* | E | Aib |
| 83 | H | Aib | E | G | T | F | T | S | D | V | S | F | L | K* | E | E |
| 84 | H | Aib | E | G | T | F | T | S | D | V | S | F | L | K* | E | F |
| 85 | H | Aib | E | G | T | F | T | S | D | V | S | R | L | K* | E | T |
| 86 | H | Aib | E | G | T | F | T | S | D | V | S | Y | L | K* | E | L |
| 87 | H | Aib | E | G | T | F | T | S | D | V | S | E | L | K* | E | E |
| 88 | H | Aib | E | G | T | F | T | S | D | V | S | T | L | K* | E | Aib |
| 89 | H | Aib | E | G | T | F | T | S | D | V | S | T | L | K* | E | F |
| 90 | H | Aib | E | G | T | F | T | S | D | V | S | V | L | K* | E | E |
| 91 | H | Aib | E | G | T | F | T | S | D | V | S | F | L | K* | E | F |
| 92 | H | Aib | E | G | T | F | T | S | D | V | S | V | L | K* | E | F |
| 93 | H | Aib | E | G | T | F | T | S | D | V | S | T | L | K* | E | T |
| 94 | H | Aib | E | G | T | F | T | S | D | V | S | Y | L | K* | E | Aib |
| 95 | H | Aib | E | G | T | F | T | S | D | V | S | Y | L | K* | E | E |
| 96 | H | Aib | E | G | T | F | T | S | D | V | S | T | L | K* | E | E |
| 97 | H | Aib | E | G | T | F | T | S | D | V | S | E | L | K* | E | E |
| 98 | H | Aib | E | G | T | F | T | S | D | V | S | E | L | K* | E | E |
| 99 | H | Aib | E | G | T | F | T | S | D | V | S | E | L | K* | E | L |
| 100 | H | Aib | E | G | T | F | T | S | D | V | S | T | L | K* | E | F |
| 101 | H | Aib | E | G | T | F | T | S | D | V | S | V | L | K* | E | T |
| 102 | H | Aib | E | G | T | F | T | S | D | V | S | T | L | K* | E | E |
| 103 | H | Aib | E | G | T | F | T | S | D | Aib | S | Y | L | K* | E | E |
| 104 | H | Aib | E | G | T | F | T | S | D | Aib | S | R | L | L | E | G |
| 105 | H | Aib | E | G | T | F | T | S | D | Aib | S | G | L | L | E | G |
| 106 | H | Aib | E | G | T | F | T | S | D | Aib | S | Y | L | L | E | G |
| 107 | H | Aib | E | G | T | F | T | S | D | Aib | S | Aib | L | L | E | G |
| 108 | H | Aib | E | G | T | F | T | S | D | Aib | S | W | L | L | E | G |
| 109 | H | Aib | E | G | T | F | T | S | D | Aib | S | E | L | L | E | G |
| 110 | H | Aib | E | G | T | F | T | S | D | Aib | S | V | L | L | E | G |
| 111 | H | Aib | E | G | T | F | T | S | D | Aib | S | F | L | L | E | G |
| 112 | H | Aib | E | G | T | F | T | S | D | I | S | Y | L | L | E | G |
| 113 | H | Aib | E | G | T | F | T | S | D | I | S | Aib | L | L | E | G |
| 114 | H | Aib | E | G | T | F | T | S | D | I | S | Y | L | K* | E | E |
| 115 | H | Aib | E | G | T | F | T | S | D | I | S | R | L | L | E | G |
| 116 | H | Aib | E | G | T | F | T | S | D | I | S | V | L | L | E | G |
| 117 | H | Aib | E | G | T | F | T | S | D | I | S | F | L | L | E | G |
| 118 | H | Aib | E | G | T | F | T | S | D | I | S | W | L | L | E | G |
| 119 | H | Aib | E | G | T | F | T | S | D | I | S | E | L | L | E | G |
| 120 | H | Aib | E | G | T | F | T | S | D | I | S | G | L | L | E | G |
| 121 | H | Aib | E | G | T | F | T | S | D | P | S | Y | L | K* | E | E |
| 122 | H | Aib | E | G | T | F | T | S | D | P | S | E | L | L | E | G |
| 123 | H | Aib | E | G | T | F | T | S | D | P | S | G | L | L | E | G |
| 124 | H | Aib | E | G | T | F | T | S | D | P | S | R | L | L | E | G |
| 125 | H | Aib | E | G | T | F | T | S | D | P | S | Aib | L | L | E | G |
| 126 | H | Aib | E | G | T | F | T | S | D | P | S | F | L | L | E | G |
| 127 | H | Aib | E | G | T | F | T | S | D | P | S | W | L | L | E | G |
| 128 | H | Aib | E | G | T | F | T | S | D | P | S | Y | L | L | E | G |
| 129 | H | Aib | E | G | T | F | T | S | D | P | S | V | L | L | E | G |
| 130 | H | Aib | E | G | T | F | T | S | D | V | S | Y | L | W | E | E |
| 131 | H | Aib | E | G | T | F | T | S | D | V | S | Y | L | K* | E | E |
| 132 | H | Aib | E | G | T | F | T | S | D | V | S | Y | L | K* | E | E |
| 133 | H | Aib | E | G | T | F | T | S | D | V | S | Y | L | K* | E | E |
| 134 | H | Aib | E | G | T | F | T | S | D | V | S | Y | L | K* | E | E |
| 135 | H | Aib | E | G | T | F | T | S | D | V | S | Y | L | K* | E | E |
| 136 | H | Aib | E | G | T | F | T | S | D | V | S | N | L | K* | E | E |
| 137 | H | Aib | E | G | T | F | T | S | D | V | S | Y | L | K* | E | E |
| 138 | H | Aib | E | G | T | F | T | S | D | V | S | V | L | K* | E | E |
| 139 | H | Aib | E | G | T | F | T | S | D | V | S | Y | L | K* | E | E |
| 140 | H | Aib | E | G | T | F | T | S | D | V | S | P | L | K* | E | E |
| 141 | H | Aib | E | G | T | F | T | S | D | V | S | Y | L | K* | E | E |
| 142 | H | G | E | G | T | F | T | S | D | V | S | Y | L | K* | E | E |
| 143 | H | Aib | E | G | T | F | T | S | D | V | S | Y | L | Y | E | E |
| 144 | H | Aib | E | G | T | F | T | S | D | V | S | Y | L | K* | E | E |
| 145 | H | Aib | E | G | T | F | T | S | D | V | S | H | L | K* | E | E |
| 146 | H | Aib | E | G | T | F | T | S | D | V | S | Y | L | K* | E | E |
| 147 | H | Aib | E | G | T | F | T | S | D | V | S | Y | L | K* | E | E |
| 148 | H | Aib | E | G | T | F | T | S | D | V | S | A | L | K* | E | E |
| 149 | H | Aib | E | G | T | F | T | S | D | V | S | L | L | K* | E | E |
| 150 | H | Aib | E | G | T | F | T | S | D | V | S | Y | L | K* | E | E |
| 151 | H | Aib | E | G | T | F | T | S | D | V | S | Y | L | K* | E | E |
| 152 | H | Aib | E | G | T | F | T | S | D | V | S | Y | L | K* | E | E |
| 153 | H | Aib | E | G | T | F | T | S | D | V | S | D | L | K* | E | E |
| 154 | H | Aib | E | G | T | F | T | S | D | V | S | Y | L | K* | E | E |
| 155 | H | Aib | V | G | T | F | T | S | D | V | S | Y | L | K* | E | E |
| 156 | H | Aib | 0 | G | T | F | T | S | D | V | S | Y | L | K* | E | E |
| 157 | H | Aib | E | G | T | F | T | S | D | V | S | Y | L | K* | E | E |
| 158 | H | Aib | E | G | T | F | T | S | D | V | S | Y | L | Aib | E | E |
| 159 | H | Aib | Y | G | T | F | T | S | D | V | S | Y | L | K* | E | E |
| 160 | H | Aib | W | G | T | F | T | S | D | V | S | Y | L | K* | E | E |
| 161 | H | Aib | E | G | T | F | T | S | D | V | S | Y | L | K* | E | E |
| 162 | H | Aib | E | G | T | F | T | S | D | V | S | Y | L | K* | E | E |
| 163 | H | Aib | E | G | T | F | T | S | D | V | S | Aib | L | K* | E | E |
| 164 | H | Aib | E | G | T | F | T | S | D | V | S | Y | L | H | E | E |
| 165 | H | Aib | E | G | T | F | T | S | D | V | S | T | L | K* | E | E |
| 166 | H | A | E | G | T | F | T | S | D | V | S | Y | L | K* | E | E |
| 167 | H | Aib | E | G | T | F | T | S | D | V | S | G | L | K* | E | E |
| 168 | H | Aib | E | G | T | F | T | S | D | V | S | E | L | K* | E | E |
| 169 | H | Aib | E | G | T | F | T | S | D | V | S | Y | L | K* | E | E |
| 170 | H | Aib | L | G | T | F | T | S | D | V | S | Y | L | K* | E | E |
| 171 | H | Aib | H | G | T | F | T | S | D | V | S | Y | L | K* | E | E |
| 172 | H | Aib | E | G | T | F | T | S | D | V | S | I | L | K* | E | E |
| 173 | H | Aib | E | G | T | F | T | S | D | V | S | F | L | K* | E | E |
| 174 | H | Aib | E | G | T | F | T | S | D | V | S | W | L | K* | E | E |
| 175 | H | Aib | I | G | T | F | T | S | D | V | S | Y | L | K* | E | E |
| 176 | H | Aib | E | G | T | F | T | S | D | V | S | Y | L | F | E | E |
| 177 | H | Aib | E | G | T | F | T | S | D | V | S | F | L | L | E | G |
| 178 | H | Aib | E | G | T | F | T | S | D | V | S | R | L | L | E | G |
| 179 | H | Aib | E | G | T | F | T | S | D | V | S | Aib | L | L | E | G |
| 180 | H | Aib | E | G | T | F | T | S | D | V | S | E | L | L | E | G |
| 181 | H | Aib | E | G | T | F | T | S | D | V | S | Y | L | L | E | G |
| 182 | H | Aib | E | G | T | F | T | S | D | V | S | G | L | L | E | G |
| 183 | H | Aib | E | G | T | F | T | S | D | V | S | W | L | L | E | G |
| 184 | H | Aib | E | G | T | F | T | S | D | V | S | V | L | L | E | G |
| 185 | H | Aib | E | G | T | F | T | S | D | W | S | Y | L | K* | E | E |
| 186 | H | Aib | E | G | T | F | T | S | D | W | S | R | L | L | E | G |
| 187 | H | Aib | E | G | T | F | T | S | D | W | S | G | L | L | E | G |
| 188 | H | Aib | E | G | T | F | T | S | D | W | S | V | L | L | E | G |
| 189 | H | Aib | E | G | T | F | T | S | D | W | S | Y | L | L | E | G |
| 190 | H | Aib | E | G | T | F | T | S | D | W | S | F | L | L | E | G |
| 191 | H | Aib | E | G | T | F | T | S | D | W | S | W | L | L | E | G |
| 192 | H | Aib | E | G | T | F | T | S | D | W | S | Aib | L | L | E | G |
| 193 | H | Aib | E | G | T | F | T | S | D | W | S | E | L | L | E | G |
| 194 | H | Aib | E | G | T | F | T | S | D | Y | S | R | L | L | E | G |
| 195 | H | Aib | E | G | T | F | T | S | D | Y | S | G | L | L | E | G |
| 196 | H | Aib | E | G | T | F | T | S | D | Y | S | Y | L | K* | E | E |
| 197 | H | Aib | E | G | T | F | T | S | D | Y | S | Aib | L | L | E | G |
| 198 | H | Aib | E | G | T | F | T | S | D | Y | S | V | L | L | E | G |
| 199 | H | Aib | E | G | T | F | T | S | D | Y | S | E | L | L | E | G |
| 200 | H | Aib | E | G | T | F | T | S | D | Y | S | W | L | L | E | G |
| 201 | H | Aib | E | G | T | F | T | S | D | Y | S | Y | L | L | E | G |
| 202 | H | Aib | E | G | T | F | T | S | D | Y | S | F | L | L | E | G |
| 203 | H | Aib | E | G | T | F | T | S | D | V | S | Y | L | K* | E | E |
| Exem- | H | A, Aib, | I, H, | G | T | F | T | S | D | P, | S | Y, F, W, | L | F, Y, | E | E, G, |
| plified | | or G | L, W, | | | | | | | Aib, | | E, G, | | W, L, | | T, F, |
| amino | | | V, Y, | | | | | | | V, I, Y, | | V, Aib, | | H, Aib | | L, Aib |

| | | | | |
|---|---|---|---|---|
| acids | E or Q | or W | I, A, L, | or K |
| | | | T, H, P, | |
| | | | D, N, R | |

| SEQ | | | | | | | | | | | | | pEC50 | pEC50 | SCT |
|---|---|---|---|---|---|---|---|---|---|---|---|---|---|---|---|
| ID NO. | X¹⁷ | X¹⁸ | X¹⁹ | X²⁰ | X²¹ | X²² | X²³ | X²⁴ | X²⁵ | X²⁶ | X²⁷ | Selectivity | hGLPR1 | hSCTR | identity |
| 1 | A | A | Aib | Q | R | F | L | E | H | L | V | 4325 | 10.4 | 6.8 | 59 |
| 2 | A | A | A | Q | R | F | L | E | H | L | V | 6431 | 10.1 | 6.3 | 59 |
| 3 | A | A | Q | Q | R | F | L | E | H | L | V | 4851 | 10.8 | 7.1 | 59 |
| 4 | A | A | T | Q | R | F | L | E | H | L | V | 13928 | 11.1 | 7.0 | 56 |
| 5 | A | A | Q | Q | R | F | L | E | H | L | V | 6907 | 10.9 | 7.1 | 56 |
| 6 | A | A | S | Q | R | F | L | E | H | L | V | 11820 | 10.5 | 6.5 | 56 |
| 7 | A | A | Aib | Q | R | F | L | E | H | L | V | 18030 | 10.2 | 6.0 | 56 |
| 8 | A | A | S | Q | R | F | L | E | H | L | V | 30095 | 10.3 | 5.8 | 56 |
| 9 | A | A | A | Q | R | F | L | E | H | L | V | 13668 | 10.2 | 6.0 | 59 |
| 10 | A | A | Aib | Q | R | F | L | E | H | L | V | 2365 | 9.7 | 6.3 | 59 |
| 11 | A | A | A | Q | R | F | L | E | H | L | V | 11347 | 10.3 | 6.2 | 56 |
| 12 | A | A | S | O | R | F | L | E | H | L | V | 33473 | 10.7 | 6.2 | 56 |
| 13 | A | A | T | Q | R | F | L | E | H | L | V | 11130 | 10.5 | 6.5 | 56 |
| 14 | A | A | Q | Q | R | F | L | E | H | L | V | 9917 | 10.7 | 6.7 | 56 |
| 15 | A | A | A | Q | R | F | L | E | H | L | V | 3961 | 10.1 | 6.5 | 56 |
| 16 | A | A | E | Q | R | F | L | E | H | L | V | 2876 | 10.4 | 6.9 | 56 |
| 17 | A | A | S | Q | R | F | L | E | H | L | V | 10637 | 10.6 | 6.6 | 59 |
| 18 | A | A | E | Q | R | F | L | E | H | L | V | 16263 | 10.5 | 6.3 | 56 |
| 19 | A | A | S | Q | R | F | L | E | H | L | V | 7342 | 10.1 | 6.2 | 59 |
| 20 | A | A | S | Q | R | F | L | E | H | L | V | 22019 | 10.7 | 6.4 | 56 |
| 21 | A | A | Q | Q | R | F | L | E | H | L | V | 17906 | 10.9 | 6.7 | 59 |
| 22 | A | A | E | Q | R | F | L | E | H | L | V | 20678 | 10.7 | 6.4 | 59 |
| 23 | A | A | A | Q | R | F | L | E | H | L | V | 8143 | 10.7 | 6.8 | 59 |
| 24 | A | A | Q | Q | R | F | L | E | H | L | V | 2998 | 10.3 | 6.8 | 59 |
| 25 | A | A | Aib | Q | R | F | L | E | H | L | V | 8268 | 9.0 | 5.1 | 56 |
| 26 | A | A | T | Q | R | F | L | E | H | L | V | 4577 | 10.9 | 7.2 | 59 |
| 27 | A | A | Aib | Q | R | F | L | E | H | L | V | 33159 | 10.0 | 5.5 | 56 |
| 28 | A | A | A | Q | R | F | L | E | H | L | V | 55501 | 10.7 | 5.9 | 56 |
| 29 | A | A | A | Q | R | F | L | E | H | L | V | 8541 | 10.0 | 6.1 | 56 |
| 30 | A | A | T | Q | R | F | L | E | H | L | V | 22387 | 10.9 | 6.6 | 56 |
| 31 | A | A | E | Q | R | F | L | E | H | L | V | 144477 | 10.4 | 5.2 | 56 |
| 32 | A | A | Aib | Q | R | F | L | E | H | L | V | 23147 | 10.6 | 6.2 | 56 |
| 33 | A | A | Q | Q | R | F | L | E | H | L | V | 5737 | 10.2 | 6.5 | 56 |
| 34 | A | A | E | Q | R | F | L | E | H | L | V | 18582 | 10.6 | 6.4 | 59 |
| 35 | A | A | Aib | Q | R | F | L | E | H | L | V | 43142 | 10.8 | 6.1 | 56 |
| 36 | A | A | E | Q | R | F | L | E | H | L | V | 11143 | 10.5 | 6.5 | 56 |
| 37 | A | A | Aib | Q | R | F | L | E | H | L | V | 158052 | 10.9 | 5.7 | 56 |
| 38 | A | A | S | Q | R | F | L | E | H | L | V | 35785 | 10.5 | 6.0 | 56 |
| 39 | A | A | Q | Q | R | F | L | E | H | L | V | 17346 | 10.8 | 6.5 | 56 |
| 40 | A | A | Q | Q | R | F | L | E | H | L | V | 21943 | 10.7 | 6.4 | 56 |
| 41 | A | A | Q | Q | R | F | L | E | H | L | V | 16199 | 11.1 | 6.9 | 56 |
| 42 | A | A | E | Q | R | F | L | E | H | L | V | 209363 | 10.8 | 5.5 | 56 |
| 43 | A | A | A | Q | R | F | L | E | H | L | V | 36686 | 11.0 | 6.5 | 56 |
| 44 | A | A | A | Q | R | F | L | E | H | L | V | 114525 | 10.5 | 5.4 | 56 |
| 45 | A | A | Aib | Q | R | F | L | E | H | L | V | 86896 | 10.7 | 5.7 | 56 |
| 46 | A | A | Aib | Q | R | F | L | E | H | L | V | 67329 | 10.3 | 5.4 | 56 |
| 47 | A | A | Q | Q | R | F | L | E | H | L | V | 17543 | 10.8 | 6.6 | 56 |
| 48 | A | A | T | Q | R | F | L | E | H | L | V | 5236 | 10.6 | 6.9 | 56 |
| 49 | A | A | T | Q | R | F | L | E | H | L | V | 5429 | 10.7 | 6.9 | 56 |
| 50 | A | A | T | Q | R | F | L | E | H | L | V | 3425 | 10.8 | 7.3 | 59 |
| 51 | A | A | E | Q | R | F | L | E | H | L | V | 63009 | 10.5 | 5.7 | 56 |
| 52 | A | A | S | Q | R | F | L | E | H | L | V | 3341 | 9.9 | 6.4 | 59 |
| 53 | A | A | T | Q | R | F | L | E | H | L | V | 10988 | 11.3 | 7.2 | 56 |
| 54 | A | A | T | Q | R | F | L | E | H | L | V | 11020 | 10.9 | 6.9 | 56 |
| 55 | A | A | T | Q | R | F | L | E | H | L | V | 16315 | 11.0 | 6.8 | 56 |
| 56 | A | A | E | Q | R | F | L | E | H | L | V | 179887 | 10.4 | 5.1 | 56 |
| 57 | A | A | A | Q | R | F | L | E | H | L | V | 212618 | 10.6 | 5.3 | 56 |
| 58 | A | A | Q | Q | R | F | L | E | H | L | V | 35408 | 10.6 | 6.0 | 56 |
| 59 | A | A | S | Q | R | F | L | E | H | L | V | 4314 | 10.5 | 6.9 | 59 |
| 60 | A | A | A | Q | R | F | L | E | H | L | V | 9419 | 10.5 | 6.6 | 59 |
| 61 | A | A | A | Q | R | F | L | E | H | L | V | 66252 | 10.9 | 6.1 | 56 |
| 62 | A | A | A | Q | R | F | L | E | H | L | V | 18608 | 10.0 | 5.7 | 56 |
| 63 | A | A | A | Q | R | F | L | E | H | L | V | 201002 | 10.3 | 5.0 | 56 |
| 64 | A | A | Aib | Q | R | F | L | E | H | L | V | 4808 | 10.2 | 6.5 | 59 |
| 65 | A | A | T | Q | R | F | L | E | H | L | V | 6482 | 11.5 | 7.6 | 59 |
| 66 | A | A | S | Q | R | F | L | E | H | L | V | 81752 | 10.4 | 5.5 | 56 |
| 67 | A | A | T | Q | R | F | L | E | H | L | V | 7155 | 10.3 | 6.4 | 56 |
| 68 | A | A | Aib | Q | R | F | L | E | H | L | V | 11839 | 10.0 | 5.9 | 59 |
| 69 | A | A | E | Q | R | F | L | E | H | L | V | 1030149 | 10.8 | 4.8 | 56 |
| 70 | A | A | T | Q | R | F | L | E | H | L | V | 4095 | 10.7 | 7.0 | 56 |
| 71 | A | A | S | Q | R | F | L | E | H | L | V | 13611 | 10.2 | 6.0 | 59 |
| 72 | A | A | E | Q | R | F | L | E | H | L | V | 9251 | 10.0 | 6.0 | 59 |
| 73 | A | A | Q | Q | R | F | L | E | H | L | V | 10668 | 11.2 | 7.1 | 59 |
| 74 | A | A | S | Q | R | F | L | E | H | L | V | 231739 | 10.2 | 4.9 | 56 |
| 75 | A | A | S | Q | R | F | L | E | H | L | V | 133506 | 10.6 | 5.5 | 56 |
| 76 | A | A | Q | Q | R | F | L | E | H | L | V | 122180 | 10.1 | 5.0 | 56 |
| 77 | A | A | Aib | Q | R | F | L | E | H | L | V | 74783 | 10.7 | 5.8 | 56 |
| 78 | A | A | E | Q | R | F | L | E | H | L | V | 72127 | 10.3 | 5.4 | 56 |
| 79 | A | A | Aib | Q | R | F | L | E | H | L | V | 30234 | 10.0 | 5.5 | 56 |
| 80 | A | A | T | Q | R | F | L | E | H | L | V | 33512 | 10.7 | 6.1 | 56 |
| 81 | A | A | A | Q | R | F | L | E | H | L | V | 1217 | 10.1 | 7.1 | 59 |
| 82 | A | A | T | Q | R | F | L | E | H | L | V | 11264 | 11.0 | 7.0 | 56 |
| 83 | A | A | Q | Q | R | F | L | E | H | L | V | 54828 | 10.5 | 5.8 | 56 |
| 84 | A | A | A | Q | R | F | L | E | H | L | V | 51215 | 10.1 | 5.4 | 56 |
| 85 | A | A | Aib | Q | R | F | L | E | H | L | V | 12682 | 9.9 | 5.8 | 59 |
| 86 | A | A | A | Q | R | F | L | E | H | L | V | 19815 | 9.7 | 5.4 | 56 |
| 87 | A | A | S | Q | R | F | L | E | H | L | V | 39811 | 10.4 | 5.8 | 56 |
| 88 | A | A | Q | Q | R | F | L | E | H | L | V | 42658 | 11.4 | 6.8 | 56 |
| 89 | A | A | E | Q | R | F | L | E | H | L | V | 81527 | 10.7 | 5.7 | 56 |
| 90 | A | A | Q | Q | R | F | L | E | H | L | V | 51322 | 10.5 | 5.8 | 56 |
| 91 | A | A | S | Q | R | F | L | E | H | L | V | 101438 | 9.5 | 4.5 | 56 |
| 92 | A | A | S | Q | R | F | L | E | H | L | V | 20773 | 10.1 | 5.8 | 56 |
| 93 | A | A | T | Q | R | F | L | E | H | L | V | 36771 | 11.0 | 6.5 | 56 |
| 94 | A | A | Q | Q | R | F | L | E | H | L | V | 111764 | 11.0 | 6.0 | 56 |
| 95 | A | A | E | Q | R | F | L | E | H | L | V | 840427 | 10.7 | 4.8 | 56 |
| 96 | A | A | A | Q | R | F | L | E | H | L | V | 31579 | 10.9 | 6.4 | 56 |
| 97 | A | A | Aib | Q | R | F | L | E | H | L | V | 145949 | 11.0 | 5.8 | 56 |
| 98 | A | A | A | Q | R | F | L | E | H | L | V | 48854 | 10.6 | 5.9 | 56 |
| 99 | A | A | Q | Q | R | F | L | E | H | L | V | 52372 | 10.8 | 6.1 | 56 |
| 100 | A | A | Q | Q | R | F | L | E | H | L | V | 6301 | 9.9 | 6.1 | 56 |
| 101 | A | A | Aib | Q | R | F | L | E | H | L | V | 5030 | 9.7 | 6.0 | 56 |
| 102 | A | A | T | Q | R | F | L | E | H | L | V | 8851 | 11.0 | 7.1 | 56 |
| 103 | A | Aib | L | Q | R | F | L | E | H | L | V | 1181272 | 11.1 | 5.0 | 59 |
| 104 | A | A | L | Q | R | F | I | Q | W | L | V | 2902 | 9.9 | 6.5 | 67 |
| 105 | A | A | L | Q | R | F | I | Q | W | L | V | 14710 | 10.0 | 5.8 | 63 |
| 106 | A | A | L | Q | R | F | I | Q | W | L | V | 100392 | 10.8 | 5.8 | 63 |
| 107 | A | A | L | Q | R | F | I | Q | W | L | V | 6863 | 9.8 | 6.0 | 63 |
| 108 | A | A | L | Q | R | F | I | Q | W | L | V | 14555 | 10.1 | 5.9 | 63 |
| 109 | A | A | L | Q | R | F | I | Q | W | L | V | 39765 | 10.1 | 5.5 | 63 |
| 110 | A | A | L | Q | R | F | I | Q | W | L | V | 10257 | 9.7 | 5.7 | 63 |
| 111 | A | A | L | Q | R | F | I | Q | W | L | V | 70974 | 10.5 | 5.7 | 63 |
| 112 | A | A | L | Q | R | F | I | Q | W | L | V | 164097 | 10.8 | 5.6 | 63 |
| 113 | A | A | L | Q | R | F | I | Q | W | L | V | 1590 | 10.3 | 7.1 | 63 |
| 114 | A | Aib | L | Q | R | F | L | E | H | L | V | 3608 | 11.0 | 7.4 | 59 |
| 115 | A | A | L | Q | R | F | I | Q | W | L | V | 459 | 10.4 | 7.8 | 67 |
| 116 | A | A | L | Q | R | F | I | Q | W | L | V | 1422 | 10.3 | 7.1 | 63 |
| 117 | A | A | L | Q | R | F | I | Q | W | L | V | 19436 | 11.0 | 6.7 | 63 |
| 118 | A | A | L | Q | R | F | I | Q | W | L | V | 1256 | 10.2 | 7.1 | 63 |
| 119 | A | A | L | Q | R | F | I | Q | W | L | V | 2127 | 10.6 | 7.3 | 63 |
| 120 | A | A | L | Q | R | F | I | Q | W | L | V | 3717 | 10.3 | 6.7 | 63 |
| 121 | A | Aib | L | Q | R | F | L | E | H | L | V | 17452 | 8.6 | 4.4 | 59 |
| 122 | A | A | L | Q | R | F | I | Q | W | L | V | 804 | 8.2 | 5.3 | 63 |
| 123 | A | A | L | Q | R | F | I | Q | W | L | V | 671 | 8.0 | 5.2 | 63 |
| 124 | A | A | L | Q | R | F | I | Q | W | L | V | 2086 | 8.6 | 5.3 | 67 |
| 125 | A | A | L | Q | R | F | I | Q | W | L | V | 2342 | 8.4 | 5.0 | 63 |
| 126 | A | A | L | Q | R | F | I | Q | W | L | V | 253 | 8.1 | 5.7 | 63 |
| 127 | A | A | L | Q | R | F | I | Q | W | L | V | 464 | 8.1 | 5.4 | 63 |
| 128 | A | A | L | Q | R | F | I | Q | W | L | V | 1467 | 7.8 | 4.7 | 63 |
| 129 | A | A | L | Q | R | F | I | Q | W | L | V | 1220 | 8.1 | 5.0 | 63 |
| 130 | A | Aib | L | Q | R | F | L | E | K* | L | V | 34990 | 10.5 | 6.0 | 59 |
| 131 | A | Aib | N | Q | R | F | L | E | H | L | V | 996093 | 11.3 | 5.3 | 56 |
| 132 | A | L | L | Q | R | F | L | E | H | L | V | 10415 | 10.7 | 6.7 | 59 |
| 133 | A | Aib | L | Q | R | F | L | E | F | L | V | 23332 | 10.8 | 6.4 | 59 |
| 134 | A | Aib | S | Q | R | F | L | E | H | L | V | 303145 | 11.1 | 5.6 | 56 |
| 135 | A | Aib | L | Q | R | W | L | E | H | L | V | 22241 | 11.3 | 6.9 | 59 |
| 136 | A | Aib | L | Q | R | F | L | E | H | L | V | 857 | 10.9 | 7.9 | 59 |
| 137 | A | A | L | Q | R | F | L | E | H | L | V | 9970 | 11.1 | 7.1 | 59 |
| 138 | A | Aib | L | Q | R | F | L | E | H | L | V | 2569 | 10.9 | 7.5 | 59 |
| 139 | A | Aib | I | Q | R | F | L | E | H | L | V | 21979 | 11.2 | 6.9 | 56 |
| 140 | A | Aib | L | Q | R | F | L | E | H | L | V | 14381 | 8.9 | 4.7 | 59 |
| 141 | A | Aib | L | Q | R | Y | L | E | H | L | V | 59910 | 11.4 | 6.6 | 59 |
| 142 | A | Aib | L | Q | R | F | L | E | H | L | V | 1484910 | 10.9 | 4.7 | 59 |
| 143 | A | Aib | L | Q | R | F | L | E | K* | L | V | 88736 | 11.0 | 6.1 | 59 |
| 144 | A | Aib | L | Q | R | F | L | E | Y | L | V | 16042 | 10.8 | 6.6 | 59 |
| 145 | A | Aib | L | Q | R | F | L | E | H | L | V | 8838 | 11.1 | 7.2 | 59 |
| 146 | A | Aib | A | Q | R | F | L | E | H | L | V | 789678 | 11.6 | 5.7 | 56 |
| 147 | A | Aib | L | Q | R | F | L | E | K | L | V | 12361 | 11.3 | 7.2 | 59 |
| 148 | A | Aib | L | Q | R | F | L | E | H | L | V | 4094 | 10.8 | 7.2 | 59 |
| 149 | A | Aib | L | Q | R | F | L | E | H | L | V | 2619 | 10.9 | 7.5 | 59 |
| 150 | A | Aib | E | Q | R | F | L | E | H | L | V | 693266 | 11.3 | 5.5 | 56 |
| 151 | A | Aib | L | Q | R | F | L | E | E | L | V | 5170 | 10.6 | 6.9 | 59 |
| 152 | A | Aib | Aib | Q | R | F | L | E | H | L | V | 367578 | 10.9 | 5.4 | 56 |
| 153 | A | Aib | L | Q | R | F | L | E | H | L | V | 16095 | 10.7 | 6.5 | 59 |
| 154 | A | Aib | G | Q | R | F | L | E | H | L | V | 203892 | 10.8 | 5.4 | 56 |
| 155 | A | Aib | L | Q | R | F | L | E | H | L | V | 1853532 | 10.9 | 4.6 | 59 |
| 156 | A | Aib | L | Q | R | F | L | E | H | L | V | 2941711 | 10.8 | 4.4 | 59 |
| 157 | A | Aib | Q | Q | R | F | L | E | H | L | V | 121171 | 11.6 | 6.5 | 56 |
| 158 | A | Aib | L | Q | R | F | L | E | K* | L | V | 41049 | 10.4 | 5.8 | 59 |
| 159 | A | Aib | L | Q | R | F | L | E | H | L | V | 34730 | 9.1 | 4.6 | 59 |
| 160 | A | Aib | L | Q | R | F | L | E | H | L | V | 33562 | 9.1 | 4.6 | 59 |
| 161 | A | Aib | V | Q | R | F | L | E | H | L | V | 42442 | 11.4 | 6.8 | 56 |
| 162 | A | Aib | T | Q | R | F | L | E | H | L | V | 178176 | 12.2 | 6.9 | 56 |
| 163 | A | Aib | L | Q | R | F | L | E | H | L | V | 3669 | 10.9 | 7.4 | 59 |
| 164 | A | Aib | L | Q | R | F | L | E | K* | L | V | 11362 | 10.7 | 6.6 | 59 |
| 165 | A | Aib | L | Q | R | F | L | E | H | L | V | 1693 | 11.2 | 8.0 | 59 |
| 166 | A | Aib | L | Q | R | F | L | E | H | L | V | 41476 | 11.1 | 6.5 | 59 |
| 167 | A | Aib | L | Q | R | F | L | E | H | L | V | 16446 | 11.2 | 7.0 | 59 |
| 168 | A | Aib | L | Q | R | F | L | E | H | L | V | 6853 | 11.2 | 7.3 | 59 |
| 169 | A | Aib | L | Q | R | F | L | E | W | L | V | 14974 | 10.4 | 6.3 | 59 |
| 170 | A | Aib | L | Q | R | F | L | E | H | L | V | 873474 | 10.6 | 4.6 | 59 |
| 171 | A | Aib | L | Q | R | F | L | E | H | L | V | 209459 | 9.8 | 4.5 | 59 |
| 172 | A | Aib | L | Q | R | F | L | E | H | L | V | 1279 | 10.8 | 7.7 | 59 |
| 173 | A | Aib | L | Q | R | F | L | E | H | L | V | 13696 | 10.9 | 6.8 | 59 |
| 174 | A | Aib | L | Q | R | F | L | E | H | L | V | 7022 | 10.9 | 7.0 | 59 |
| 175 | A | Aib | L | Q | R | F | L | E | H | L | V | 247885 | 10.5 | 5.1 | 59 |
| 176 | A | Aib | L | Q | R | F | L | E | K* | L | V | 89023 | 10.5 | 5.5 | 59 |
| 177 | A | A | L | Q | R | F | I | Q | W | L | V | 30839 | 10.8 | 6.3 | 63 |
| 178 | A | A | L | Q | R | F | I | Q | W | L | V | 891 | 10.2 | 7.2 | 67 |
| 179 | A | A | L | Q | R | F | I | Q | W | L | V | 5995 | 10.1 | 6.3 | 63 |
| 180 | A | A | L | Q | R | F | I | Q | W | L | V | 11144 | 10.8 | 6.7 | 63 |
| 181 | A | A | L | Q | R | F | I | Q | W | L | V | 4280 | 10.3 | 6.7 | 63 |
| 182 | A | A | L | Q | R | F | I | Q | W | L | V | 2754 | 10.3 | 6.8 | 63 |
| 183 | A | A | L | Q | R | F | I | Q | W | L | V | 3127 | 10.3 | 6.8 | 63 |
| 184 | A | A | L | Q | R | F | I | Q | W | L | V | 10252 | 10.6 | 6.6 | 63 |
| 185 | A | Aib | L | Q | R | F | L | E | H | L | V | 7444 | 11.2 | 7.3 | 59 |
| 186 | A | A | L | Q | R | F | I | Q | W | L | V | 478 | 10.9 | 8.2 | 67 |
| 187 | A | A | L | Q | R | F | I | Q | W | L | V | 1437 | 10.4 | 7.3 | 63 |
| 188 | A | A | L | Q | R | F | I | Q | W | L | V | 468 | 10.1 | 7.5 | 63 |
| 189 | A | A | L | Q | R | F | I | Q | W | L | V | 10195 | 10.8 | 6.8 | 63 |
| 190 | A | A | L | Q | R | F | I | Q | W | L | V | 1844 | 10.2 | 7.0 | 63 |
| 191 | A | A | L | Q | R | F | I | Q | W | L | V | 904 | 10.1 | 7.2 | 63 |
| 192 | A | A | L | Q | R | F | I | Q | W | L | V | 383 | 9.9 | 7.3 | 63 |
| 193 | A | A | L | Q | R | F | I | Q | W | L | V | 558 | 10.6 | 7.9 | 63 |
| 194 | A | A | L | Q | R | F | I | Q | W | L | V | 114 | 10.9 | 8.8 | 67 |
| 195 | A | A | L | Q | R | F | I | Q | W | L | V | 419 | 10.6 | 8.0 | 63 |
| 196 | A | Aib | L | Q | R | F | L | E | H | L | V | 1743 | 10.9 | 7.7 | 59 |
| 197 | A | A | L | Q | R | F | I | Q | W | L | V | 492 | 10.8 | 8.1 | 63 |
| 198 | A | A | L | Q | R | F | I | Q | W | L | V | 185 | 10.6 | 8.3 | 63 |
| 199 | A | A | L | Q | R | F | I | Q | W | L | V | 223 | 10.6 | 8.3 | 63 |
| 200 | A | A | L | Q | R | F | I | Q | W | L | V | 353 | 10.7 | 8.1 | 63 |
| 201 | A | A | L | Q | R | F | I | Q | W | L | V | 940 | 10.9 | 7.9 | 63 |
| 202 | A | A | L | Q | R | F | I | Q | W | L | V | 820 | 10.4 | 7.4 | 63 |
| 203 | A | Aib | L | Q | R | F | L | E | H | L | V | 10000 | 10.7 | 6.7 | 59 |
| Exem- | A | A, Aib, | L, N, | Q | R | F, W | L, I | E, Q | W, F, | L | V | >100 | — | — | >50 |
| plified | | or L | G, S, | | | or Y | | | Y, H, | | | | | | |
| amino | | | T, Aib, | | | | | | K or | | | | | | |
| acids | | | A, E, | | | | | | E | | | | | | |
| | | | Q, V, I | | | | | | | | | | | | |

K* denotes that the lysine residue is lipidated with C20DA[YE][OEG][OEG]-, or C18DA[YE][OEG][OEG] -.

TABLE 2
Exemplified peptides according to the invention

| SEQ | | | | | | | | | | | | | | | | | |
|---|---|---|---|---|---|---|---|---|---|---|---|---|---|---|---|---|---|
| ID | | | | | | | | | | | | | | | | | |
| NO: | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ |
| 204 | H | Aib | E | G | T | F | T | S | D | V | S | R | L | K* | E | G | A |
| 205 | H | Aib | E | G | T | F | T | S | D | V | S | R | L | K* | E | G | A |
| 206 | H | Aib | E | G | T | F | T | S | D | V | S | R | L | K* | E | G | A |
| 207 | H | Aib | E | G | T | F | T | S | D | V | S | R | L | K* | E | E | A |
| 208 | H | Aib | E | G | T | F | T | S | D | V | S | R | L | K* | E | E | A |
| 209 | H | Aib | E | G | T | F | T | S | D | V | S | Y | L | K* | E | E | A |
| 210 | H | Aib | E | G | T | F | T | S | D | V | S | E | L | K* | E | E | A |
| 211 | H | Aib | E | G | T | F | T | S | D | V | S | T | L | K* | E | E | A |
| 212 | H | Aib | E | G | T | F | T | S | D | V | S | R | L | L | E | E | A |
| 213 | H | Aib | E | G | T | F | T | S | D | V | S | Y | L | L | E | E | A |
| 214 | H | Aib | E | G | T | F | T | S | D | V | S | E | L | L | E | E | A |
| 215 | H | Aib | E | G | T | F | T | S | D | V | S | R | L | K* | E | E | A |
| SEQ | | | | | | | | | | | | | | | | | |
| ID | | | | | | | | | | | | | | | PEC50 | pEC50 | SCT |
| NO: | X¹⁸ | X¹⁹ | X²⁰ | X²¹ | X²² | X²³ | X²⁴ | X²⁵ | X²⁶ | X²⁷ | X²⁸ | X²⁹ | X³⁰ | Selectivity | hGLPR1 | hSCTR | identity |
| 204 | A | E | Q | R | F | L | Q | H | L | V | E | A | E | 285714 | 11.0 | 5.6 | 67 |
| 205 | A | E | Q | R | F | L | Q | H | L | V | E | A | A | 684932 | 11.1 | 5.3 | 67 |
| 206 | A | E | Q | R | F | L | Q | H | L | V | E | A | E | 312500 | 10.8 | 5.3 | 67 |
| 207 | A | L | Q | R | F | L | E | H | L | V | E | A | A | 555 | 11.0 | 8.2 | 63 |
| 208 | Aib | L | Q | R | F | L | E | H | L | V | E | A | A | 1043 | 10.6 | 7.6 | 63 |
| 209 | Aib | L | Q | R | F | L | E | H | L | V | E | A | A | 34167 | 10.9 | 6.4 | 59 |
| 210 | Aib | L | Q | R | F | L | E | H | L | V | E | A | A | 15789 | 11.0 | 6.8 | 59 |
| 211 | Aib | L | Q | R | F | L | E | H | L | V | E | A | A | 11286 | 11.2 | 7.1 | 59 |
| 212 | Aib | L | Q | R | F | L | E | K* | L | V | E | A | A | 2864 | 10.7 | 7.2 | 63 |
| 213 | Aib | L | Q | R | F | L | E | K* | L | V | E | A | A | 54706 | 10.8 | 6.0 | 59 |
| 214 | Aib | L | Q | R | F | L | E | K* | L | V | E | A | A | 53571 | 10.9 | 6.1 | 59 |
| 215 | Aib | A | Q | R | F | L | E | H | L | V | E | A | A | 46000 | 11.0 | 6.3 | 59 |

K* denotes that the lysine residue is lipidated with C20DA[YE][OEG][OEG]-, or C18DA[YE][OEG][OEG] -.

TABLE 3
Comparison of prior art peptide GUB06-046 with position X¹⁰ (i.e. characterizing portion of claim 1)

| SEQ | | | | | | | | | | | | | | | | |
|---|---|---|---|---|---|---|---|---|---|---|---|---|---|---|---|---|
| ID | | | | | | | | | | | | | | | | |
| NO: | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ |
| 216 | H | Aib | E | G | T | F | T | S | D | L | S | R | L | L | E | G |
| (GUB06- | | | | | | | | | | | | | | | | |
| 046) | | | | | | | | | | | | | | | | |
| 217 | H | Aib | E | G | T | F | T | S | D | Y | S | R | L | L | E | G |
| 218 | H | Aib | E | G | T | F | T | S | D | I | S | R | L | L | E | |
| 219 | H | Aib | E | G | T | F | T | S | D | W | S | R | L | L | E | G |
| 220 | H | Aib | E | G | T | F | T | S | D | V | S | R | L | L | E | |
| 221 | H | Aib | E | G | T | F | T | S | D | P | S | R | L | L | E | G |
| 222 | I | Aib | E | G | T | F | T | S | D | Aib | S | R | L | L | E | G |

| SEQ | | | | | | | | | | | | | | | |
|---|---|---|---|---|---|---|---|---|---|---|---|---|---|---|---|
| ID | | | | | | | | | | | | SCT | | pEC50 | pEC50 |
| NO: | X¹⁷ | X¹⁸ | X¹⁹ | X²⁰ | X²¹ | X²² | X²³ | X²⁴ | X²⁵ | X²⁶ | X²⁷ | identity | Selectivity | hGLPR1 | hSCTR |
| 216 | A | A | L | Q | R | F | I | Q | W | L | V | 70 | 13 | 10.4 | 9.3 |
| (GUB06- | | | | | | | | | | | | | | | |
| 046) | | | | | | | | | | | | | | | |
| 217 | A | A | L | Q | R | F | I | Q | W | L | V | 67 | 114 | 10.9 | 8.8 |
| 218 | A | A | L | Q | R | F | I | Q | W | L | V | 67 | 459 | 10.4 | 7.8 |
| 219 | A | A | L | Q | R | F | I | Q | W | L | V | 67 | 478 | 10.9 | 8.2 |
| 220 | A | A | L | Q | R | F | I | Q | W | L | V | 67 | 891 | 10.2 | 7.2 |
| 221 | A | A | L | Q | R | F | I | Q | W | L | V | 67 | 2086 | 8.6 | 5.3 |
| 222 | A | A | L | Q | R | F | I | Q | W | L | V | 67 | 2902 | 9.9 | 6.5 |

TABLE 4
Comparison of prior art peptide GUB06-046 with position X12

| SEQ | | | | | | | | | | | | | | | | |
|---|---|---|---|---|---|---|---|---|---|---|---|---|---|---|---|---|
| ID | | | | | | | | | | | | | | | | |
| NO: | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ |
| 223 | H | Aib | E | G | T | F | T | S | D | L | S | Aib | L | L | E | G |
| 224 | H | Aib | E | G | T | F | T | S | D | L | S | E | L | L | E | G |
| 225 | H | Aib | E | G | T | F | T | S | D | L | S | F | L | L | E | G |
| 226 | H | Aib | E | G | T | F | T | S | D | L | S | G | L | L | E | G |
| 216 | H | Aib | E | G | T | F | T | S | D | L | S | R | L | L | E | G |
| (GUB06- | | | | | | | | | | | | | | | | |
| 046) | | | | | | | | | | | | | | | | |
| 227 | H | Aib | E | G | T | F | T | S | D | L | S | V | L | L | E | G |
| 228 | H | Aib | E | G | T | F | T | S | D | L | S | W | L | L | E | G |
| 229 | H | Aib | E | G | T | F | T | S | D | L | S | Y | L | L | E | G |

| SEQ | | | | | | | | | | | | | | | |
|---|---|---|---|---|---|---|---|---|---|---|---|---|---|---|---|
| ID | | | | | | | | | | | | SCT | | pEC50 | pEC50 |
| NO: | X¹⁷ | X¹⁸ | X¹⁹ | X²⁰ | X²¹ | X²² | X²³ | X²⁴ | X²⁵ | X²⁶ | X²⁷ | identity | Selectivity | hGLPR1 | hSCTR |
| 223 | A | A | L | Q | R | F | I | Q | W | L | V | 67 | 78 | 10.2 | 8.3 |
| 224 | A | A | L | Q | R | F | I | Q | W | L | V | 67 | 120 | 10.9 | 8.8 |
| 225 | A | A | L | Q | R | F | I | Q | W | L | V | 67 | 328 | 10.6 | 8.1 |
| 226 | A | A | L | Q | R | F | I | Q | W | L | V | 67 | 145 | 10.5 | 8.3 |
| 216 | A | A | L | Q | R | F | I | Q | W | L | V | 70 | 13 | 10.4 | 9.3 |
| (GUB06- | | | | | | | | | | | | | | | |
| 046) | | | | | | | | | | | | | | | |
| 227 | A | A | L | Q | R | F | I | Q | W | L | V | 67 | 43 | 10.5 | 8.9 |
| 228 | A | A | L | Q | R | F | I | Q | W | L | V | 67 | 53 | 10.8 | 9.1 |
| 229 | A | A | L | Q | R | F | I | Q | W | L | V | 67 | 699 | 10.8 | 8.0 |

TABLE 5
Physical stability of peptides

| | Fibril | Turbidity | SEQ ID | Fibril | Turbidity | SEQ ID | Fibril | Turbidity |
|---|---|---|---|---|---|---|---|---|
| SEQ ID NO: | formation | (pH 7) | NO: | formation | (pH 7) | NO: | formation | (pH 7) |
| 216 (GUB06-046) | 11040 | 0.48 | 39 | 9592 | 0.08 | 78 | 8551 | 0.08 |
| 1 | 11494 | 0.08 | 40 | 12180 | 0.08 | 79 | 12190 | 0.08 |
| 2 | 89647 | 0.08 | 41 | 109124 | 0.08 | 80 | 6999 | 0.08 |
| 3 | 13795 | 0.08 | 42 | 6253 | 0.08 | 81 | 10620 | 0.08 |
| 4 | 32222 | 0.08 | 43 | 18355 | 0.09 | 82 | 8549 | 0.08 |
| 5 | 14563 | 0.09 | 44 | 10742 | 0.08 | 83 | 9070 | 0.08 |
| 6 | 6489 | 0.09 | 45 | 14290 | 0.09 | 84 | 47022 | 0.08 |
| 7 | 10801 | 0.08 | 46 | 16732 | 0.08 | 85 | 19223 | 0.09 |
| 8 | 259534 | 0.09 | 47 | 13638 | 0.08 | 86 | 9344 | 0.08 |
| 9 | 10378 | 0.08 | 48 | 16290 | 0.08 | 87 | 7811 | 0.08 |
| 10 | 6765 | 0.08 | 49 | 11371 | 0.08 | 88 | 10893 | 0.08 |
| 11 | 7854 | 0.08 | 50 | 12284 | 0.09 | 89 | 14469 | 0.08 |
| 12 | 7904 | 0.09 | 51 | 8475 | 0.09 | 90 | 5668 | 0.09 |
| 13 | 16121 | 0.08 | 52 | 241858 | 0.08 | 91 | 260000 | 0.42 |
| 14 | 10247 | 0.08 | 53 | 5164 | 0.08 | 92 | 260000 | 0.14 |
| 15 | 12385 | 0.09 | 54 | 7756 | 0.09 | 93 | 6728 | 0.08 |
| 16 | 11719 | 0.08 | 55 | 6162 | 0.09 | 94 | 10901 | 0.08 |
| 17 | 10330 | 0.08 | 56 | 11030 | 0.10 | 95 | 10160 | 0.08 |
| 18 | 12140 | 0.09 | 57 | 4882 | 0.08 | 96 | 8019 | 0.09 |
| 19 | 170210 | 0.10 | 58 | 22040 | 0.08 | 97 | 10277 | 0.09 |
| 20 | 33108 | 0.10 | 59 | 13939 | 0.09 | 98 | 10655 | 0.09 |
| 21 | 193729 | 0.08 | 60 | 11873 | 0.09 | 99 | 87225 | 0.17 |
| 22 | 16453 | 0.08 | 61 | 9138 | 0.09 | 100 | 24482 | 0.08 |
| 23 | 7462 | 0.08 | 62 | 21979 | 0.08 | 101 | 8172 | 0.08 |
| 24 | 221619 | 0.08 | 63 | 9044 | 0.09 | 102 | 6284 | 0.08 |
| 25 | 8584 | 0.08 | 64 | 14614 | 0.09 | 103 | 3646 | 0.08 |
| 26 | 97558 | 0.08 | 65 | 9674 | 0.09 | 104 | 1348 | 0.12 |
| 27 | 16757 | 0.09 | 66 | 201306 | 0.08 | 105 | 4216 | 0.12 |
| 28 | 7910 | 0.09 | 67 | 5041 | 0.08 | 106 | 11918 | 0.74 |
| 29 | 28786 | 0.08 | 68 | 13444 | 0.08 | 107 | 3284 | 0.15 |
| 30 | 6899 | 0.08 | 69 | 14799 | 0.08 | 108 | 20603 | 0.72 |
| 31 | 10417 | 0.08 | 70 | 99118 | 0.11 | 109 | 3301 | 0.08 |
| 32 | 16551 | 0.08 | 71 | 231646 | 0.08 | 110 | 3449 | 0.15 |
| 33 | 45087 | 0.08 | 72 | 96622 | 0.08 | 111 | 26712 | 0.24 |
| 34 | 11365 | 0.08 | 73 | 14344 | 0.09 | 112 | 93630 | 0.29 |
| 35 | 8261 | 0.08 | 74 | 12039 | 0.08 | 113 | 11510 | 0.78 |
| 36 | 9625 | 0.09 | 75 | 9457 | 0.08 | 114 | 4292 | 0.08 |
| 37 | 12367 | 0.08 | 76 | 20479 | 0.08 | 115 | 6433 | 0.48 |
| 38 | 38424 | 0.09 | 77 | 11936 | 0.08 | 116 | 202498 | 0.42 |
| 117 | 238242 | 0.50 | 146 | 3918 | 0.08 | 175 | 3296 | 0.08 |
| 118 | 143374 | 0.63 | 147 | 4336 | 0.08 | 176 | 3751 | 0.08 |
| 119 | 13897 | 0.12 | 148 | 2928 | 0.08 | 177 | 260000 | 0.53 |
| 120 | 4449 | 0.23 | 149 | 2878 | 0.08 | 178 | 29072 | 0.77 |
| 121 | 24767 | 0.08 | 150 | 2766 | 0.08 | 179 | 22028 | 0.55 |
| 122 | 3560 | 0.08 | 151 | 3061 | 0.08 | 180 | 53478 | 0.12 |
| 123 | 3764 | 0.11 | 152 | 3105 | 0.08 | 181 | 257400 | 0.69 |
| 124 | 1370 | 0.12 | 153 | 3897 | 0.08 | 182 | 253106 | 0.40 |
| 125 | 4675 | 0.11 | 154 | 3107 | 0.08 | 183 | 172171 | 0.67 |
| 126 | 41799 | 0.26 | 155 | 4368 | 0.08 | 184 | 260000 | 0.43 |
| 127 | 11084 | 0.19 | 156 | 3949 | 0.08 | 185 | 4198 | 0.08 |
| 128 | 6144 | 0.20 | 157 | 3415 | 0.09 | 186 | 48618 | 1.04 |
| 129 | 5327 | 0.12 | 158 | 4465 | 0.08 | 187 | 13388 | 0.62 |
| 130 | 4181 | 0.08 | 159 | 5426 | 0.08 | 188 | 260000 | 0.85 |
| 131 | 2709 | 0.09 | 160 | 4754 | 0.09 | 189 | 94029 | 0.37 |
| 132 | 3358 | 0.08 | 161 | 3973 | 0.08 | 190 | 260000 | 0.61 |
| 133 | 4303 | 0.08 | 162 | 4281 | 0.08 | 191 | 120310 | 0.61 |
| 134 | 3061 | 0.08 | 163 | 3983 | 0.08 | 192 | 29535 | 0.54 |
| 135 | 4491 | 0.09 | 164 | 4789 | 0.08 | 193 | 26948 | 0.13 |
| 136 | 3353 | 0.08 | 165 | 4038 | 0.08 | 194 | 8355 | 0.27 |
| 137 | 4019 | 0.09 | 166 | 4253 | 0.08 | 195 | 144234 | 0.25 |
| 138 | 3306 | 0.09 | 167 | 4018 | 0.08 | 196 | 5187 | 0.08 |
| 139 | 4010 | 0.09 | 168 | 2998 | 0.08 | 197 | 7498 | 0.21 |
| 140 | 2733 | 0.08 | 169 | 3552 | 0.08 | 198 | 257659 | 0.33 |
| 141 | 3233 | 0.08 | 170 | 3337 | 0.08 | 199 | 260000 | 0.46 |
| 142 | 3679 | 0.08 | 171 | 3473 | 0.08 | 200 | 141730 | 0.92 |
| 143 | 5733 | 0.08 | 172 | 2942 | 0.08 | 201 | 30208 | 0.30 |
| 144 | 5805 | 0.08 | 173 | 2836 | 0.08 | 202 | 260000 | 0.60 |
| 145 | 2891 | 0.08 | 174 | 3729 | 0.08 | — | — | — |

TABLE 6
Characterization of selected purified peptides

| | | hGLPR1 | | hSCTR | | | Solubility (pH 7) |
|---|---|---|---|---|---|---|---|
| SEQ ID NO: | Selectivity | EC50 nM | PEC50 | EC50 nM | pEC50 | Fibril formation | mg/ml |
| 168 | 8333 | 0.012 | 10.9 | 100 | 7.0 | No | 9.8 |
| 203 | 11176 | 0.017 | 10.8 | 190 | 6.7 | No | 9.8 |
| 208 | 1091 | 0.022 | 10.6 | 24 | 7.6 | No | 9.2 |
| 209 | 33673 | 0.0098 | 11.0 | 330 | 6.5 | No | 11 |
| 210 | 20000 | 0.0095 | 11.0 | 190 | 6.7 | No | 8.3 |
| 212 | 2857 | 0.021 | 10.7 | 60 | 7.2 | No | 8.4 |
| 213 | 63571 | 0.014 | 10.9 | 890 | 6.1 | No | 8.2 |

We claim:

1.-25. (canceled)

26. A polypeptide or a pharmaceutically acceptable salt thereof comprising the structure of Formula (I), 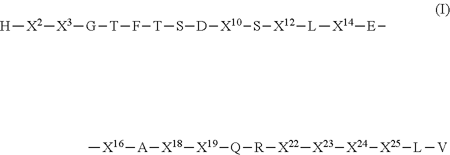
wherein,
X² is selected as A, Aib, or G;
X³ is selected as I, H, L, W, V, Y, E or Q;
X¹² is selected as Y, F, W, E, G, V, Aib, I, A, L, T, H, P, D, N, or R;
X¹⁴ is selected as F, Y, W, L, H, Aib or K
X¹⁶ is selected as E, G, T, F, L, Aib;
X¹⁸ is selected as A, Aib, or L;
X¹⁹ is selected as L, N, G, S, T, Aib, A, E, Q, I or V.
X²² is selected as F, W or Y;
X²³ is selected as L or I;
X²⁴ is selected as E or Q;
X²⁵ is selected as W, F, Y, H, K or E;
wherein X¹⁰ is selected as P, Aib, V, I, Y, or W and in that said polypeptide has a SCTR EC₅₀/GLP1R EC₅₀ ratio of at least 100.

27. The polypeptide or a pharmaceutically acceptable salt thereof according to claim 26, wherein X³ is selected as I, H, L, E, or Q.

28. The polypeptide or a pharmaceutically acceptable salt thereof according to claim 26, wherein X¹⁰ is selected as V, I, Y, or W.

29. The polypeptide or a pharmaceutically acceptable salt thereof according to claim 26, wherein X¹⁴ is selected as F, Y, W, L, H, or K, most preferably X¹⁴ is selected as L or K.

30. The polypeptide or a pharmaceutically acceptable salt thereof according to claim 26, wherein X¹⁹ is selected as L.

31. The polypeptide or a pharmaceutically acceptable salt thereof according to claim 26, wherein X¹² is selected as Y, F, W, E, G, V, or Aib, preferably X¹² is selected as Y, F, W, or E, most preferably X¹² is selected as Y.

32. The polypeptide or a pharmaceutically acceptable salt thereof according to claim 26, wherein X²³ is selected as L.

33. The polypeptide or a pharmaceutically acceptable salt thereof according to claim 26, wherein X²⁵ is selected as W, F, Y, H, K or E, most preferably X²⁵ is selected as K or H.

34. The polypeptide or a pharmaceutically acceptable salt thereof according to claim 26, wherein at least one of X¹², X¹⁶, or X²⁴ is selected as E, preferably X¹⁶ and/or X²⁴ is selected as E, most preferably X¹⁶ and X²⁴ are selected as E.

35. The polypeptide or a pharmaceutically acceptable salt thereof according to claim 26, comprising the structure of Formula (I), 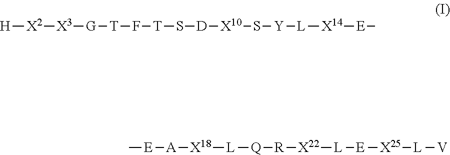
wherein,
X² is selected as A, Aib, or G;
X³ is selected as I, H, L, E, or Q;
X¹⁴ is selected as L, or K;
X¹⁸ is selected as A, Aib, or L;
X²² is selected as F, W or Y;
X²⁵ is selected as H or K;
characterized in that, X¹⁰ is selected as V, I, Y, or W.

36. The polypeptide or a pharmaceutically acceptable salt thereof according to claim 26, wherein X² is selected as Aib.

37. The polypeptide or a pharmaceutically acceptable salt thereof according to claim 26, wherein X³ is selected as E.

38. The polypeptide or a pharmaceutically acceptable salt thereof according to claim 26, wherein X¹⁰ is selected as V.

39. The polypeptide or a pharmaceutically acceptable salt thereof according to claim 26, wherein X¹⁸ is selected as Aib.

40. The polypeptide or a pharmaceutically acceptable salt thereof according to claim 26, wherein X²² is selected as F.

41. The polypeptide or a pharmaceutically acceptable salt thereof according to claim 26, wherein only one of X¹⁴ or X²⁵ is selected as K, and wherein the K is lipidated optionally through a linker/spacer.

42. The polypeptide or a pharmaceutically acceptable salt thereof according to claim 26, wherein X¹⁴ is selected as K and X²⁵ is selected as H, and wherein the K is lipidated optionally through a linker/spacer; or wherein X¹⁴ is selected as L and X²⁵ is selected as K, and wherein the K is lipidated optionally through a linker/spacer.

43. The polypeptide or a pharmaceutically acceptable salt thereof according to claim 26, wherein the polypeptide comprises 30 amino acid residues (X¹-X³⁰).

44. The polypeptide or a pharmaceutically acceptable salt thereof according to claim 26, wherein said polypeptide comprises the sequence of H[Aib]EGTFTSDVSYLLEEA[Aib]LQRFLEKLVEAA (SEQ ID NO: 213); H[Aib]EGTFTSDVSYLKEEA[Aib]LQRFLEHLVEAA (SEQ ID NO: 209); or H[Aib]EGTFTSDVSYLKEEA[Aib]LQRFLEHLV (SEQ ID NO: 203).

45. The polypeptide or a pharmaceutically acceptable salt thereof according to claim 26, wherein said polypeptide consists of the sequence of H[Aib]EGTFTSDVSYLLEEA[Aib]LQRFLE[K*]LVEAA (SEQ ID NO: 213); H[Aib]EGTFTSDVSYL[K*]EEA[Aib]LQRFLEHLVEAA (SEQ ID NO: 209); or H[Aib]EGTFTSDVSYL[K*]EEA[Aib]LQRFLEHLV (SEQ ID NO: 203), wherein * denotes a covalent attachment of a C20DA[^{γ}E][OEG][OEG]- to the lysine.

46. The polypeptide or a pharmaceutically acceptable salt thereof according to claim 26, wherein the polypeptide is amidated (—COHN) at the C-terminal.

47. The polypeptide or a pharmaceutically acceptable salt thereof according to claim 26, wherein the polypeptide has an amino group (—NH₂) at the N-terminal.

48. A polypeptide or a pharmaceutically acceptable salt thereof according to claim 26, for use as a medicament; preferably for use as a medicament for the treatment of obesity, diabetes, renal diseases, and/or non-alcoholic steatohepatitis (NASH); most preferably for use as a medicament for the treatment of obesity and/or diabetes.

49. A pharmaceutical composition comprising a polypeptide or a pharmaceutically acceptable salt thereof according to claim 26.
